(19) United States
(12) Patent Application Publication
(10) Pub. No.: US 2010/0104659 A1
(43) Pub. Date: Apr. 29, 2010

(54) BENZOPYRANOPYRAZOLES

(76) Inventors: Matthias Vennemann, Konstanz (DE); Thomas Bär, Reichenau (DE); Thomas Maier, Stockach (DE); Andreas Lindenmaier, Steinen-Holistein (DE); Jürgen Braunger, Modling (AT); Markus Boehm, Rheinfelden (DE); Astrid Zimmermann, Muhltal (DE); Volker Gekeler, Konstanz (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/373,433

(22) PCT Filed: Jul. 12, 2007
(86) PCT No.: PCT/EP2007/057195

§ 371 Date: Sep. 28, 2009

(30) Foreign Application Priority Data
Jul. 13, 2006 (EP) 06117124.5

(51) Int. Cl. A61K 31/4162; C07D 491/04; C07D 471/02; C07D 401/14; C07D 403/14; A61K 31/4738; A61K 31/454; A61K 31/496; A61K 33/24; A61P 35/00; A61K 31/397

(52) U.S. Cl. 424/649; 548/359.1; 546/82; 546/199; 544/371; 514/403; 514/293; 514/322; 514/254.06; 514/210.21

(57) ABSTRACT

Compounds of a certain formula I, in which Ra, Rb and Rc have the meanings indicated in the description, are effective compounds with anti-proliferative and/or apoptosis inducing activity.

### FIELD OF APPLICATION OF THE INVENTION

The invention relates to benzopyranopyrazole (dihydrochromenopyrazole) derivatives, which can be used in the pharmaceutical industry for the production of pharmaceutical compositions.

### KNOWN TECHNICAL BACKGROUND

The synthesis of certain tricyclic heterocyclic compounds, inter alia of benzopyranopyrazoles, and the anticancer activity of some of these compounds is described by Hammam et. al in the Indian Journal of Chemistry, (2003), 42B, 1985-93.

The synthesis and characterization of certain heterocyclic compounds, inter alia of benzopyranopyrazoles derivatives, is described by Sangwan et. al. in the Indian Journal of Chemistry, (1981), 20B, 135 which compounds are designed as potential non-steroidal antifertility agents.

The synthesis and characterization inter alia of certain benzopyranopyrazoles is described for example by Toth et. al. in J. Chem. Soc. Perkin Trans. 2, (1989), 319, by N. K. Sangwan in J. Chem. Research (S), (1987), 22-23 or by A. Levai in J. Heterocyclic Chem. (2004), 41, 299.

In the international patent application WO 90/03969 N-acylated pyrazoline arthropodicides are described.

The compound 3-phenyl-3a,4-dihydro-3H-chromeno[4,3-c]pyrazole-2-carboxylic acid amide is contained in the database of the in vitro cancer cell line screening project (IVCLSP) of the US National Cancer Institute (NCI) having the NSC-No. 652810.

In the international patent application WO2006/086358, tricyclic pyrazoles are described that are said to be useful for treating cellular proliferative diseases, for treating disorders associated with KSP kinesin activity, and for inhibiting KSP kinesin.

In the US patent application US2003/0114432, substituted pyrazolyl derivatives are described, together with methods for treating inter alia cancer.

In the US patent application US2005/004156, compounds are described that are said to be capable of inhibiting mitosis in metabolically active cells.

### DESCRIPTION OF THE INVENTION

It has now been found, that the benzopyrano[4,3-c]pyrazole (dihydrochromeno[4,3-c]pyrazole) derivatives, which are described in greater details below, differ from prior art compounds by unanticipated structural features and have surprising and particularly advantageous properties. Thus, for example, the compounds according to this invention can act as inhibitors of Eg5 kinesin. In more detail, it has been unexpectedly found that these derivatives are potent and highly efficacious inhibitors of cellular (hyper)proliferation and/or cell-cycle specific inducers of apoptosis in cancer cells. Therefore, these compounds can be particular useful for treating (hyper)proliferative diseases and/or disorders responsive to the induction of apoptosis, notably cancer. By having a cell-cycle specific mode of action, these derivatives should have a higher therapeutic index compared to standard chemotherapeutic drugs targeting basic cellular processes like DNA replication or interfering with basic cellular molecules like DNA.

Thus, for example, the compounds according to this invention are expected to be useful in targeted cancer therapy.

The invention thus relates to compounds of formula I

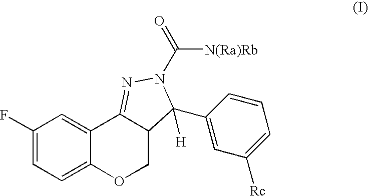

in which
Ra is 1-4C-alkyl, 3-7C-cycloalkyl, 3-7C-cycloalkyl-1-4C-alkyl, HetA, HetA-1-4C-alkyl, completely or partially fluorine-substituted 1-4C-alkyl, or 1-4C-alkyl substituted by Raa,
   wherein said 3-7C-cycloalkyl alone or as part of another group may be optionally substituted by one or two substituents independently selected from —N(R2)R3 and R1,

in which
Raa is —N(R2)R3, chlorine, bromine, hydroxyl, or 1-4C-alkoxy,
HetA is tetrahydropyranyl, tetrahydrofuranyl, 1N—(R10)-piperidinyl, or 1N—(R10)-pyrrolidinyl, in which
R10 is hydrogen, 1-4C-alkyl, 3-7C-cycloalkyl, 3-7C-cycloalkyl-1-4C-alkyl, 1-4C-alkylcarbonyl, amidino, or completely or partially fluorine-substituted 1-4C-alkyl,
wherein said HetA alone or as part of another group may be optionally substituted by one or two substituents independently selected from R1,
R1 is fluorine, or completely or partially fluorine-substituted 1-4C-alkyl,
R2 is hydrogen, 1-4C-alkyl, 3-7C-cycloalkyl, 3-7C-cycloalkyl-1-4C-alkyl, 2-4C-alkenyl, 2-4C-alkynyl, 1-4C-alkoxycarbonyl, hydroxy-2-4C-alkyl, 1-4C-alkoxy-2-4C-alkyl, HetA, HetA-1-4C-alkyl, or completely or partially fluorine-substituted 1-4C-alkyl,
R3 is hydrogen, 1-4C-alkyl, 3-7C-cycloalkyl, or 3-7C-cycloalkyl-1-4C-alkyl,
or R2 and R3 together and with inclusion of the nitrogen atom, to which they are bonded, form a ring HetB, in which
HetB is piperidin-1-yl, morpholin-4-yl, thiomorpholin-4-yl, S-oxo-thiomorpholin-4-yl, S,S-dioxo-thiomorpholin-4-yl, pyrrolidin-1-yl, azetidin-1-yl, homopiperidin-1-yl, 4N—(R21)-piperazin-1-yl, 4N—(R21)-homopiperazin-1-yl, pyrrol-1-yl, pyrazol-1-yl, imidazol-1-yl, triazol-1-yl, or tetrazol-1-yl, in which
R21 is hydrogen, 1-4C-alkyl, 3-7C-cycloalkyl, 3-7C-cycloalkyl-1-4C-alkyl, 1-4C-alkylcarbonyl, amidino, or completely or partially fluorine-substituted 1-4C-alkyl,
wherein said HetB may be optionally substituted by one or two substituents independently selected from fluorine and 1-4C-alkyl,
Rb is 1-4C-alkyl, 3-7C-cycloalkyl, or 3-7C-cycloalkyl-1-4C-alkyl,
Rc is hydrogen or hydroxyl,
and the salts, stereoisomers and the salts of the stereoisomers of these compounds.

As used herein, “alkyl” alone or as part of another group refers to both branched and straight chain saturated aliphatic hydrocarbon groups having the specified numbers of carbon atoms, such as for example:

1-4C-Alkyl is a straight-chain or branched alkyl radical having 1 to 4 carbon atoms. Examples are the butyl, isobutyl, sec-butyl, tert-butyl, propyl, isopropyl, ethyl and methyl radicals, of which propyl, isopropyl, ethyl and methyl are more worthy to be mentioned.

2-4C-Alkyl is a straight-chain or branched alkyl radical having 2 to 4 carbon atoms. Examples are the butyl, isobutyl, sec-butyl, tert-butyl, propyl, isopropyl and ethyl radicals, of which propyl, isopropyl and ethyl are more worthy to be mentioned.

1-4C-Alkoxy represents radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy, ethoxy and methoxy radicals, of which propoxy, isopropoxy, and, particularly, ethoxy and methoxy are more worthy to be mentioned.

The term “cycloalkyl” alone or as part of another group refers to a monocyclic saturated aliphatic hydrocarbon group having the specified numbers of ring carbon atoms, such as for example: 3-7C-Cycloalkyl stands for cyclopropyl, cyclobutyl, cyclopentyl, cyclohexyl and cycloheptyl.

3-7C-Cycloalkyl-1-4C-alkyl stands for one of the abovementioned 1-4C-alkyl radicals, which is substituted by one of the abovementioned 3-7C-cycloalkyl radicals. Examples which may be mentioned are the 3-7C-cycloalkylmethyl and the 2-(3-7C-cycloalkyl)ethyl radicals, particularly the cyclopropylmethyl, the cyclohexylmethyl and the 2-cyclohexylethyl radicals.

As completely or partially fluorine-substituted 1-4C-alkyl, for example, the 2,2,3,3,3-pentafluoropropyl, the perfluoroethyl, the 1,2,2-trifluoroethyl, the 1,1,2,2-tetrafluoroethyl, the 2,2,2-trifluoroethyl, the trifluoromethyl, the difluoromethyl, the monofluoromethyl, the 2-fluoroethyl and the 2,2-difluoroethyl radicals may be mentioned, particularly the 2,2,2-trifluoroethyl, 2,2-difluoroethyl and 2-fluoroethyl radicals.

1-4C-Alkylcarbonyl is a carbonyl group, to which one of the abovementioned 1-4C-alkyl radicals is bonded. An example is the acetyl radical (CH₃CO—).

2-4C-Alkenyl represents straight-chain or branched alkenyl groups having 2 to 4 carbon atoms. Examples which may be mentioned are the 2-butenyl, 3-butenyl, 1-propenyl and the 2-propenyl group (allyl group).

2-4C-Alkynyl represents straight-chain or branched alkynyl groups having 2 to 4 carbon atoms. Examples which may be mentioned are the 2-butynyl, 3-butynyl, and preferably the 2-propynyl group (propargyl group).

1-4C-Alkoxycarbonyl (—CO-1-4C-alkoxy) represents a carbonyl group, to which one of the aforementioned 1-4C-alkoxy groups is bonded. Examples which may be mentioned are the methoxycarbonyl (CH₃O—C(O)—) and the ethoxycarbonyl group (CH₃CH₂O—C(O)—). An example especially to be emphasized is the tert-butyloxycarbonyl group (Me₃C—O—C(O)—, Boc, protecting group).

HetA-1-4C-alkyl stands for one of the abovementioned 1-4C-alkyl radicals, which is substituted by an HetA radical as defined herein, whereby the HetA moiety is attached to the 1-4C-alkyl radical via a ring carbon atom, such as e.g. the HetA-methyl, 2-HetA-ethyl or 3-HetA-propyl radical.

HetA is attached to the parent molecular group via a ring carbon atom, and is optionally substituted by one or two substituents independently selected from R1, and is tetrahydropyranyl, tetrahydrofuranyl, 1N—(R10)-piperidinyl (e.g. 1N—(R10)-piperidin-3-yl or 1N—(R10)-piperidin-4-yl), or 1N—(R10)-pyrrolidinyl (e.g. 1N—(R10)-pyrrolidin-3-yl), in which
R10 is hydrogen, 1-4C-alkyl, 3-7C-cycloalkyl, 3-7C-cycloalkyl-1-4C-alkyl, 1-4C-alkylcarbonyl, amidino, or completely or partially fluorine-substituted 1-4C-alkyl, and
R1 is fluorine, or completely or partially fluorine-substituted 1-4C-alkyl;

in particular
R10 is hydrogen, 1-3C-alkyl, cyclopropyl, cyclopropylmethyl, 1-2C-alkylcarbonyl, amidino, or partially fluorine-substituted 1-3C-alkyl (e.g. 2-fluoroethyl, 2,2,2-trifluoroethyl or, particularly, 2,2-difluoroethyl), and
R1 is fluorine, or partially fluorine-substituted 1-2C-alkyl (e.g. monofluoromethyl).

In a first embodiment, HetA is optionally substituted by one substituent selected from R1, and is N—(R10)-piperidinyl, such as e.g. 1N—(R10)-piperidin-3-yl or 1N—(R10)-piperidin-4-yl, in which
R10 is hydrogen, methyl, ethyl, isopropyl, cyclopropyl, cyclopropylmethyl, 1-2C-alkylcarbonyl, amidino, 2-fluoroethyl, 2,2,2-trifluoroethyl or 2,2-difluoroethyl, and
R1 is fluorine, or fluoromethyl.

One group of HetA radicals according to this first embodiment include unsubstituted HetA radicals; such as, for example, 1NH-piperidinyl (e.g. 1H-piperidin-3-yl or 1H-piperidin-4-yl), 1N-(1-3C-alkyl)-piperidinyl (e.g. 1-methyl-piperidin-3-yl, 1-ethyl-piperidin-3-yl, 1-isopropyl-piperidin-3-yl, 1-methyl-piperidin-4-yl, 1-ethyl-piperidin-4-yl or 1-isopropyl-piperidin-4-yl), N-cyclopropyl-piperidinyl (e.g. 1-cyclopropyl-piperidin-3-yl or 1-cyclopropyl-piperidin-4-yl), 1N-(partially fluorine-substituted ethyl)-piperidinyl such as e.g. 1-(2,2,2-trifluoroethyl)-piperidinyl, 1-(2,2-difluoroethyl)-piperidinyl or 1-(2-fluoroethyl)-piperidinyl (e.g. 1-(2,2-difluoroethyl)-piperidin-3-yl or 1-(2,2-difluoroethyl)-piperidin-4-yl), 1N-(amidino)-piperidinyl (e.g. 1-amidino-piperidin-3-yl or 1-amidino-piperidin-4-yl), or 1N-(1-2C-alkylcarbonyl)-piperidinyl (e.g. 1-acetyl-piperidin-3-yl or 1-acetyl-piperidin-4-yl).

Another group of HetA radicals according to this first embodiment include HetA radicals substituted by R1, e.g. those in which R1 is fluorine; such as, for example, 1N—(R10)-fluoro-piperidinyl (e.g. 1N—(R10)-3-fluoro-piperidin-4-yl), e.g. the fluoro-piperidinyl derivatives of the aforementioned exemplary HetA radicals, such as 1N-(1-3C-alkyl)-fluoro-piperidinyl (like 1-methyl-3-fluoro-piperidin-4-yl).

In a second embodiment, HetA is optionally substituted by one substituent selected from R1, and is 1N—(R10)-pyrrolidinyl, such as e.g. 1N—(R10)-pyrrolidin-3-yl, in which
R10 is hydrogen, methyl, ethyl, isopropyl, cyclopropyl, cyclopropylmethyl, 1-2C-alkylcarbonyl, amidino, 2-fluoroethyl, 2,2,2-trifluoroethyl or 2,2-difluoroethyl, and
R1 is fluorine, or fluoromethyl.

One group of HetA radicals according to this second embodiment include HetA radicals substituted by R1, in which R1 is fluorine; such as, for example, 1N—(R10)-fluoro-pyrrolidinyl (e.g. 1N—(R10)-4-fluoro-pyrrolidin-3-yl), e.g. 1N-(1-3C-alkyl)-fluoro-pyrrolidinyl (like 1-methyl-4-fluoro-pyrrolidin-3-yl), or 1NH-fluoro-pyrrolidinyl (like 1H-4-fluoro-pyrrolidin-3-yl).

Another group of HetA radicals according to this second embodiment include HetA radicals substituted by R1, in which R1 is fluoromethyl; such as, for example, 1N—(R10)-fluoromethyl-pyrrolidinyl (e.g. 1N—(R10)-5-fluoromethyl-pyrrolidin-3-yl), e.g. 1N-(1-3C-alkyl)-fluoromethyl-pyrrolidinyl (like 1-methyl-5-fluoromethyl-pyrrolidin-3-yl).

In a third embodiment, HetA is tetrahydropyranyl or tetrahydrofuranyl.

Examplary HetA radicals according to this third embodiment include, without being restricted thereto, tetrahydropyran-4-yl.

HetB is optionally substituted by one or two substituents independently selected from 1-4C-alkyl and fluorine, and is piperidin-1-yl, morpholin-4-yl, thiomorpholin-4-yl, S-oxo-thiomorpholin-4-yl, S,S-dioxo-thiomorpholin-4-yl, pyrrolidin-1-yl, azetidin-1-yl, homopiperidin-1-yl, 4N—(R21)-piperazin-1-yl, 4N—(R21)-homopiperazin-1-yl, pyrrol-1-yl, pyrazol-1-yl, imidazol-1-yl, triazol-1-yl, or tetrazol-1-yl, in which
R21 is hydrogen, 1-4C-alkyl, 3-7C-cycloalkyl, 3-7C-cycloalkyl-1-4C-alkyl, 1-4C-alkylcarbonyl, amidino, or completely or partially fluorine-substituted 1-4C-alkyl,

in particular
R21 is hydrogen, 1-3C-alkyl, cyclopropyl, cyclopropylmethyl, 1-2C-alkylcarbonyl, or partially fluorine-substituted 1-3C-alkyl (e.g. 2-fluoroethyl, 2,2,2-trifluoroethyl or, particularly, 2,2-difluoroethyl).

In a first embodiment, HetB is piperidin-1-yl, morpholin-4-yl, pyrrolidin-1-yl or azetidin-1-yl.

In a second embodiment, HetB is 4N—(R21)-piperazin-1-yl, in which
R21 is hydrogen, methyl, ethyl, isopropyl, cyclopropyl, cyclopropylmethyl, 1-2C-alkylcarbonyl, 2-fluoroethyl, 2,2,2-trifluoroethyl or 2,2-difluoroethyl;
such as e.g. 4-methyl-piperazin-1-yl or 4-acetyl-piperazin-1-yl.

In a third embodiment, HetB is optionally substituted by one or two substituents independently selected from methyl and fluorine, and is piperidin-1-yl, pyrrolidin-1-yl, azetidin-1-yl or homopiperidin-1-yl; such as e.g. piperidin-1-yl, pyrrolidin-1-yl or azetidin-1-yl, or 4-methyl-piperidin-1-yl, 4-fluoro-piperidin-1-yl, 4,4-difluoro-piperidin-1-yl, (S)-3-fluoro-pyrrolidin-1-yl, (R)-3-fluoro-pyrrolidin-1-yl, 3,3-difluoro-pyrrolidin-1-yl, 3-fluoro-azetidin-1-yl or 3,3-difluoro-azetidin-1-yl.

In a fourth embodiment, HetB is pyrazol-1-yl, imidazol-1-yl or triazol-1-yl, especially imidazol-1-yl.

Amino-1-4C-alkyl denotes abovementioned 1-4C-alkyl radicals which are substituted by an amino group. Examples which may be mentioned are the aminomethyl, the 2-aminoethyl and the 3-aminopropyl radicals.

Hydroxy-2-4C-alkyl denotes abovementioned 2-4C-alkyl radicals which are substituted by a hydroxyl group. Examples which may be mentioned are the 2-hydroxyethyl and the 3-hydroxypropyl radicals.

1-4C-Alkoxy-2-4C-alkyl denotes abovementioned 2-4C-alkyl radicals which are substituted by one of the abovementioned 1-4C-alkoxy radicals. Examples which may be mentioned are the 2-methoxyethyl and the 3-methoxypropyl radicals.

Mono- or di-1-4C-alkylamino radicals contain, in addition to the nitrogen atom, one or two of the abovementioned 1-4C-alkyl radicals. Examples which may be mentioned are mono-1-4C-alkylamino radicals, like methylamino, ethylamino or isopropylamino, and di-1-4C-alkylamino radicals, like dimethylamino, diethylamino or diisopropylamino.

Mono- or di-1-4C-alkylamino-1-4C-alkyl represents one of the aforementioned 1-4C-alkyl groups, which is substituted by one of the aforementioned mono- or di-1-4C-alkylamino groups. Examples which may be mentioned are the methylamino-methyl, dimethylamino-methyl, 2-methylamino-ethyl, 2-dimethylamino-ethyl, 3-methylamino-propyl or 3-dimethylamino-propyl radicals.

1N—(R10)-piperidinyl or 1N—(R10)-pyrrolidinyl stands for a piperidinyl or pyrrolidinyl radical, respectively, which is substituted by R10 on the ring nitrogen atom in 1-position.

4N—(R21)-piperazin-1-yl or 4N—(R21)-homopiperazin-1-yl stands for a piperazin-1-yl or homopiperazin-1-yl radical, respectively, which is substituted by R21 on the ring nitrogen atom in 4-position.

The term (Raa)-methyl stands for methyl which is substituted by Raa. The term 2-(Raa)-ethyl stands for ethyl which is substituted in 2-position by Raa. The term 3-(Raa)-propyl stands for propyl which is substituted in 3-position by Raa. The term 4-(Raa)-butyl stands for butyl which is substituted in 4-position by Raa.

In general and unless otherwise mentioned, the heterocyclic radicals include all the possible isomeric forms thereof, e.g. the positional isomers thereof. Thus, for example, the term 1N—(R10)-piperidinyl includes 1N—(R10)-piperidin-2-yl and, particularly, 1N—(R10)-piperidin-3-yl and 1N—(R10)-piperidin-4-yl; or the term triazol-1-yl includes [1,2,3]triazol-1-yl and [1,2,4]triazol-1-yl.

Constituents which are optionally substituted as stated herein, may be substituted, unless otherwise noted, at any possible position.

Unless otherwise noted, the carbocyclic radicals mentioned herein may be substituted by its substituents or parent molecular groups at any possible position.

The heterocyclic groups mentioned herein may be substituted by their given substituents or parent molecular groups, unless otherwise noted, at any possible position, such as e.g. at any substitutable ring carbon or ring nitrogen atom.

Unless otherwise noted, rings containing quaternizable amino- or imino-type ring nitrogen atoms (—N═) may be preferably not quaternized on these amino- or imino-type ring nitrogen atoms by the mentioned substituents or parent molecular groups.

When any variable occurs more than one time in any constituent, each definition is independent.

Suitable salts for compounds according to this invention—depending on substitution—are all acid addition salts or all salts with bases. Particular mention may be made of the pharmacologically and/or pharmaceutically tolerable inorganic and organic acids and bases customarily used in pharmacy. Those suitable include, but are not limited to, water-insoluble and, particularly, water-soluble acid addition salts with acids such as, for example, hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, phenylsulphonic acid, methanesulphonic acid or 3-hydroxy-2-naphthoic acid, the acids being employed in salt preparation—depending on whether a mono- or polybasic acid is concerned and depending on which salt is desired—in an equimolar quantitative ratio or one differing therefrom.

Preferred are the salts selected from hydrochlorides, mesylates, tartrates, citrates, fumarates or sulfates.

On the other hand, salts with bases are—depending on substitution—also suitable. As examples of salts with bases are mentioned the lithium, sodium, potassium, calcium, aluminium, magnesium, titanium, ammonium, meglumine or guanidinium salts, here, too, the bases being employed in salt preparation in an equimolar quantitative ratio or one differing therefrom.

Salts which are unsuitable for pharmaceutical uses but which can be employed, for example, for the isolation or purification of free compounds of formula I or their pharmaceutically acceptable salts, are also included.

Pharmaceutically unacceptable salts, which can be obtained, for example, as process products during the preparation of the compounds according to this invention on an industrial scale, are converted into pharmaceutically acceptable salts by processes known to the person skilled in the art.

According to expert's knowledge the compounds of formula I according to this invention as well as their salts may contain, e.g. when isolated in crystalline form, varying amounts of solvents. Included within the scope of the invention are therefore all solvates and in particular all hydrates of the compounds of formula I according to this invention as well as all solvates and in particular all hydrates of the salts of the compounds of formula I according to this invention.

In one embodiment of this invention, salts of compounds of formula I include a salt of a compound of formula I with hydrochloric acid (hydrochloride).

In the context of this invention, hyperproliferation and analogous terms are used to describe aberrant/dysregulated cellular growth, a hallmark of diseases like cancer. This hyperproliferation might be caused by single or multiple cellular/molecular alterations in respective cells and can be, in context of a whole organism, of benign or malignant behaviour. Inhibition of cell proliferation and analogous terms is used herein to denote an ability of the compound to retard the growth of and/or kill a cell contacted with that compound as compared to cells not contacted with that compound. Most preferable this inhibition of cell proliferation is 100%, meaning that proliferation of all cells is stopped and/or cells undergo programmed cell death. In some preferred embodiments the contacted cell is a neoplastic cell. A neoplastic cell is defined as a cell with aberrant cell proliferation and/or the potential to metastasize to different tissues or organs. A benign neoplasia is described by hyperproliferation of cells, incapable of forming an aggressive, metastasizing tumor in-vivo. In contrast, a malignant neoplasia is described by cells with different cellular and biochemical abnormalities, e.g. capable of forming tumor metastasis. The aquired functional abnormalities of malignant neoplastic cells (also defined as “hallmarks of cancer”) are replicative potential (“hyperproliferation”), self-sufficiency in growth signals, insensitivity to anti-growth signals, evasion from apoptosis, sustained angiogenesis and tissue invasion and metastasis.

Inducer of apoptosis and analogous terms are used herein to identify a compound which executes programmed cell death in cells contacted with that compound. Apoptosis is defined by complex biochemical events within the contacted cell, such as the activation of cystein specific proteinases (“caspases”) and the fragmentation of chromatin. Induction of apoptosis in cells contacted with the compound might not necessarily be coupled with inhibition of cell proliferation. Preferably, the inhibition of cell proliferation and/or induction of apoptosis is specific to cells with aberrant cell growth (hyperproliferation). Thus, compared to cells with aberrant cell growth, normal proliferating or arrested cells are less sensitive or even insensitive to the proliferation inhibiting or apoptosis inducing activity of the compound. Finally, cytotoxic is used in a more general sense to identify compounds which kill cells by various mechanisms, including the induction of apoptosis/programmed cell death in a cell cycle dependent or cell-cycle independent manner.

Cell cycle specific and analogous terms are used herein to identify a compound as inducing apoptosis only in continously proliferating cells actively passing a specific phase of the cell cycle, but not in resting, non-dividing cells. Continously proliferating cells are typical for diseases like cancer and characterized by cells in all phases of the cell division cycle, namely in the G (“gap”) 1, S (“DNA synthesis”), G2 and M (“mitosis”) phase.

The invention especially relates to compounds of formula I, in which
Ra is 1-4C-alkyl, 3-7C-cycloalkyl, 3-7C-cycloalkyl-1-4C-alkyl, HetA, HetA-1-4C-alkyl, completely or partially fluorine-substituted 1-4C-alkyl, or 1-4C-alkyl substituted by Raa,
   wherein said 3-7C-cycloalkyl alone or as part of another group may be optionally substituted by one or two substituents independently selected from —N(R2)R3 and R1,

in which
Raa is —N(R2)R3, chlorine, bromine, hydroxyl, or 1-4C-alkoxy,
HetA is tetrahydropyranyl, tetrahydrofuranyl, 1N—(R10)-piperidinyl, or 1N—(R10)-pyrrolidinyl, in which
R10 is hydrogen, 1-4C-alkyl, 3-7C-cycloalkyl, 3-7C-cycloalkyl-1-4C-alkyl, 1-4C-alkylcarbonyl, amidino, or completely or partially fluorine-substituted 1-4C-alkyl,
wherein said HetA alone or as part of another group may be optionally substituted by one or two substituents independently selected from R1,
R1 is fluorine, or completely or partially fluorine-substituted 1-4C-alkyl,
R2 is hydrogen, 1-4C-alkyl, 3-7C-cycloalkyl, 3-7C-cycloalkyl-1-4C-alkyl, hydroxy-2-4C-alkyl, 1-4C-alkoxy-2-4C-alkyl, HetA, HetA-1-4C-alkyl, or completely or partially fluorine-substituted 1-4C-alkyl,
R3 is hydrogen, 1-4C-alkyl, 3-7C-cycloalkyl, or 3-7C-cycloalkyl-1-4C-alkyl,
or R2 and R3 together and with inclusion of the nitrogen atom, to which they are bonded, form a ring HetB, in which
HetB is piperidin-1-yl, morpholin-4-yl, thiomorpholin-4-yl, S-oxo-thiomorpholin-4-yl, S,S-dioxo-thiomorpholin-4-yl, pyrrolidin-1-yl, azetidin-1-yl, homopiperidin-1-yl, 4N—(R21)-piperazin-1-yl, 4N—(R21)-homopiperazin-1-yl, pyrrol-1-yl, pyrazol-1-yl, imidazol-1-yl, triazol-1-yl, or tetrazol-1-yl, in which
R21 is hydrogen, 1-4C-alkyl, 3-7C-cycloalkyl, 3-7C-cycloalkyl-1-4C-alkyl, 1-4C-alkylcarbonyl, amidino, or completely or partially fluorine-substituted 1-4C-alkyl,
wherein said HetB may be optionally substituted by one or two substituents independently selected from fluorine and 1-4C-alkyl,
Rb is 1-4C-alkyl, 3-7C-cycloalkyl, or 3-7C-cycloalkyl-1-4C-alkyl,
Rc is hydrogen or hydroxyl,
and the salts, stereoisomers and the salts of the stereoisomers of these compounds.

Compounds according to this invention worthy to be mentioned are those compounds of formula I, in which
Ra is 1-4C-alkyl, 3-6C-cycloalkyl, 3-6C-cycloalkyl-1-4C-alkyl, HetA, HetA-1-4C-alkyl, partially fluorine-substituted 1-3C-alkyl, or 2-4C-alkyl substituted by Raa,
   wherein said 3-6C-cycloalkyl alone or as part of another group may be optionally substituted by one or two substituents independently selected from —N(R2)R3 and R1,

in which
Raa is —N(R2)R3, chlorine, bromine, hydroxyl, or 1-3C-alkoxy,
HetA is tetrahydropyranyl, tetrahydrofuranyl, 1N—(R10)-piperidinyl, or 1N—(R10)-pyrrolidinyl, in which
R10 is hydrogen, 1-3C-alkyl, cyclopropyl, cyclopropylmethyl, 1-2C-alkylcarbonyl, amidino, or partially fluorine-substituted 1-3C-alkyl,
wherein said HetA alone or as part of another group may be optionally substituted by one or two substituents independently selected from R1,
R1 is fluorine, or completely or partially fluorine-substituted 1-2C-alkyl,
R2 is hydrogen, 1-4C-alkyl, cyclopropyl, cyclobutyl, cyclopropylmethyl, hydroxy-2-3C-alkyl, 1-3C-alkoxy-2-3C-alkyl, or partially fluorine-substituted 1-3C-alkyl,
R3 is hydrogen, 1-4C-alkyl, cyclopropyl, cyclobutyl, or cyclopropylmethyl,
or R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, homopiperidin-1-yl, 4N—(R21)-piperazin-1-yl, 4N—(R21)-homopiperazin-1-yl, pyrrol-1-yl, pyrazol-1-yl, imidazol-1-yl, triazol-1-yl, or tetrazol-1-yl, in which
R21 is hydrogen, 1-3C-alkyl, cyclopropyl, cyclopropylmethyl, 1-2C-alkylcarbonyl, amidino, or partially fluorine-substituted 1-3C-alkyl,
wherein said HetB may be optionally substituted by one or two substituents independently selected from fluorine and 1-3C-alkyl,
Rb is 1-4C-alkyl, 3-5C-cycloalkyl, or 3-5C-cycloalkyl-1-2C-alkyl,
Rc is hydrogen or hydroxyl,
and the salts, stereoisomers and the salts of the stereoisomers of these compounds.

Compounds according to this invention more worthy to be mentioned are those compounds of formula I, in which
Ra is 1-4C-alkyl, 3-6C-cycloalkyl, 3-6C-cycloalkyl-1-2C-alkyl, HetA, HetA-1-2C-alkyl, partially fluorine-substituted 2-3C-alkyl, or 2-3C-alkyl substituted by Raa,
   wherein said 3-6C-cycloalkyl alone or as part of another group may be optionally substituted by —N(R2)R3 and/or R1,

in which
Raa is —N(R2)R3, chlorine, or bromine,
HetA is tetrahydropyranyl, tetrahydrofuranyl, 1N—(R10)-piperidinyl, or 1N—(R10)-pyrrolidinyl, in which
R10 is hydrogen, 1-3C-alkyl, cyclopropyl, cyclopropylmethyl, 1-2C-alkylcarbonyl, amidino, or partially fluorine-substituted 2-3C-alkyl,
wherein said HetA alone or as part of another group may be optionally substituted by R1,
R1 is fluorine, or completely or partially fluorine-substituted 1-2C-alkyl,
R2 is hydrogen, 1-4C-alkyl, cyclopropyl, cyclobutyl, cyclopropylmethyl, 2-hydroxyethyl, 2-methoxyethyl, partially fluorine-substituted 2-3C-alkyl,
R3 is hydrogen, 1-4C-alkyl, cyclopropyl, cyclobutyl, or cyclopropylmethyl,
or R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, homopiperidin-1-yl, 4N—(R21)-piperazin-1-yl, 4N—(R21)-homopiperazin-1-yl, pyrazol-1-yl, imidazol-1-yl, triazol-1-yl, or tetrazol-1-yl, in which
R21 is hydrogen, 1-3C-alkyl, cyclopropyl, cyclopropylmethyl, 1-2C-alkylcarbonyl, amidino, or partially fluorine-substituted 2-3C-alkyl,
wherein said HetB may be optionally substituted by one or two substituents independently selected from fluorine and 1-2C-alkyl,
Rb is 1-4C-alkyl, cyclopropyl, cyclobutyl, or cyclopropylmethyl,
Rc is hydrogen or hydroxyl,
and the salts, stereoisomers and the salts of the stereoisomers of these compounds.

Compounds according to this invention further more worthy to be mentioned are those compounds of formula I, in which
Ra is 1-4C-alkyl, 5-6C-cycloalkyl, 5-6C-cycloalkyl-1-2C-alkyl, 3-4C-cycloalkyl, 3-4C-cycloalkyl-1-2C-alkyl, HetA, HetA-1-2C-alkyl, 2-fluoroethyl, 2,2-difluoroethyl, 2,2,2-trifluoroethyl, 2-(Raa)-ethyl, or 3-(Raa)-propyl,
   wherein said 5-6C-cycloalkyl alone or as part of another group may be optionally substituted by —N(R2)R3 and/or R1,

in which
Raa is —N(R2)R3, chlorine, or bromine,
HetA is tetrahydropyranyl, tetrahydrofuranyl, 1N—(R10)-piperidinyl, or 1N—(R10)-pyrrolidinyl, in which
R10 is hydrogen, 1-3C-alkyl, 1-2C-alkylcarbonyl, amidino, or partially fluorine-substituted 2-3C-alkyl,
wherein said HetA alone or as part of another group may be optionally substituted by R1,
R1 is fluorine, or completely or partially fluorine-substituted 1-2C-alkyl (particularly fluoromethyl),
R2 is hydrogen, methyl, ethyl, propyl, isopropyl, isobutyl, cyclopropyl, cyclobutyl, cyclopropylmethyl, 2-hydroxyethyl, 2-methoxyethyl, 2-fluoroethyl, 2,2-difluoroethyl, or 2,2,2-trifluoroethyl,
R3 is hydrogen, methyl, ethyl, propyl, isopropyl, or cyclopropyl,
or R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, homopiperidin-1-yl, 4N—(R21)-piperazin-1-yl, pyrazol-1-yl, imidazol-1-yl, or triazol-1-yl, in which
R21 is hydrogen, 1-3C-alkyl, 1-2C-alkylcarbonyl, or partially fluorine-substituted 2-3C-alkyl,
wherein said HetB may be optionally substituted by one or two substituents independently selected from fluorine and methyl,
Rb is methyl, ethyl, propyl, isopropyl, isobutyl, cyclopropyl, cyclobutyl, or cyclopropylmethyl,
Rc is hydrogen or hydroxyl,
and the salts, stereoisomers and the salts of the stereoisomers of these compounds.

Compounds according to this invention in particular worthy to be mentioned are those compounds of formula I, in which
Ra is 1-4C-alkyl, cyclohexyl, cyclopentyl, cyclopropyl, cyclobutyl, cyclopropylmethyl, HetA, HetA-methyl, 2-(Raa)-ethyl, or 3-(Raa)-propyl,
   wherein said cyclohexyl and cyclopentyl may be optionally substituted by —N(R2)R3 and/or R1,

in which
Raa is —N(R2)R3,
HetA is tetrahydropyranyl, tetrahydrofuranyl, 1N—(R10)-piperidinyl, or 1N—(R10)-pyrrolidinyl, in which
R10 is hydrogen, methyl, ethyl, propyl, isopropyl, acetyl, amidino, 2-fluoroethyl, 2,2-difluoroethyl, or 2,2,2-trifluoroethyl,
wherein said HetA alone or as part of another group may be optionally substituted by R1,
R1 is fluorine, or fluoromethyl,
either
R2 is hydrogen, and
R3 is hydrogen, or
R2 is methyl, ethyl, propyl, isopropyl, isobutyl, cyclopropyl, cyclobutyl, cyclopropylmethyl, 2-hydroxyethyl, 2-methoxyethyl, 2-fluoroethyl, 2,2-difluoroethyl, or 2,2,2-trifluoroethyl, and
R3 is hydrogen, or
R2 is methyl, ethyl, propyl, isopropyl, isobutyl, cyclopropyl, cyclobutyl, cyclopropylmethyl, 2-hydroxyethyl, 2-methoxyethyl, 2-fluoroethyl, 2,2-difluoroethyl, or 2,2,2-trifluoroethyl, and
R3 is methyl, or
R2 is ethyl, propyl, isopropyl, isobutyl, cyclopropyl, cyclobutyl, cyclopropylmethyl, 2-hydroxyethyl, 2-methoxyethyl, 2-fluoroethyl, 2,2-difluoroethyl, or 2,2,2-trifluoroethyl, and
R3 is ethyl, isopropyl, or cyclopropyl, or
R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, 4N—(R21)-piperazin-1-yl, pyrazol-1-yl, imidazol-1-yl, or triazol-1-yl, in which
R21 is hydrogen, methyl, ethyl, propyl, isopropyl, acetyl, 2-fluoroethyl, 2,2-difluoroethyl, or 2,2,2-trifluoroethyl,
wherein said HetB may be optionally substituted by one or two substituents independently selected from fluorine and methyl,
Rb is methyl, ethyl, isopropyl, or cyclopropyl,
Rc is hydrogen or hydroxyl,
and the salts, stereoisomers and the salts of the stereoisomers of these compounds.

Compounds according to this invention in more particular worthy to be mentioned are those compounds of formula I, in which
Ra is methyl, ethyl, propyl, isopropyl, isobutyl, cyclohexyl, cyclopentyl, cyclopropyl, cyclobutyl, cyclopropylmethyl, HetA, HetA-methyl, 2-(Raa)-ethyl, or 3-(Raa)-propyl,
   wherein said cyclohexyl and cyclopentyl may be optionally substituted by —N(R2)R3 and/or R1,

in which
Raa is —N(R2)R3,
HetA is tetrahydropyranyl, tetrahydrofuranyl, 1N—(R10)-piperidinyl, or 1N—(R10)-pyrrolidinyl, in which
R10 is hydrogen, methyl, ethyl, isopropyl, acetyl, amidino, 2-fluoroethyl, 2,2-difluoroethyl, or 2,2,2-trifluoroethyl,
wherein said HetA alone or as part of another group may be optionally substituted by R1,
R1 is fluorine, or fluoromethyl,
either
R2 is hydrogen, and
R3 is hydrogen, or
R2 is methyl, ethyl, propyl, isopropyl, isobutyl, cyclopropyl, cyclobutyl, cyclopropylmethyl, 2-hydroxyethyl, 2-methoxyethyl, 2-fluoroethyl, 2,2-difluoroethyl, or 2,2,2-trifluoroethyl, and
R3 is hydrogen, or
R2 is methyl, ethyl, propyl, isopropyl, isobutyl, cyclopropyl, cyclobutyl, cyclopropylmethyl, 2-hydroxyethyl, 2-methoxyethyl, 2-fluoroethyl, 2,2-difluoroethyl, or 2,2,2-trifluoroethyl, and
R3 is methyl, or
R2 is ethyl, propyl, isopropyl, cyclopropyl, 2-hydroxyethyl, 2-methoxyethyl, 2-fluoroethyl, 2,2-difluoroethyl, or 2,2,2-trifluoroethyl, and
R3 is ethyl, or
R2 and R3 together and with inclusion of the nitrogen atom, to which they are bonded, form a ring HetB, in which
either
HetB is piperidin-1-yl, morpholin-4-yl, pyrrolidin-1-yl, azetidin-1-yl, or 4N—(R21)-piperazin-1-yl, in which
R21 is hydrogen, methyl, ethyl, isopropyl, acetyl, 2-fluoroethyl, 2,2-difluoroethyl, or 2,2,2-trifluoroethyl,
wherein said HetB may be optionally substituted by one or two substituents independently selected from fluorine and methyl, or
HetB is pyrazol-1-yl, imidazol-1-yl, or triazol-1-yl,
Rb is methyl, ethyl, isopropyl, or cyclopropyl,
Rc is hydrogen,
and the salts, stereoisomers and the salts of the stereoisomers of these compounds.

Yet compounds according to this invention in more particular worthy to be mentioned are those compounds of formula I, in which
Ra is methyl, ethyl, propyl, isopropyl, isobutyl, cyclohexyl, cyclopentyl, cyclopropyl, cyclobutyl, cyclopropylmethyl, HetA, HetA-methyl, 2-(Raa)-ethyl, or 3-(Raa)-propyl,
   wherein said cyclohexyl and cyclopentyl may be optionally substituted by —N(R2)R3 and/or R1,

in which
Raa is —N(R2)R3,
HetA is tetrahydropyranyl, tetrahydrofuranyl, 1N—(R10)-piperidinyl, or 1N—(R10)-pyrrolidinyl, in which
R10 is hydrogen, methyl, ethyl, isopropyl, acetyl, amidino, 2-fluoroethyl, 2,2-difluoroethyl, or 2,2,2-trifluoroethyl,
wherein said HetA alone or as part of another group may be optionally substituted by R1,
R1 is fluorine, or fluoromethyl,
either
R2 is hydrogen, and
R3 is hydrogen, or
R2 is methyl, ethyl, propyl, isopropyl, isobutyl, cyclopropyl, cyclobutyl, cyclopropylmethyl, 2-hydroxyethyl, 2-methoxyethyl, 2-fluoroethyl, 2,2-difluoroethyl, or 2,2,2-trifluoroethyl, and
R3 is hydrogen, or
R2 is methyl, ethyl, propyl, isopropyl, isobutyl, cyclopropyl, cyclobutyl, cyclopropylmethyl, 2-hydroxyethyl, 2-methoxyethyl, 2-fluoroethyl, 2,2-difluoroethyl, or 2,2,2-trifluoroethyl, and
R3 is methyl, or
R2 is ethyl, propyl, isopropyl, cyclopropyl, 2-hydroxyethyl, 2-methoxyethyl, 2-fluoroethyl, 2,2-difluoroethyl, or 2,2,2-trifluoroethyl, and
R3 is ethyl, or
R2 and R3 together and with inclusion of the nitrogen atom, to which they are bonded, form a ring HetB, in which
either
HetB is piperidin-1-yl, morpholin-4-yl, pyrrolidin-1-yl, azetidin-1-yl, or 4N—(R21)-piperazin-1-yl, in which
R21 is hydrogen, methyl, ethyl, isopropyl, acetyl, 2-fluoroethyl, 2,2-difluoroethyl, or 2,2,2-trifluoroethyl,
wherein said HetB may be optionally substituted by one or two substituents independently selected from fluorine and methyl, or
HetB is pyrazol-1-yl, imidazol-1-yl, or triazol-1-yl,
Rb is methyl, ethyl, isopropyl, or cyclopropyl,
Rc is hydroxyl,
and the salts, stereoisomers and the salts of the stereoisomers of these compounds.

Further preferred compounds according to this invention worthy to be mentioned are those compounds of formula I, in which
Ra is methyl, ethyl, propyl, isopropyl, isobutyl, cyclohexyl, cyclopentyl, cyclopropyl, cyclobutyl, cyclopropylmethyl, HetA, HetA-methyl, 2-(Raa)-ethyl, or 3-(Raa)-propyl,
   wherein said cyclohexyl and cyclopentyl may be optionally substituted by —N(R2)R3 and/or R1,

in which
Raa is —N(R2)R3,
HetA is tetrahydropyranyl, tetrahydrofuranyl, 1N—(R10)-piperidinyl, or 1N—(R10)-pyrrolidinyl, in which
R10 is hydrogen, methyl, ethyl, isopropyl, acetyl, amidino, 2-fluoroethyl, 2,2-difluoroethyl, or 2,2,2-trifluoroethyl,
wherein said HetA alone or as part of another group may be optionally substituted by R1,
R1 is fluorine, or fluoromethyl,
either
R2 is hydrogen, and
R3 is hydrogen, or
R2 is methyl, ethyl, propyl, isopropyl, isobutyl, cyclopropyl, cyclobutyl, cyclopropylmethyl, 2-hydroxyethyl, 2-methoxyethyl, 2-fluoroethyl, 2,2-difluoroethyl, or 2,2,2-trifluoroethyl, and
R3 is hydrogen, or
R2 is methyl, ethyl, propyl, isopropyl, isobutyl, cyclopropyl, cyclobutyl, cyclopropylmethyl, 2-hydroxyethyl, 2-methoxyethyl, 2-fluoroethyl, 2,2-difluoroethyl, or 2,2,2-trifluoroethyl, and
R3 is methyl, or
R2 is ethyl, propyl, isopropyl, cyclopropyl, 2-hydroxyethyl, 2-methoxyethyl, 2-fluoroethyl, 2,2-difluoroethyl, or 2,2,2-trifluoroethyl, and
R3 is ethyl, or
R2 and R3 together and with inclusion of the nitrogen atom, to which they are bonded, form a ring HetB, in which
either
HetB is piperidin-1-yl, morpholin-4-yl, pyrrolidin-1-yl, azetidin-1-yl, or 4N—(R21)-piperazin-1-yl, in which
R21 is hydrogen, methyl, ethyl, isopropyl, acetyl, 2-fluoroethyl, 2,2-difluoroethyl, or 2,2,2-trifluoroethyl,
wherein said HetB may be optionally substituted by one or two substituents independently selected from fluorine and methyl, or
HetB is pyrazol-1-yl, imidazol-1-yl, or triazol-1-yl,
Rb is methyl, ethyl, isopropyl, or cyclopropyl,
Rc is hydrogen or hydroxyl,
and the salts, stereoisomers and the salts of the stereoisomers of these compounds.

Compounds according to this invention to be emphasized are those compounds of formula I, in which
Ra is methyl, ethyl, propyl, isopropyl, isobutyl, cyclohexyl, fluorocyclohexyl, cyclopentyl, fluorocyclopentyl, (fluoromethyl)cyclopentyl, cyclopropyl, cyclobutyl, cyclopropylmethyl, HetA, HetA-methyl, 2-(Raa)-ethyl, or 3-(Raa)-propyl,
   wherein said cyclohexyl, fluorocyclohexyl, cyclopentyl, fluorocyclopentyl and (fluoromethyl)cyclopentyl may be optionally substituted by —N(R2)R3,

in which
Raa is —N(R2)R3,
HetA is tetrahydropyranyl, tetrahydrofuranyl, fluorotetrahydropyranyl, fluorotetrahydrofuranyl, 1N—(R10)-piperidinyl, 1N—(R10)-fluoropiperidinyl, 1N—(R10)-pyrrolidinyl, 1N—(R10)-fluoropyrrolidinyl, 1N—(R10)-(fluoromethyl)pyrrolidinyl, or 1N—(R10)-(fluoromethyl)piperidinyl, in which
R10 is hydrogen, methyl, ethyl, isopropyl, acetyl, amidino, 2-fluoroethyl, 2,2-difluoroethyl, or 2,2,2-trifluoroethyl,
R1 is fluorine, or fluoromethyl,
either
R2 is hydrogen, and
R3 is hydrogen, or
R2 is methyl, ethyl, propyl, isopropyl, isobutyl, cyclopropyl, cyclobutyl, or cyclopropylmethyl, and
R3 is hydrogen, or
R2 is methyl, ethyl, isopropyl, or cyclopropyl, and
R3 is methyl, or
R2 is ethyl, isopropyl, or cyclopropyl, and
R3 is ethyl, or
R2 and R3 together and with inclusion of the nitrogen atom, to which they are bonded, form a ring HetB, in which
either
HetB is piperidin-1-yl, morpholin-4-yl, pyrrolidin-1-yl, azetidin-1-yl, 4N—(R21)-piperazin-1-yl, 4-methyl-piperidin-1-yl, 4-fluoro-piperidin-1-yl, 4,4-difluoro-piperidin-1-yl, (S)-3-fluoro-pyrrolidin-1-yl, (R)-3-fluoro-pyrrolidin-1-yl, 3,3-difluoro-pyrrolidin-1-yl, 3-fluoro-azetidin-1-yl, or 3,3-difluoro-azetidin-1-yl, in which
R21 is hydrogen, methyl, or acetyl, or
HetB is pyrazol-1-yl, or imidazol-1-yl,
Rb is methyl, ethyl, isopropyl, or cyclopropyl,
Rc is hydrogen,
and the salts, stereoisomers and the salts of the stereoisomers of these compounds.

Yet compounds according to this invention to be emphasized are those compounds of formula I, in which
Ra is methyl, ethyl, propyl, isopropyl, isobutyl, cyclohexyl, fluorocyclohexyl, cyclopentyl, fluorocyclopentyl, (fluoromethyl)cyclopentyl, cyclopropyl, cyclobutyl, cyclopropylmethyl, HetA, HetA-methyl, 2-(Raa)-ethyl, or 3-(Raa)-propyl,
   wherein said cyclohexyl, fluorocyclohexyl, cyclopentyl, fluorocyclopentyl and (fluoromethyl)cyclopentyl may be optionally substituted by —N(R2)R3,

in which
Raa is —N(R2)R3,
HetA is tetrahydropyranyl, tetrahydrofuranyl, fluorotetrahydropyranyl, fluorotetrahydrofuranyl, 1N—(R10)-piperidinyl, 1N—(R10)-fluoropiperidinyl, 1N—(R10)-pyrrolidinyl, 1N—(R10)-fluoropyrrolidinyl, 1N—(R10)-(fluoromethyl)pyrrolidinyl, or 1N—(R10)-(fluoromethyl)piperidinyl, in which
R10 is hydrogen, methyl, ethyl, isopropyl, acetyl, amidino, 2-fluoroethyl, 2,2-difluoroethyl, or 2,2,2-trifluoroethyl,
R1 is fluorine, or fluoromethyl,
either
R2 is hydrogen, and
R3 is hydrogen, or
R2 is methyl, ethyl, propyl, isopropyl, isobutyl, cyclopropyl, cyclobutyl, or cyclopropylmethyl, and
R3 is hydrogen, or
R2 is methyl, ethyl, isopropyl, or cyclopropyl, and
R3 is methyl, or
R2 is ethyl, isopropyl, or cyclopropyl, and
R3 is ethyl, or
R2 and R3 together and with inclusion of the nitrogen atom, to which they are bonded, form a ring HetB, in which
either
HetB is piperidin-1-yl, morpholin-4-yl, pyrrolidin-1-yl, azetidin-1-yl, 4N—(R21)-piperazin-1-yl, 4-methyl-piperidin-1-yl, 4-fluoro-piperidin-1-yl, 4,4-difluoro-piperidin-1-yl, (S)-3-fluoro-pyrrolidin-1-yl, (R)-3-fluoro-pyrrolidin-1-yl, 3,3-difluoro-pyrrolidin-1-yl, 3-fluoro-azetidin-1-yl, or 3,3-difluoro-azetidin-1-yl, in which
R21 is hydrogen, methyl, or acetyl, or
HetB is pyrazol-1-yl, or imidazol-1-yl,
Rb is methyl, ethyl, isopropyl, or cyclopropyl,
Rc is hydroxyl,
and the salts, stereoisomers and the salts of the stereoisomers of these compounds.

Compounds according to this invention to be more emphasized are those compounds of formula I, in which
Ra is methyl, ethyl, propyl, isopropyl, isobutyl, amino-cyclohexyl, HetA, HetA-methyl, 2-(Raa)-ethyl, or 3-(Raa)-propyl,

in which
Raa is —N(R2)R3,
either
HetA is tetrahydropyranyl, 1N—(R10)-piperidinyl, or 1N—(R10)-pyrrolidinyl, in which
R10 is hydrogen, methyl, ethyl, isopropyl, acetyl, 2-fluoroethyl, 2,2-difluoroethyl, or 2,2,2-trifluoroethyl, or
HetA is 1N—(R10)-fluoropiperidinyl, 1N—(R10)-fluoropyrrolidinyl, 1N—(R10)-(fluoromethyl)pyrrolidinyl, or 1N—(R10)-(fluoromethyl)piperidinyl,
   such as, for example, (3S,4R)-3-fluoro-1N—(R10)-piperidin-4-yl, (3R,4S)-3-fluoro-1N—(R10)-piperidin-4-yl, (3R,4R)-3-fluoro-1N—(R10)-piperidin-4-yl, (3S,4S)-3-fluoro-1N—(R10)-piperidin-4-yl, (2R,4R)-2-(fluoromethyl)-1N—(R10)-piperidin-4-yl, (2S,4S)-2-(fluoromethyl)-1N—(R10)-piperidin-4-yl, (3R,4R)-4-fluoro-1N—(R10)-pyrrolidin-3-yl, (3S,4S)-4-fluoro-1N—(R10)-pyrrolidin-3-yl, (3S,5R)-5-fluoromethyl-1N—(R10)-pyrrolidin-3-yl or (3S,5S)-5-fluoromethyl-1N—(R10)-pyrrolidin-3-yl, in which
R10 is hydrogen, methyl, ethyl, isopropyl, acetyl, 2-fluoroethyl, 2,2-difluoroethyl, or 2,2,2-trifluoroethyl,

in which
either
R2 is hydrogen, and
R3 is hydrogen, or
R2 is methyl, and
R3 is hydrogen, or
R2 is ethyl, and
R3 is hydrogen, or
R2 is methyl, and
R3 is methyl, or
R2 is ethyl, and
R3 is methyl, or
R2 is ethyl, and
R3 is ethyl, or
R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, 4-methyl-piperazin-1-yl, 4-acetyl-piperazin-1-yl, 4-methyl-piperidin-1-yl, 4-fluoro-piperidin-1-yl, 4,4-difluoro-piperidin-1-yl, (S)-3-fluoro-pyrrolidin-1-yl, (R)-3-fluoro-pyrrolidin-1-yl, 3,3-difluoro-pyrrolidin-1-yl, 3-fluoro-azetidin-1-yl, 3,3-difluoro-azetidin-1-yl, or imidazol-1-yl,
Rb is methyl, ethyl, isopropyl, or cyclopropyl,
Rc is hydrogen,
and the salts, stereoisomers and the salts of the stereoisomers of these compounds.

Yet compounds according to this invention to be more emphasized are those compounds of formula I,

in which
Ra is methyl, ethyl, propyl, isopropyl, isobutyl, amino-cyclohexyl, HetA, HetA-methyl, 2-(Raa)-ethyl, or 3-(Raa)-propyl,

in which
Raa is —N(R2)R3,
either
HetA is tetrahydropyranyl, 1N—(R10)-piperidinyl, or 1N—(R10)-pyrrolidinyl, in which
R10 is hydrogen, methyl, ethyl, isopropyl, acetyl, 2-fluoroethyl, 2,2-difluoroethyl, or 2,2,2-trifluoroethyl, or
HetA is 1N—(R10)-fluoropiperidinyl, 1N—(R10)-fluoropyrrolidinyl, 1N—(R10)-(fluoromethyl)pyrrolidinyl, or 1N—(R10)-(fluoromethyl)piperidinyl,
   such as, for example, (3S,4R)-3-fluoro-1N—(R10)-piperidin-4-yl, (3R,4S)-3-fluoro-1N—(R10)-piperidin-4-yl, (3R,4R)-3-fluoro-1N—(R10)-piperidin-4-yl, (3S,4S)-3-fluoro-1N—(R10)-piperidin-4-yl, (2R,4R)-2-(fluoromethyl)-1N—(R10)-piperidin-4-yl, (2S,4S)-2-(fluoromethyl)-1N—(R10)-piperidin-4-yl, (3R,4R)-4-fluoro-1N—(R10)-pyrrolidin-3-yl, (3S,4S)-4-fluoro-1N—(R10)-pyrrolidin-3-yl, (3S,5R)-5-fluoromethyl-1N—(R10)-pyrrolidin-3-yl or (3S,5S)-5-fluoromethyl-1N—(R10)-pyrrolidin-3-yl, in which
R10 is hydrogen, methyl, ethyl, isopropyl, acetyl, 2-fluoroethyl, 2,2-difluoroethyl, or 2,2,2-trifluoroethyl,

in which
either
R2 is hydrogen, and
R3 is hydrogen, or
R2 is methyl, and
R3 is hydrogen, or
R2 is ethyl, and
R3 is hydrogen, or
R2 is methyl, and
R3 is methyl, or
R2 is ethyl, and
R3 is methyl, or
R2 is ethyl, and
R3 is ethyl, or
R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, 4-methyl-piperazin-1-yl, 4-acetyl-piperazin-1-yl, 4-methyl-piperidin-1-yl, 4-fluoro-piperidin-1-yl, 4,4-difluoro-piperidin-1-yl, (S)-3-fluoro-pyrrolidin-1-yl, (R)-3-fluoro-pyrrolidin-1-yl, 3,3-difluoro-pyrrolidin-1-yl, 3-fluoro-azetidin-1-yl, 3,3-difluoro-azetidin-1-yl, or imidazol-1-yl,
Rb is methyl, ethyl, isopropyl, or cyclopropyl,
Rc is hydroxyl,
and the salts, stereoisomers and the salts of the stereoisomers of these compounds.

Compounds according to this invention to be emphasized are those compounds of formula I, in which
Ra is methyl, ethyl, propyl, isopropyl, isobutyl, amino-cyclohexyl, HetA, HetA-methyl, 2-(Raa)-ethyl, or 3-(Raa)-propyl,

in which
Raa is —N(R2)R3,
either
HetA is tetrahydropyranyl, 1N—(R10)-piperidinyl, or 1N—(R10)-pyrrolidinyl, in which
R10 is hydrogen, methyl, ethyl, isopropyl, acetyl, 2-fluoroethyl, 2,2-difluoroethyl, or 2,2,2-trifluoroethyl, or
HetA is 1N—(R10)-fluoropiperidinyl, 1N—(R10)-fluoropyrrolidinyl, 1N—(R10)-(fluoromethyl)pyrrolidinyl, or 1N—(R10)-(fluoromethyl)piperidinyl,
   such as, for example, (3S,4R)-3-fluoro-1N—(R10)-piperidin-4-yl, (3R,4S)-3-fluoro-1N—(R10)-piperidin-4-yl, (3R,4R)-3-fluoro-1N—(R10)-piperidin-4-yl, (3S,4S)-3-fluoro-1N—(R10)-piperidin-4-yl, (2R,4R)-2-(fluoromethyl)-1N—(R10)-piperidin-4-yl, (2S,4S)-2-(fluoromethyl)-1N—(R10)-piperidin-4-yl, (3R,4R)-4-fluoro-1N—(R10)-pyrrolidin-3-yl, (3S,4S)-4-fluoro-1N—(R10)-pyrrolidin-3-yl, (3S,5R)-5-fluoromethyl-1N—(R10)-pyrrolidin-3-yl or (3S,5S)-5-fluoromethyl-1N—(R10)-pyrrolidin-3-yl, in which
R10 is hydrogen, methyl, ethyl, isopropyl, acetyl, 2-fluoroethyl, 2,2-difluoroethyl, or 2,2,2-trifluoroethyl,

in which
either
R2 is hydrogen, and
R3 is hydrogen, or
R2 is methyl, and
R3 is hydrogen, or
R2 is ethyl, and
R3 is hydrogen, or
R2 is methyl, and
R3 is methyl, or
R2 is ethyl, and
R3 is methyl, or
R2 is ethyl, and
R3 is ethyl, or
R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, 4-methyl-piperazin-1-yl, 4-acetyl-piperazin-1-yl, 4-methyl-piperidin-1-yl, 4-fluoro-piperidin-1-yl, 4,4-difluoro-piperidin-1-yl, (S)-3-fluoro-pyrrolidin-1-yl, (R)-3-fluoro-pyrrolidin-1-yl, 3,3-difluoro-pyrrolidin-1-yl, 3-fluoro-azetidin-1-yl, 3,3-difluoro-azetidin-1-yl, or imidazol-1-yl,
Rb is methyl, ethyl, isopropyl, or cyclopropyl,
Rc is hydrogen or hydroxyl,
and the salts, stereoisomers and the salts of the stereoisomers of these compounds.

Compounds according to this invention to be further more emphasized are those compounds of formula I, in which
Ra is methyl, ethyl, propyl, isopropyl, isobutyl, 4-amino-cyclohexyl, HetA, HetA-methyl, 2-(Raa)-ethyl, or 3-(Raa)-propyl,

in which
Raa is —N(R2)R3,
either
HetA is tetrahydropyran-4-yl, 1N—(R10)-piperidin-3-yl, 1N—(R10)-piperidin-4-yl, or 1N—(R10)-pyrrolidin-3-yl, in which
R10 is hydrogen, methyl, ethyl, isopropyl, or 2,2-difluoroethyl, or
HetA is 3-fluoro-1N—(R10)-piperidin-4-yl, 4-fluoro-1N—(R10)-pyrrolidin-3-yl, 5-fluoromethyl-1N—(R10)-pyrrolidin-3-yl, or 2-(fluoromethyl)-1N—(R10)-piperidin-4-yl,
   such as, for example, (3S,4R)-3-fluoro-1N—(R10)-piperidin-4-yl, (3R,4S)-3-fluoro-1N—(R10)-piperidin-4-yl, (3R,4R)-3-fluoro-1N—(R10)-piperidin-4-yl, (3S,4S)-3-fluoro-1N—(R10)-piperidin-4-yl, (2R,4R)-2-(fluoromethyl)-1N—(R10)-piperidin-4-yl, (2S,4S)-2-(fluoromethyl)-1N—(R10)-piperidin-4-yl, (3R,4R)-4-fluoro-1N—(R10)-pyrrolidin-3-yl, (3S,4S)-4-fluoro-1N—(R10)-pyrrolidin-3-yl, (3S,5R)-5-fluoromethyl-1N—(R10)-pyrrolidin-3-yl or (3S,5S)-5-fluoromethyl-1N—(R10)-pyrrolidin-3-yl, in which
R10 is hydrogen, methyl, ethyl, or isopropyl,

in which
either
R2 is hydrogen, and
R3 is hydrogen, or
R2 is methyl, and
R3 is hydrogen, or
R2 is ethyl, and
R3 is hydrogen, or
R2 is methyl, and
R3 is methyl, or
R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, 4-methyl-piperazin-1-yl, or 4-acetyl-piperazin-1-yl,
Rb is methyl, ethyl, isopropyl, or cyclopropyl,
in particular,
Rb is methyl or ethyl,
in more particular,
Rb is methyl,
Rc is hydrogen,
and the salts, stereoisomers and the salts of the stereoisomers of these compounds.

Yet compounds according to this invention to be further more emphasized are those compounds of formula I, in which
Ra is methyl, ethyl, propyl, isopropyl, isobutyl, 4-amino-cyclohexyl, HetA, HetA-methyl, 2-(Raa)-ethyl, or 3-(Raa)-propyl,

in which
Raa is —N(R2)R3,
either
HetA is tetrahydropyran-4-yl, 1N—(R10)-piperidin-3-yl, 1N—(R10)-piperidin-4-yl, or 1N—(R10)-pyrrolidin-3-yl, in which
R10 is hydrogen, methyl, ethyl, isopropyl, or 2,2-difluoroethyl, or
HetA is 3-fluoro-1N—(R10)-piperidin-4-yl, 4-fluoro-1N—(R10)-pyrrolidin-3-yl, 5-fluoromethyl-1N—(R10)-pyrrolidin-3-yl, or 2-(fluoromethyl)-1N—(R10)-piperidin-4-yl,
   such as, for example, (3S,4R)-3-fluoro-1N—(R10)-piperidin-4-yl, (3R,4S)-3-fluoro-1N—(R10)-piperidin-4-yl, (3R,4R)-3-fluoro-1N—(R10)-piperidin-4-yl, (3S,4S)-3-fluoro-1N—(R10)-piperidin-4-yl, (2R,4R)-2-(fluoromethyl)-1N—(R10)-piperidin-4-yl, (2S,4S)-2-(fluoromethyl)-1N—(R10)-piperidin-4-yl, (3R,4R)-4-fluoro-1N—(R10)-pyrrolidin-3-yl, (3S,4S)-4-fluoro-1N—(R10)-pyrrolidin-3-yl, (3S,5R)-5-fluoromethyl-1N—(R10)-pyrrolidin-3-yl or (3S,5S)-5-fluoromethyl-1N—(R10)-pyrrolidin-3-yl, in which
R10 is hydrogen, methyl, ethyl, or isopropyl,

in which
either
R2 is hydrogen, and
R3 is hydrogen, or
R2 is methyl, and
R3 is hydrogen, or
R2 is ethyl, and
R3 is hydrogen, or
R2 is methyl, and
R3 is methyl, or
R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, 4-methyl-piperazin-1-yl, or 4-acetyl-piperazin-1-yl,
Rb is methyl, ethyl, isopropyl, or cyclopropyl,
in particular,
Rb is methyl or ethyl,
in more particular,
Rb is methyl,
Rc is hydroxyl,
and the salts, stereoisomers and the salts of the stereoisomers of these compounds.

Compounds according to this invention to be in particular emphasized are those compounds of formula I* or I** as shown later in this application, in which
Ra is methyl, ethyl, propyl, isopropyl, isobutyl, HetA, HetA-methyl, 2-(Raa)-ethyl, or 3-(Raa)-propyl,

in which
Raa is —N(R2)R3,
either
HetA is 1-methyl-piperidin-4-yl, 1H-piperidin-4-yl, 1-methyl-piperidin-3-yl, 1H-piperidin-3-yl, 1-isopropyl-piperidin-4-yl, or 1-isopropyl-piperidin-3-yl, or
HetA is (3S,4R)-3-fluoro-1-methyl-piperidin-4-yl, (3R,4S)-3-fluoro-1-methyl-piperidin-4-yl, (3R,4R)-3-fluoro-1-methyl-piperidin-4-yl, (3S,4S)-3-fluoro-1-methyl-piperidin-4-yl,
   (2R,4R)-2-(fluoromethyl)-1-methyl-piperidin-4-yl, (2S,4S)-2-(fluoromethyl)-1-methyl-piperidin-4-yl,
   (3R,4R)-4-fluoro-1-methyl-pyrrolidin-3-yl, (3S,4S)-4-fluoro-1-methyl-pyrrolidin-3-yl,
   (3R,4R)-4-fluoro-1H-pyrrolidin-3-yl, (3S,4S)-4-fluoro-1H-pyrrolidin-3-yl,
   (3S,5R)-5-fluoromethyl-1-methyl-pyrrolidin-3-yl, (3S,5S)-5-fluoromethyl-1-methyl-pyrrolidin-3-yl,
   (3S,5R)-5-fluoromethyl-1H-pyrrolidin-3-yl, or (3S,5S)-5-fluoromethyl-1H-pyrrolidin-3-yl,
HetA-methyl is (1-methyl-piperidin-4-yl)methyl, (1H-piperidin-4-yl)methyl, (1-methyl-piperidin-3-yl)methyl, (1H-piperidin-3-yl)methyl, (1-isopropyl-piperidin-4-yl)methyl, or (1-isopropyl-piperidin-3-yl)methyl,

in which
either
R2 is hydrogen, and
R3 is hydrogen, or
R2 is methyl, and
R3 is hydrogen, or
R2 is ethyl, and
R3 is hydrogen, or
R2 is methyl, and
R3 is methyl, or
R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, 4-methyl-piperazin-1-yl, or 4-acetyl-piperazin-1-yl,
Rb is methyl,
Rc is hydrogen,
and the salts, stereoisomers and the salts of the stereoisomers of these compounds.

Other compounds according to this invention to be in particular emphasized are those compounds of formula I* or I** as shown later in this application, in which
Ra is methyl, ethyl, propyl, isopropyl, isobutyl, HetA, HetA-methyl, 2-(Raa)-ethyl, or 3-(Raa)-propyl,

in which
Raa is —N(R2)R3,
either
HetA is 1-methyl-piperidin-4-yl, 1H-piperidin-4-yl, 1-methyl-piperidin-3-yl, 1H-piperidin-3-yl, 1-isopropyl-piperidin-4-yl, or 1-isopropyl-piperidin-3-yl, or
HetA is (3S,4R)-3-fluoro-1-methyl-piperidin-4-yl, (3R,4S)-3-fluoro-1-methyl-piperidin-4-yl, (3R,4R)-3-fluoro-1-methyl-piperidin-4-yl, (3S,4S)-3-fluoro-1-methyl-piperidin-4-yl,
   (2R,4R)-2-(fluoromethyl)-1-methyl-piperidin-4-yl, (2S,4S)-2-(fluoromethyl)-1-methyl-piperidin-4-yl,
   (3R,4R)-4-fluoro-1-methyl-pyrrolidin-3-yl, (3S,4S)-4-fluoro-1-methyl-pyrrolidin-3-yl,
   (3R,4R)-4-fluoro-1H-pyrrolidin-3-yl, (3S,4S)-4-fluoro-1H-pyrrolidin-3-yl,
   (3S,5R)-5-fluoromethyl-1-methyl-pyrrolidin-3-yl, (3S,5S)-5-fluoromethyl-1-methyl-pyrrolidin-3-yl,
   (3S,5R)-5-fluoromethyl-1H-pyrrolidin-3-yl, or (3S,5S)-5-fluoromethyl-1H-pyrrolidin-3-yl,
HetA-methyl is (1-methyl-piperidin-4-yl)methyl, (1H-piperidin-4-yl)methyl, (1-methyl-piperidin-3-yl)methyl, (1H-piperidin-3-yl)methyl, (1-isopropyl-piperidin-4-yl)methyl, or (1-isopropyl-piperidin-3-yl)methyl,

in which
either
R2 is hydrogen, and
R3 is hydrogen, or
R2 is methyl, and
R3 is hydrogen, or
R2 is ethyl, and
R3 is hydrogen, or
R2 is methyl, and
R3 is methyl, or
R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, 4-methyl-piperazin-1-yl, or 4-acetyl-piperazin-1-yl,
Rb is ethyl,
Rc is hydrogen,
and the salts, stereoisomers and the salts of the stereoisomers of these compounds.

Other compounds according to this invention to be in particular emphasized are those compounds of formula I* or I** as shown later in this application, in which
Ra is methyl, ethyl, propyl, isopropyl, isobutyl, HetA, HetA-methyl, 2-(Raa)-ethyl, or 3-(Raa)-propyl,

in which
Raa is —N(R2)R3,
either
HetA is 1-methyl-piperidin-4-yl, 1H-piperidin-4-yl, 1-methyl-piperidin-3-yl, 1H-piperidin-3-yl, 1-isopropyl-piperidin-4-yl, or 1-isopropyl-piperidin-3-yl, or
HetA is (3S,4R)-3-fluoro-1-methyl-piperidin-4-yl, (3R,4S)-3-fluoro-1-methyl-piperidin-4-yl, (3R,4R)-3-fluoro-1-methyl-piperidin-4-yl, (3S,4S)-3-fluoro-1-methyl-piperidin-4-yl,
   (2R,4R)-2-(fluoromethyl)-1-methyl-piperidin-4-yl, (2S,4S)-2-(fluoromethyl)-1-methyl-piperidin-4-yl,
   (3R,4R)-4-fluoro-1-methyl-pyrrolidin-3-yl, (3S,4S)-4-fluoro-1-methyl-pyrrolidin-3-yl,
   (3R,4R)-4-fluoro-1H-pyrrolidin-3-yl, (3S,4S)-4-fluoro-1H-pyrrolidin-3-yl,
   (3S,5R)-5-fluoromethyl-1-methyl-pyrrolidin-3-yl, (3S,5S)-5-fluoromethyl-1-methyl-pyrrolidin-3-yl,
   (3S,5R)-5-fluoromethyl-1H-pyrrolidin-3-yl, or (3S,5S)-5-fluoromethyl-1H-pyrrolidin-3-yl,
HetA-methyl is (1-methyl-piperidin-4-yl)methyl, (1H-piperidin-4-yl)methyl, (1-methyl-piperidin-3-yl)methyl, (1H-piperidin-3-yl)methyl, (1-isopropyl-piperidin-4-yl)methyl, or (1-isopropyl-piperidin-3-yl)methyl,

in which
either
R2 is hydrogen, and
R3 is hydrogen, or
R2 is methyl, and
R3 is hydrogen, or
R2 is ethyl, and
R3 is hydrogen, or
R2 is methyl, and
R3 is methyl, or
R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, 4-methyl-piperazin-1-yl, or 4-acetyl-piperazin-1-yl,
Rb is cyclopropyl,
Rc is hydrogen,
and the salts, stereoisomers and the salts of the stereoisomers of these compounds.

Yet compounds according to this invention to be in particular emphasized are those compounds of formula I* or I** as shown later in this application, in which
Ra is methyl, ethyl, propyl, isopropyl, isobutyl, HetA, HetA-methyl, 2-(Raa)-ethyl, or 3-(Raa)-propyl,

in which
Raa is —N(R2)R3,
either
HetA is 1-methyl-piperidin-4-yl, 1H-piperidin-4-yl, 1-methyl-piperidin-3-yl, 1H-piperidin-3-yl, 1-isopropyl-piperidin-4-yl, or 1-isopropyl-piperidin-3-yl, or
HetA is (3S,4R)-3-fluoro-1-methyl-piperidin-4-yl, (3R,4S)-3-fluoro-1-methyl-piperidin-4-yl, (3R,4R)-3-fluoro-1-methyl-piperidin-4-yl, (3S,4S)-3-fluoro-1-methyl-piperidin-4-yl,
   (2R,4R)-2-(fluoromethyl)-1-methyl-piperidin-4-yl, (2S,4S)-2-(fluoromethyl)-1-methyl-piperidin-4-yl,
   (3R,4R)-4-fluoro-1-methyl-pyrrolidin-3-yl, (3S,4S)-4-fluoro-1-methyl-pyrrolidin-3-yl,
   (3R,4R)-4-fluoro-1H-pyrrolidin-3-yl, (3S,4S)-4-fluoro-1H-pyrrolidin-3-yl,
   (3S,5R)-5-fluoromethyl-1-methyl-pyrrolidin-3-yl, (3S,5S)-5-fluoromethyl-1-methyl-pyrrolidin-3-yl,
   (3S,5R)-5-fluoromethyl-1H-pyrrolidin-3-yl, or (3S,5S)-5-fluoromethyl-1H-pyrrolidin-3-yl,
HetA-methyl is (1-methyl-piperidin-4-yl)methyl, (1H-piperidin-4-yl)methyl, (1-methyl-piperidin-3-yl)methyl, (1H-piperidin-3-yl)methyl, (1-isopropyl-piperidin-4-yl)methyl, or (1-isopropyl-piperidin-3-yl)methyl,

in which
either
R2 is hydrogen, and
R3 is hydrogen, or
R2 is methyl, and
R3 is hydrogen, or
R2 is ethyl, and
R3 is hydrogen, or
R2 is methyl, and
R3 is methyl, or
R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, 4-methyl-piperazin-1-yl, or 4-acetyl-piperazin-1-yl,
Rb is methyl,
Rc is hydroxyl,
and the salts, stereoisomers and the salts of the stereoisomers of these compounds.

Yet other compounds according to this invention to be in particular emphasized are those compounds of formula I* or I** as shown later in this application, in which
Ra is methyl, ethyl, propyl, isopropyl, isobutyl, HetA, HetA-methyl, 2-(Raa)-ethyl, or 3-(Raa)-propyl,

in which
Raa is —N(R2)R3,
either
HetA is 1-methyl-piperidin-4-yl, 1H-piperidin-4-yl, 1-methyl-piperidin-3-yl, 1H-piperidin-3-yl, 1-isopropyl-piperidin-4-yl, or 1-isopropyl-piperidin-3-yl, or
HetA is (3S,4R)-3-fluoro-1-methyl-piperidin-4-yl, (3R,4S)-3-fluoro-1-methyl-piperidin-4-yl, (3R,4R)-3-fluoro-1-methyl-piperidin-4-yl, (3S,4S)-3-fluoro-1-methyl-piperidin-4-yl,
   (2R,4R)-2-(fluoromethyl)-1-methyl-piperidin-4-yl, (2S,4S)-2-(fluoromethyl)-1-methyl-piperidin-4-yl,
   (3R,4R)-4-fluoro-1-methyl-pyrrolidin-3-yl, (3S,4S)-4-fluoro-1-methyl-pyrrolidin-3-yl,
   (3R,4R)-4-fluoro-1H-pyrrolidin-3-yl, (3S,4S)-4-fluoro-1H-pyrrolidin-3-yl,
   (3S,5R)-5-fluoromethyl-1-methyl-pyrrolidin-3-yl, (3S,5S)-5-fluoromethyl-1-methyl-pyrrolidin-3-yl,
   (3S,5R)-5-fluoromethyl-1H-pyrrolidin-3-yl, or (3S,5S)-5-fluoromethyl-1H-pyrrolidin-3-yl,
HetA-methyl is (1-methyl-piperidin-4-yl)methyl, (1H-piperidin-4-yl)methyl, (1-methyl-piperidin-3-yl)methyl, (1H-piperidin-3-yl)methyl, (1-isopropyl-piperidin-4-yl)methyl, or (1-isopropyl-piperidin-3-yl)methyl,

in which
either
R2 is hydrogen, and
R3 is hydrogen, or
R2 is methyl, and
R3 is hydrogen, or
R2 is ethyl, and
R3 is hydrogen, or
R2 is methyl, and
R3 is methyl, or
R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, 4-methyl-piperazin-1-yl, or 4-acetyl-piperazin-1-yl,
Rb is ethyl,
Rc is hydroxyl,
and the salts, stereoisomers and the salts of the stereoisomers of these compounds.

Yet other compounds according to this invention to be in particular emphasized are those compounds of formula I* or I** as shown later in this application, in which
Ra is methyl, ethyl, propyl, isopropyl, isobutyl, HetA, HetA-methyl, 2-(Raa)-ethyl, or 3-(Raa)-propyl,

in which
Raa is —N(R2)R3,
either
HetA is 1-methyl-piperidin-4-yl, 1H-piperidin-4-yl, 1-methyl-piperidin-3-yl, 1H-piperidin-3-yl, 1-isopropyl-piperidin-4-yl, or 1-isopropyl-piperidin-3-yl, or
HetA is (3S,4R)-3-fluoro-1-methyl-piperidin-4-yl, (3R,4S)-3-fluoro-1-methyl-piperidin-4-yl, (3R,4R)-3-fluoro-1-methyl-piperidin-4-yl, (3S,4S)-3-fluoro-1-methyl-piperidin-4-yl,
   (2R,4R)-2-(fluoromethyl)-1-methyl-piperidin-4-yl, (2S,4S)-2-(fluoromethyl)-1-methyl-piperidin-4-yl,
   (3R,4R)-4-fluoro-1-methyl-pyrrolidin-3-yl, (3S,4S)-4-fluoro-1-methyl-pyrrolidin-3-yl,
   (3R,4R)-4-fluoro-1H-pyrrolidin-3-yl, (3S,4S)-4-fluoro-1H-pyrrolidin-3-yl,
   (3S,5R)-5-fluoromethyl-1-methyl-pyrrolidin-3-yl, (3S,5S)-5-fluoromethyl-1-methyl-pyrrolidin-3-yl,
   (3S,5R)-5-fluoromethyl-1H-pyrrolidin-3-yl, or (3S,5S)-5-fluoromethyl-1H-pyrrolidin-3-yl,
HetA-methyl is (1-methyl-piperidin-4-yl)methyl, (1H-piperidin-4-yl)methyl, (1-methyl-piperidin-3-yl)methyl, (1H-piperidin-3-yl)methyl, (1-isopropyl-piperidin-4-yl)methyl, or (1-isopropyl-piperidin-3-yl)methyl,

in which
either
R2 is hydrogen, and
R3 is hydrogen, or
R2 is methyl, and
R3 is hydrogen, or
R2 is ethyl, and
R3 is hydrogen, or
R2 is methyl, and
R3 is methyl, or
R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, 4-methyl-piperazin-1-yl, or 4-acetyl-piperazin-1-yl,
Rb is cyclopropyl,
Rc is hydroxyl,
and the salts, stereoisomers and the salts of the stereoisomers of these compounds.

Compounds according to this invention to be in more particular emphasized are those compounds of formula I* or I** as shown later in this application, in which
Ra is methyl, ethyl, propyl, isopropyl, isobutyl, HetA, HetA-methyl, 2-(Raa)-ethyl, or 3-(Raa)-propyl,

in which
Raa is —N(R2)R3,
HetA is 1-methyl-piperidin-4-yl, 1H-piperidin-4-yl, 1-methyl-piperidin-3-yl, 1H-piperidin-3-yl, 1-isopropyl-piperidin-4-yl, or 1-isopropyl-piperidin-3-yl,

in which
either
R2 is hydrogen, and
R3 is hydrogen, or
R2 is methyl, and
R3 is hydrogen, or
R2 is ethyl, and
R3 is hydrogen, or
R2 is methyl, and
R3 is methyl, or
R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, 4-methyl-piperazin-1-yl, or 4-acetyl-piperazin-1-yl,
Rb is methyl, ethyl, or cyclopropyl,
in particular,
Rb is methyl or ethyl,
in more particular,
Rb is methyl,
Rc is hydrogen,
and the salts, stereoisomers and the salts of the stereoisomers of these compounds.

Yet compounds according to this invention to be in more particular emphasized are those compounds of formula I* or I** as shown later in this application, in which
Ra is methyl, ethyl, propyl, isopropyl, isobutyl, HetA, HetA-methyl, 2-(Raa)-ethyl, or 3-(Raa)-propyl,

in which
Raa is —N(R2)R3,
HetA is 1-methyl-piperidin-4-yl, 1H-piperidin-4-yl, 1-methyl-piperidin-3-yl, 1H-piperidin-3-yl, 1-isopropyl-piperidin-4-yl, or 1-isopropyl-piperidin-3-yl,

in which
either
R2 is hydrogen, and
R3 is hydrogen, or
R2 is methyl, and
R3 is hydrogen, or
R2 is ethyl, and
R3 is hydrogen, or
R2 is methyl, and
R3 is methyl, or
R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, 4-methyl-piperazin-1-yl, or 4-acetyl-piperazin-1-yl,
Rb is methyl, ethyl, or cyclopropyl,
in particular,
Rb is methyl or ethyl,
in more particular,
Rb is methyl,
Rc is hydroxyl,
and the salts, stereoisomers and the salts of the stereoisomers of these compounds.

The invention particularly relates to compounds of formula I according to the invention, in which
Ra is 1-4C-alkyl, HetA, HetA-1-4C-alkyl, or 1-4C-alkyl substituted by Raa,

in which
Raa is —N(R2)R3,
HetA is 1N—(R10)-piperidinyl, in which
R10 is hydrogen, 1-4C-alkyl, or 1-4C-alkylcarbonyl,
R2 is hydrogen or 1-4C-alkyl,
R3 is hydrogen or 1-4C-alkyl,
or R2 and R3 together and with inclusion of the nitrogen atom, to which they are bonded, form a ring HetB, in which
HetB is 4N—(R21)-piperazin-1-yl, or pyrrol-1-yl, in which
R21 is 1-4C-alkylcarbonyl,
Rb is 1-4C-alkyl or 3-7C-cycloalkyl,
Rc is hydrogen or hydroxyl,
and the salts, stereoisomers and the salts of the stereoisomers of these compounds.

The invention further relates particularly to compounds of formula I according to the invention, in which
Ra is 1-4C-alkyl, HetA, HetA-1-4C-alkyl, or 1-4C-alkyl substituted by Raa,

in which
Raa is —N(R2)R3 or chlorine,
HetA is 1N—(R10)-piperidinyl, in which
R10 is hydrogen, 1-4C-alkyl, or 1-4C-alkylcarbonyl,
R2 is hydrogen, 1-4C-alkyl, 3-7C-cycloalkyl, 3-7C-cycloalkyl-1-4C-alkyl, 2-4C-alkenyl, 2-4C-alkynyl, 1-4C-alkoxycarbonyl, hydroxy-2-4C-alkyl, 1-4C-alkoxy-2-4C-alkyl, or completely or partially fluorine-substituted 1-4C-alkyl,
R3 is hydrogen, 1-4C-alkyl,
or R2 and R3 together and with inclusion of the nitrogen atom, to which they are bonded, form a ring HetB, in which
HetB is piperidin-1-yl, azetidin-1-yl, 4N—(R21)-piperazin-1-yl, or pyrrol-1-yl, in which
R21 is 1-4C-alkylcarbonyl,
wherein said HetB may be optionally substituted by 1-4C-alkyl,
Rb is 1-4C-alkyl or 3-7C-cycloalkyl,
Rc is hydrogen or hydroxyl,
and the salts, stereoisomers and the salts of the stereoisomers of these compounds.

Most preferred are compounds of formula I according to the invention, in which
Ra is HetA or 1-4C-alkyl substituted by Raa,

in which
Raa is —N(R2)R3,
HetA is 1N—(R10)-piperidinyl, in which
R10 is hydrogen or methyl
R2 1-4C-alkyl or hydroxy-2-4C-alkyl,
R3 is hydrogen, 1-4C-alkyl,
or R2 and R3 together and with inclusion of the nitrogen atom, to which they are bonded, form a ring HetB, in which
HetB is piperidin-1-yl, azetidin-1-yl, or pyrrol-1-yl, in which
Rb is 1-4C-alkyl or 3-7C-cycloalkyl,
Rc is hydrogen or hydroxyl,
and the salts, stereoisomers and the salts of the stereoisomers of these compounds.

A special interest in the compounds according to this invention refers to those compounds of formula I which are included—within the scope of this invention—by one or, when possible, by a combination of more of the following special embodiments:

A special embodiment (embodiment 1) of the compounds of formula I according to this invention refers to those compounds of formula I, in which
Ra is methyl, ethyl, propyl, isopropyl or isobutyl,
Rb is methyl, ethyl, isopropyl or cyclopropyl, and
Rc is hydrogen.

Another special embodiment (embodiment 2) of the compounds of formula I according to this invention refers to those compounds of formula I, in which
Ra is 1N—(R10)-piperidin-3-yl or 1N—(R10)-piperidin-4-yl, in which
R10 is hydrogen, methyl, ethyl, isopropyl or cyclopropyl,
Rb is methyl, ethyl, isopropyl or cyclopropyl,
Rc is hydrogen.

Another special embodiment (embodiment 3) of the compounds of formula I according to this invention refers to those compounds of formula I, in which
Ra is 1N—(R10)-piperidin-3-yl-methyl or 1N—(R10)-piperidin-4-yl-methyl, in which
R10 is hydrogen, methyl, ethyl, isopropyl or cyclopropyl,
Rb is methyl, ethyl, isopropyl or cyclopropyl, and
Rc is hydrogen.

Another special embodiment (embodiment 4) of the compounds of formula I according to this invention refers to those compounds of formula I, in which
Ra is 2-aminoethyl or 3-aminopropyl,
Rb is methyl, ethyl, isopropyl or cyclopropyl, and
Rc is hydrogen.

Another special embodiment (embodiment 5) of the compounds of formula I according to this invention refers to those compounds of formula I, in which
Ra is 2-methylamino-ethyl, 2-dimethylamino-ethyl, 3-methylamino-propyl or 3-dimethylamino-propyl,
Rb is methyl, ethyl, isopropyl or cyclopropyl, and
Rc is hydrogen.

Another special embodiment (embodiment 6) of the compounds of formula I according to this invention refers to those compounds of formula I, in which
Ra is 2-pyrrolidin-1-yl-ethyl, 2-piperidin-1-yl-ethyl, 2-azetidin-1-yl-ethyl, 3-pyrrolidin-1-yl-propyl, 3-piperidin-1-yl-propyl or 3-azetidin-1-yl-propyl,
Rb is methyl, ethyl, isopropyl or cyclopropyl, and
Rc is hydrogen.

Another special embodiment (embodiment 7) of the compounds of formula I according to this invention refers to those compounds of formula I, in which
Ra is methyl, ethyl, propyl, isopropyl or isobutyl,
Rb is methyl, ethyl, isopropyl or cyclopropyl, and
Rc is hydroxyl.

Another special embodiment (embodiment 8) of the compounds of formula I according to this invention refers to those compounds of formula I, in which
Ra is 1N—(R10)-piperidin-3-yl or 1N—(R10)-piperidin-4-yl, in which
R10 is hydrogen, methyl, ethyl, isopropyl or cyclopropyl,
Rb is methyl, ethyl, isopropyl or cyclopropyl,
Rc is hydroxyl.

Another special embodiment (embodiment 9) of the compounds of formula I according to this invention refers to those compounds of formula I, in which
Ra is 1N—(R10)-piperidin-3-yl-methyl or 1N—(R10)-piperidin-4-yl-methyl, in which
R10 is hydrogen, methyl, ethyl, isopropyl or cyclopropyl,
Rb is methyl, ethyl, isopropyl or cyclopropyl, and
Rc is hydroxyl.

Another special embodiment (embodiment 10) of the compounds of formula I according to this invention refers to those compounds of formula I, in which
Ra is 2-aminoethyl or 3-aminopropyl,
Rb is methyl, ethyl, isopropyl or cyclopropyl, and
Rc is hydroxyl.

Another special embodiment (embodiment 11) of the compounds of formula I according to this invention refers to those compounds of formula I, in which
Ra is 2-methylamino-ethyl, 2-dimethylamino-ethyl, 3-methylamino-propyl or 3-dimethylamino-propyl,
Rb is methyl, ethyl, isopropyl or cyclopropyl, and
Rc is hydroxyl.

Another special embodiment (embodiment 12) of the compounds of formula I according to this invention refers to those compounds of formula I, in which
Ra is 2-pyrrolidin-1-yl-ethyl, 2-piperidin-1-yl-ethyl, 2-azetidin-1-yl-ethyl, 3-pyrrolidin-1-yl-propyl, 3-piperidin-1-yl-propyl or 3-azetidin-1-yl-propyl,
Rb is methyl, ethyl, isopropyl or cyclopropyl, and
Rc is hydroxyl.

Another special embodiment (embodiment 13) of the compounds of formula I according to this invention refers to those compounds of formula I, in which
Rb is methyl.

Another special embodiment (embodiment 14) of the compounds of formula I according to this invention refers to those compounds of formula I, in which
Rb is ethyl.

Another special embodiment (embodiment 15) of the compounds of formula I according to this invention refers to those compounds of formula I, in which
Rb is isopropyl.

Another special embodiment (embodiment 16) of the compounds of formula I according to this invention refers to those compounds of formula I, in which
Rb is cyclopropyl.

Another special embodiment (embodiment 17) of the compounds of formula I according to this invention refers to those compounds which are from formula I* as shown below, in which
Rc is hydrogen.

Another special embodiment (embodiment 18) of the compounds of formula I according to this invention refers to those compounds which are from formula I** as shown below, in which
Rc is hydrogen.

Another special embodiment (embodiment 19) of the compounds of formula I according to this invention refers to those compounds which are from formula I* as shown below, in which
Rc is hydroxyl.

Another special embodiment (embodiment 20) of the compounds of formula I according to this invention refers to those compounds which are from formula I** as shown below, in which
Rc is hydroxyl.

Another special embodiment (embodiment 21) of the compounds of formula I according to this invention refers to those compounds which are from formula I* as shown below, in which
Rb is methyl, and
Rc is hydrogen.

Another special embodiment (embodiment 22) of the compounds of formula I according to this invention refers to those compounds which are from formula I* as shown below, in which
Rb is ethyl, and
Rc is hydrogen.

Another special embodiment (embodiment 23) of the compounds of formula I according to this invention refers to those compounds which are from formula I* as shown below, in which
Rb is isopropyl, and
Rc is hydrogen.

Another special embodiment (embodiment 24) of the compounds of formula I according to this invention refers to those compounds which are from formula I* as shown below, in which
Rb is cyclopropyl, and
Rc is hydrogen.

Another special embodiment (embodiment 25) of the compounds of formula I according to this invention refers to those compounds which are from formula I* as shown below, in which
Rb is methyl, and
Rc is hydroxyl.

Another special embodiment (embodiment 26) of the compounds of formula I according to this invention refers to those compounds which are from formula I* as shown below, in which
Rb is ethyl, and
Rc is hydroxyl.

Another special embodiment (embodiment 27) of the compounds of formula I according to this invention refers to those compounds which are from formula I* as shown below, in which
Rb is isopropyl, and
Rc is hydroxyl.

Another special embodiment (embodiment 28) of the compounds of formula I according to this invention refers to those compounds which are from formula I* as shown below, in which
Rb is cyclopropyl, and
Rc is hydroxyl.

Another special embodiment (embodiment 29) of the compounds of formula I according to this invention refers to those compounds which are from formula I** as shown below, in which
Rb is methyl, and
Rc is hydrogen.

Another special embodiment (embodiment 30) of the compounds of formula I according to this invention refers to those compounds which are from formula I** as shown below, in which
Rb is ethyl, and
Rc is hydrogen.

Another special embodiment (embodiment 31) of the compounds of formula I according to this invention refers to those compounds which are from formula I** as shown below, in which
Rb is isopropyl, and
Rc is hydrogen.

Another special embodiment (embodiment 32) of the compounds of formula I according to this invention refers to those compounds which are from formula I** as shown below, in which
Rb is cyclopropyl, and
Rc is hydrogen.

Another special embodiment (embodiment 33) of the compounds of formula I according to this invention refers to those compounds which are from formula I** as shown below, in which
Rb is methyl, and
Rc is hydroxyl.

Another special embodiment (embodiment 34) of the compounds of formula I according to this invention refers to those compounds which are from formula I** as shown below, in which
Rb is ethyl, and
Rc is hydroxyl.

Another special embodiment (embodiment 35) of the compounds of formula I according to this invention refers to those compounds which are from formula I** as shown below, in which
Rb is isopropyl, and
Rc is hydroxyl.

Another special embodiment (embodiment 36) of the compounds of formula I according to this invention refers to those compounds which are from formula I** as shown below, in which
Rb is cyclopropyl, and
Rc is hydroxyl.

Another special embodiment (embodiment 37) of the compounds of formula I according to this invention refers to those compounds which are from formula I** as shown below, in which
Ra is 2-(Raa)-ethyl, or 3-(Raa)-propyl,
Raa is —N(R2)R3,
R2 is 2-4C-alkenyl or 2-4C-alkynyl,
R3 is hydrogen, 1-4C-alkyl,
Rb is methyl,
Rc is hydrogen or hydroxyl.

Another special embodiment (embodiment 38) of the compounds of formula I according to this invention refers to those compounds which are from formula I** as shown below, in which
Ra is 2-(Raa)-ethyl, or 3-(Raa)-propyl, in which
Raa is —N(R2)R3,
R2 is 1-4C-alkoxycarbonyl,
R3 is hydrogen, 1-4C-alkyl,
Rb is methyl,
Rc is hydrogen or hydroxyl.

Another special embodiment (embodiment 39) of the compounds of formula I according to this invention refers to those compounds which are from formula I** as shown below, in which
Ra is 1N—(R10)-piperidin-3-yl or 1N—(R10)-piperidin-4-yl, in which
R10 is hydrogen, methyl, 1-4C-alkylcarbonyl, or 1-4C-alkoxycarbonyl,
Rb is methyl,
Rc is hydrogen or hydroxyl.

It is to be understood that the present invention includes any or all possible combinations and subsets of the special embodiments defined hereinabove.

The compounds of formula I are chiral compounds having chiral centers at least in positions 3 and 3a.

### Numbering

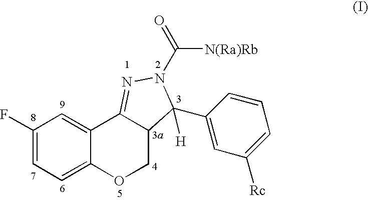

The invention includes all conceivable stereoisomers of the compounds of this invention, like e.g. diastereomers and enantiomers, in substantially pure form as well as in any mixing ratio, including the racemates, as well as the salts thereof.

Thus, substantially pure stereoisomers of the compounds according to this invention, particularly substantially pure stereoisomers of the following examples, are all part of the present invention and may be obtained according to procedures customary to the skilled person, e.g. by separation of corresponding mixtures, by using stereochemically pure starting materials and/or by stereoselective synthesis.

Emphasis is given to compounds of formula I, which have, with respect to the position 3, the same configuration as shown in formula Ia:

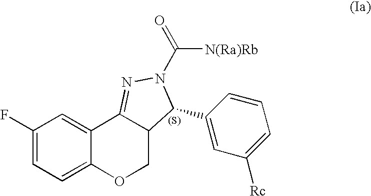

If, for example, in compounds of formula Ia Ra, Rb and Rc have the meanings given above, then the configuration—according to the rules of Cahn, Ingold and Prelog—is S in the 3 position. The individual diastereomers having (3S) configuration both in pure and in mixture form, as well as the salts, stereoisomers and salts of the stereoisomers thereof, are particularly worthy to be mentioned in this context.

The hydrogen atoms in positions 3 and 3a can be arranged in cis or in trans position relative to one another. Worthy to be mentioned are hereby those compounds of formula I in which the hydrogen atoms in positions 3 and 3a are in the cis position relative to one another. The pure cis enantiomers and their mixtures in any mixing ratio, including the racemates, as well as the salts thereof, are more worthy to be mentioned in this context.

Further on, compounds of formula I and the salts thereof include stereoisomers. Each of the stereogenic centers present in said stereoisomers may have the absolute configuration R or the absolute configuration S (according to the rules of Cahn, Ingold and Prelog). Accordingly, the stereoisomers (3R,3aR), (3R,3aS), (3S,3aR) and (3S,3aS), wherein the numbers refer to the atoms indicated in formula I above, and the salts thereof are part of the invention. From these, the stereosiomers (3S,3aR) and, especially, (3S,3aS) as well as the salts thereof are more worthy to be noted.

In this connection, compounds of formula I in particular worthy to be noted are those which have, with respect to the positions 3 and 3a, the same configuration as shown in formula I*:

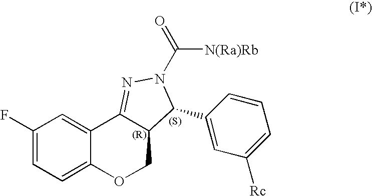

If, for example, in compounds of formula I* Ra, Rb and Rc have the meanings given above, then the configuration—according to the rules of Cahn, Ingold and Prelog—is S in the 3 position and R in the 3a position.

Moreover, in this connection, compounds of formula I in more particular worthy to be noted are those which have, with respect to the positions 3 and 3a, the same configuration as shown in formula I**:

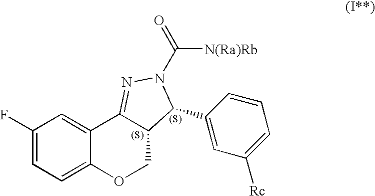

If, for example, in compounds of formula I** Ra, Rb and Rc have the meanings given above, then the configuration—according to the rules of Cahn, Ingold and Prelog—is S in the 3 position and S in the 3a position.

Thus, the enantiomers (3R,3aS) having the formula I* and the salts thereof are a part of the invention, which is in particular to be emphasized.

Further thus, the enantiomers (3S,3aS) having the formula I** and the salts thereof are a part of the invention, which is in more particular to be emphasized.

The invention thus relates to compounds of formula I according to the invention, which have with respect to the positions 3 and 3a the same absolute configuration either as the compound (−)-cis-8-fluoro-3-(3-hydroxy-phenyl)-3a,4-dihydro-3H-chromeno[4,3-c]pyrazole-2-carboxylic acid dimethylamide or as the compound (−)-cis-8-fluoro-3-(3-hydroxy-phenyl)-3a,4-dihydro-3H-chromeno[4,3-c]pyrazole-2-carboxylic acid methyl-(1-methyl-piperidin-4-yl)-amide hydrochloride, as well as the salts thereof.

In one embodiment, preference is given in this connection to those compounds of formula I which have with respect to the positions 3 and 3a the same absolute configuration as the compound (−)-cis-8-fluoro-3-(3-hydroxy-phenyl)-3a,4-dihydro-3H-chromeno[4,3-c]pyrazole-2-carboxylic acid dimethylamide having a specific optical rotation [α]_{D}²⁰ with negative sign (CHCl₃), as well as the salts thereof.

In another embodiment, preference is given in this connection to those compounds of formula I which have with respect to the positions 3 and 3a the same absolute configuration as the compound (−)-cis-8-fluoro-3-(3-hydroxy-phenyl)-3a,4-dihydro-3H-chromeno[4,3-c]pyrazole-2-carboxylic acid methyl-(1-methyl-piperidin-4-yl)-amide hydrochloride having a specific optical rotation [α]_{D}²⁰ with negative sign (c=0.0640, CHCl₃), as well as the salts thereof.

In general, enantiomerically pure compounds of this invention may be prepared according to art-known processes, such as e.g. via asymmetric syntheses, for example by preparation and separation of appropriate diastereoisomeric compounds/intermediates, which can be separated by known methods (e.g. by chromatographic separation or (fractional) crystallization from a suitable solvent), or by using chiral synthons or chiral reagents; by chromatographic separation of the corresponding racemic compounds on chiral separating columns; by means of diastereomeric salt formation of the racemic compounds with optically active acids (such as e.g. those mentioned below) or bases, subsequent resolution of the salts and release of the desired compound from the salt; by derivatization of the racemic compounds with chiral auxiliary reagents, subsequent diastereomer separation and removal of the chiral auxiliary group; by resolution via diastereomeric inclusion compounds (e.g. complexes or clathrates); by kinetic resolution of a racemate (e.g. by enzymatic resolution); by enantioselective (preferential) crystallization (or crystallization by entrainment) from a conglomerate of enantiomorphous crystals under suitable conditions; or by (fractional) crystallization from a suitable solvent in the presence of a chiral auxiliary.

Thus, e.g. one possible alternative for enantiomer separation may be carried out at the stage of the compounds of formula I or of the starting compounds having a protonatable group. Hereby, separation of the enantiomers may be carried out, for example, by means of salt formation of the racemic compounds with optically active acids, especially carboxylic acids, subsequent resolution of the salts and release of the desired compound from the salt. Examples of optically active acids which may be mentioned in this connection, without being restricted thereto, are the enantiomeric forms of mandelic acid, tartaric acid, O,O′-dibenzoyltartaric acid, camphoric acid, quinic acid, glutamic acid, pyroglutamic acid, malic acid, camphorsulfonic acid, 3-bromocamphorsulfonic acid, α-methoxyphenylacetic acid, α-methoxy-α-trifluoromethylphenylacetic acid or 2-phenylpropionic acid or the like.

Another possible alternative for enantiomer separation may be carried out by chromatographic separation of a racemic mixture of compounds of formula I or of starting compounds thereof (e.g. carbamoylchlorides of formula II, in which L is chlorine) on a chiral separating column, such as e.g. described in the following examples or analogously or similarly thereto, using the appropriate separation conditions.

As illustrative compounds according to this invention the following compounds of formula I*,

in which Rc is hydrogen,

and the salts as well as the stereoisomers and salts of the stereoisomers thereof,

may be mentioned by means of the substituent meanings 1) to 139) for —N(Ra)Rb indicated in Table 1 given below.

As further illustrative compounds according to this invention the following compounds of formula I*,

in which Rc is hydroxyl,

and the salts as well as the stereoisomers and salts of the stereoisomers thereof,

may be mentioned by means of the substituent meanings 1) to 139) for —N(Ra)Rb indicated in Table 1 given below.

As further illustrative compounds according to this invention the following compounds of formula I**,

in which Rc is hydrogen,

and the salts as well as the stereoisomers and salts of the stereoisomers thereof,

may be mentioned by means of the substituent meanings 1) to 139) for —N(Ra)Rb indicated in Table 1 given below.

As further illustrative compounds according to this invention the following compounds of formula I**,

in which Rc is hydroxyl,

and the salts as well as the stereoisomers and salts of the stereoisomers thereof,

may be mentioned by means of the substituent meanings 1) to 139) for —N(Ra)Rb indicated in Table 1 given below.

TABLE 1

| | —N(Ra)Rb |
|---|---|
| 1) | 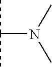 |
| 2) | 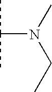 |
| 3) | 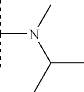 |
| 4) | 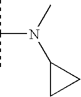 |
| 5) | 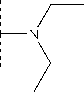 |
| 6) | 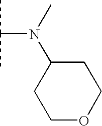 |
| 7) | 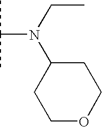 |
| 8) | 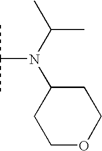 |
| 9) | 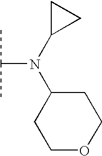 |
| 10) | 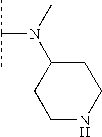 |
| 11) | 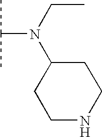 |
| 12) | 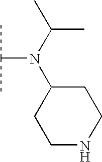 |
| 13) | 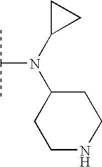 |
| 14) | 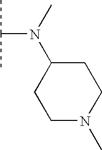 |
| 15) | 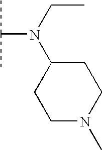 |
| 16) | 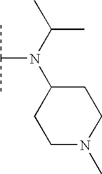 |
| 17) | 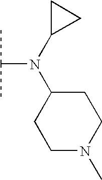 |
| 18) | 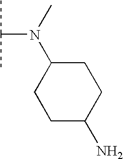 |
| 19) | 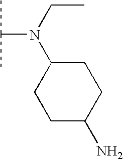 |
| 20) | 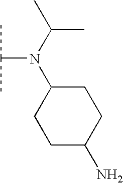 |
| 21) | 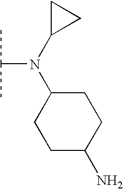 |
| 22) | 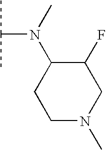 |
| 23) | 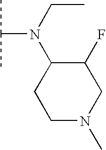 |
| 24) | 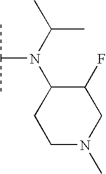 |
| 25) | 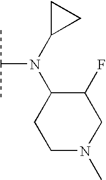 |
| 26) | 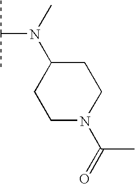 |
| 27) | 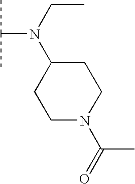 |
| 28) | 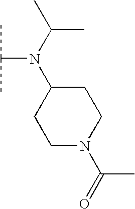 |
| 29) | 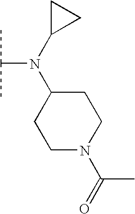 |
| 30) | 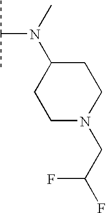 |
| 31) | 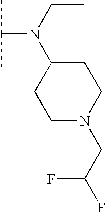 |
| 32) | 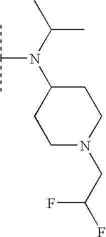 |
| 33) | 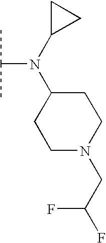 |
| 34) | 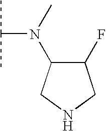 |
| 35) | 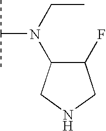 |
| 36) | 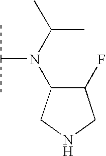 |
| 37) | 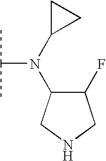 |
| 38) | 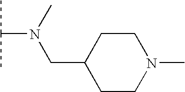 |
| 39) | 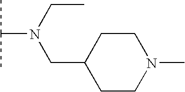 |
| 40) | 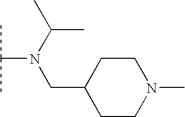 |
| 41) | 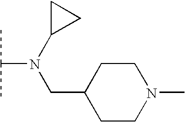 |
| 42) | 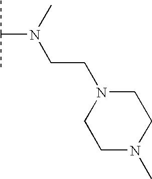 |
| 43) | 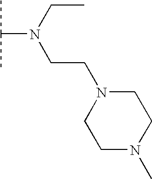 |
| 44) | 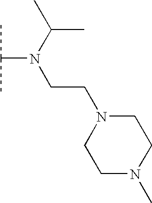 |
| 45) | 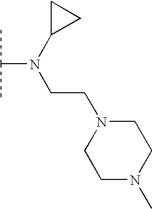 |
| 46) | 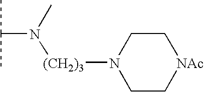 |
| 47) | 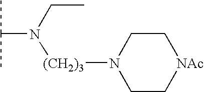 |
| 48) | 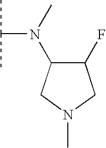 |
| 49) | 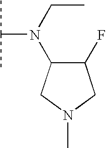 |
| 50) | 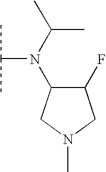 |
| 51) | 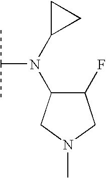 |
| 52) | 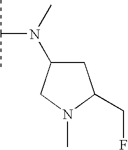 |
| 53) | 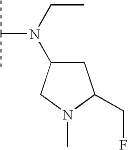 |
| 54) | 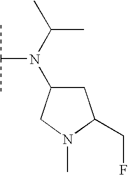 |
| 55) | 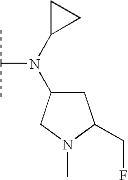 |
| 56) | 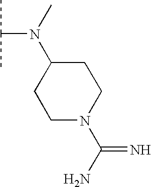 |
| 57) | 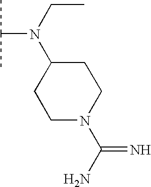 |
| 58) | 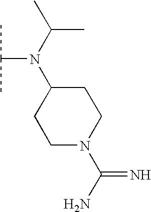 |
| 59) | 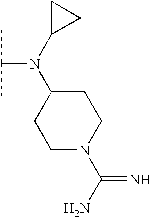 |
| 60) | 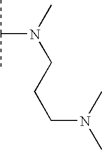 |
| 61) | 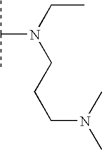 |
| 62) | 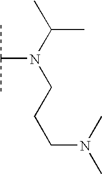 |
| 63) | 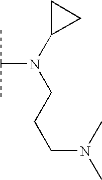 |
| 64) | 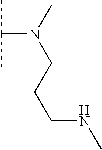 |
| 65) | 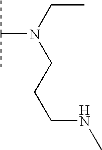 |
| 66) | 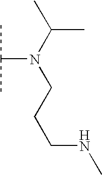 |
| 67) | 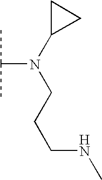 |
| 68) | 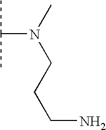 |
| 69) | 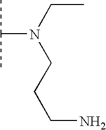 |
| 70) | 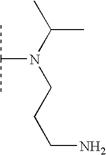 |
| 71) | 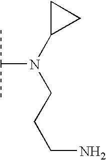 |
| 72) | 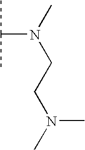 |
| 73) | 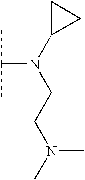 |
| 74) | 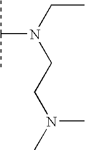 |
| 75) | 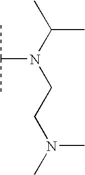 |
| 76) | 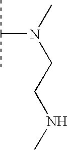 |
| 77) | 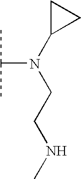 |
| 78) | 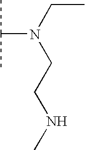 |
| 79) | 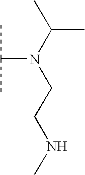 |
| 80) | 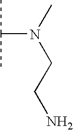 |
| 81) | 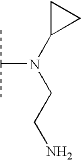 |
| 82) | 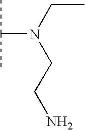 |
| 83) | 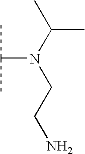 |
| 84) | 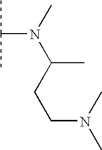 |
| 85) | 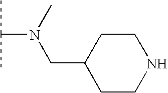 |
| 86) | 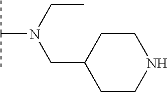 |
| 87) | 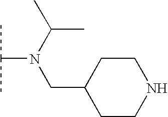 |
| 88) | 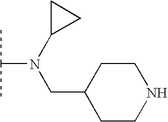 |
| 89) | 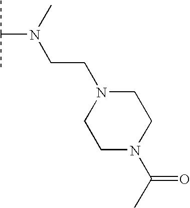 |
| 90) | 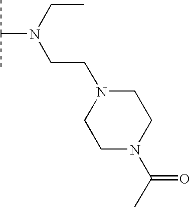 |
| 91) | 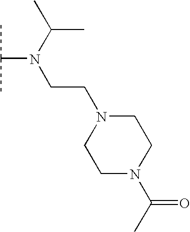 |
| 92) | 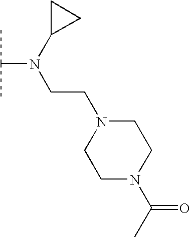 |
| 93) | 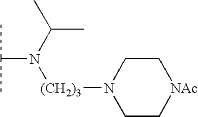 |
| 94) | 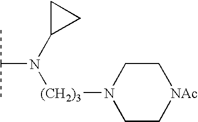 |
| 95) | 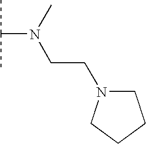 |
| 96) | 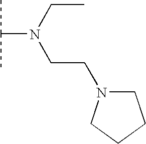 |
| 97) | 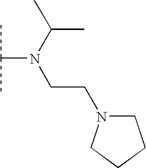 |
| 98) | 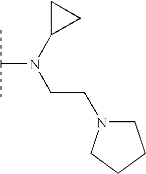 |
| 99) | 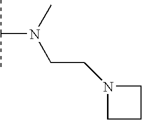 |
| 100) | 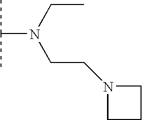 |
| 101) | 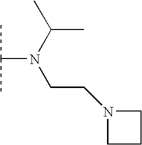 |
| 102) | 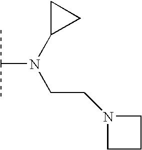 |
| 103) | 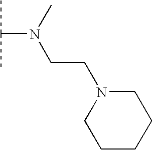 |
| 104) | 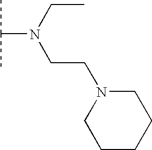 |
| 105) | 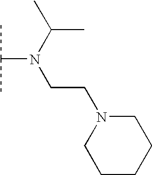 |
| 106) | 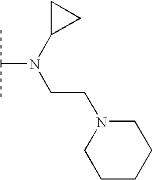 |
| 107) | 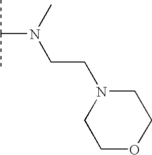 |
| 108) | 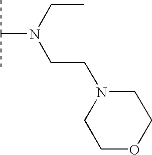 |
| 109) | 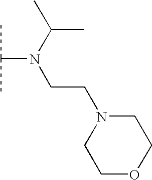 |
| 110) | 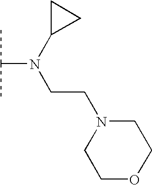 |
| 111) | 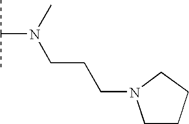 |
| 112) | 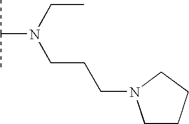 |
| 113) | 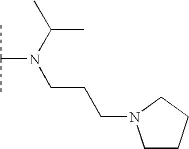 |
| 114) | 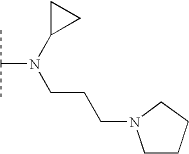 |
| 115) | 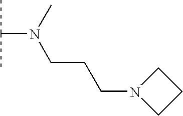 |
| 116) | 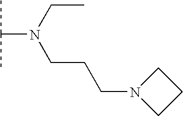 |
| 117) | 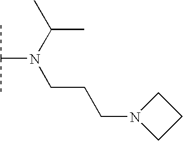 |
| 118) | 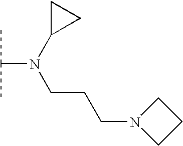 |
| 119) | 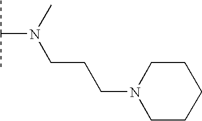 |
| 120) | 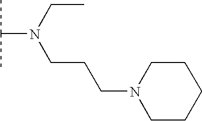 |
| 121) | 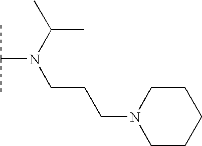 |
| 122) | 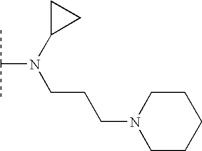 |
| 123) | 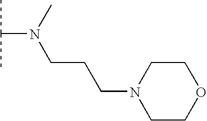 |
| 124) | 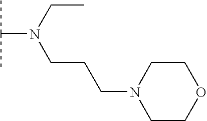 |
| 125) | 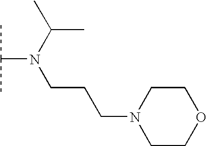 |
| 126) | 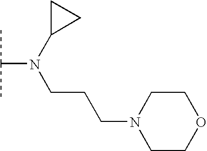 |
| 127) | 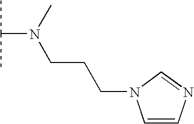 |
| 128) | 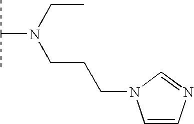 |
| 129) | 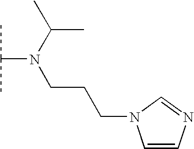 |
| 130) | 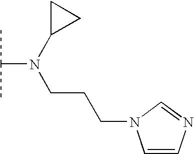 |
| 131) | 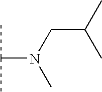 |
| 132) | 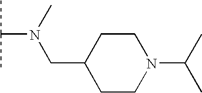 |
| 133) | 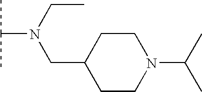 |
| 134) | 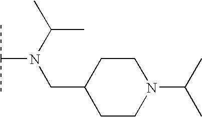 |
| 135) | 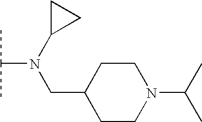 |
| 136) | 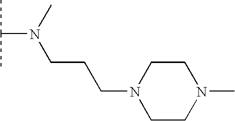 |
| 137) | 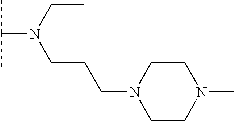 |
| 138) | 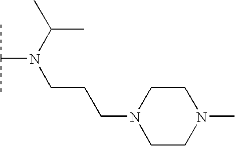 |
| 139) | 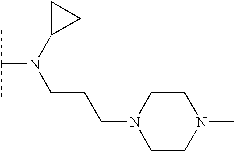 |

Exemplary compounds according to the present invention may include, without being restricted thereto, any compound selected from

(3S,3aR)-8-Fluoro-3-(3-hydroxy-phenyl)-3a,4-dihydro-3H-chromeno[4,3-c]pyrazole-2-carboxylic acid dimethylamide

(3S,3aS)-8-Fluoro-3-(3-hydroxy-phenyl)-3a,4-dihydro-3H-chromeno[4,3-c]pyrazole-2-carboxylic acid dimethylamide

(3S,3aR)-8-Fluoro-3-(3-hydroxy-phenyl)-3a,4-dihydro-3H-chromeno[4,3-c]pyrazole-2-carboxylic acid methyl-(1-methyl-piperidin-4-yl)-amide

(3S,3aS)-8-Fluoro-3-(3-hydroxy-phenyl)-3a,4-dihydro-3H-chromeno[4,3-c]pyrazole-2-carboxylic acid methyl-(1-methyl-piperidin-4-yl)-amide

(3S,3aR)-8-Fluoro-3-(3-hydroxy-phenyl)-3a,4-dihydro-3H-chromeno[4,3-c]pyrazole-2-carboxylic acid (2-dimethylamino-ethyl)-methyl-amide

(3S,3aS)-8-Fluoro-3-(3-hydroxy-phenyl)-3a,4-dihydro-3H-chromeno[4,3-c]pyrazole-2-carboxylic acid (2-dimethylamino-ethyl)-methyl-amide

(3S,3aR)-8-Fluoro-3-(3-hydroxy-phenyl)-3a,4-dihydro-3H-chromeno[4,3-c]pyrazole-2-carboxylic acid cyclopropyl-(1-methyl-piperidin-4-yl)-amide

(3S,3aS)-8-Fluoro-3-(3-hydroxy-phenyl)-3a,4-dihydro-3H-chromeno[4,3-c]pyrazole-2-carboxylic acid cyclopropyl-(1-methyl-piperidin-4-yl)-amide

(3S,3aR)-8-Fluoro-3-(3-hydroxy-phenyl)-3a,4-dihydro-3H-chromeno[4,3-c]pyrazole-2-carboxylic acid (3-dimethylamino-propyl)-methyl-amide

(3S,3aS)-8-Fluoro-3-(3-hydroxy-phenyl)-3a,4-dihydro-3H-chromeno[4,3-c]pyrazole-2-carboxylic acid (3-dimethylamino-propyl)-methyl-amide

(3S,3aR)-8-Fluoro-3-phenyl-3a,4-dihydro-3H-chromeno[4,3-c]pyrazole-2-carboxylic acid dimethylamide

(3S,3aS)-8-Fluoro-3-phenyl-3a,4-dihydro-3H-chromeno[4,3-c]pyrazole-2-carboxylic acid dimethylamide

(3S,3aR)-8-Fluoro-3-phenyl-3a,4-dihydro-3H-chromeno[4,3-c]pyrazole-2-carboxylic acid methyl-(1-methyl-piperidin-4-yl)-amide

(3S,3aS)-8-Fluoro-3-phenyl-3a,4-dihydro-3H-chromeno[4,3-c]pyrazole-2-carboxylic acid methyl-(1-methyl-piperidin-4-yl)-amide

(3S,3aR)-8-Fluoro-3-phenyl-3a,4-dihydro-3H-chromeno[4,3-c]pyrazole-2-carboxylic acid (2-dimethylamino-ethyl)-ethyl-amide

(3S,3aS)-8-Fluoro-3-phenyl-3a,4-dihydro-3H-chromeno[4,3-c]pyrazole-2-carboxylic acid (2-dimethylamino-ethyl)-ethyl-amide

(3S,3aR)-8-Fluoro-3-phenyl-3a,4-dihydro-3H-chromeno[4,3-c]pyrazole-2-carboxylic acid (3-dimethylamino-propyl)-methyl-amide

(3S,3aS)-8-Fluoro-3-phenyl-3a,4-dihydro-3H-chromeno[4,3-c]pyrazole-2-carboxylic acid (3-dimethylamino-propyl)-methyl-amide

(3S,3aR)-8-Fluoro-3-phenyl-3a,4-dihydro-3H-chromeno[4,3-c]pyrazole-2-carboxylic acid isobutyl-methyl-amide

(3S,3aS)-8-Fluoro-3-phenyl-3a,4-dihydro-3H-chromeno[4,3-c]pyrazole-2-carboxylic acid isobutyl-methyl-amide

(3S,3aR)-8-Fluoro-3-phenyl-3a,4-dihydro-3H-chromeno[4,3-c]pyrazole-2-carboxylic acid isopropyl-methyl-amide

(3S,3aS)-8-Fluoro-3-phenyl-3a,4-dihydro-3H-chromeno[4,3-c]pyrazole-2-carboxylic acid isopropyl-methyl-amide

(3S,3aR)-8-Fluoro-3-phenyl-3a,4-dihydro-3H-chromeno[4,3-c]pyrazole-2-carboxylic acid ethyl-methyl-amide

(3S,3aS)-8-Fluoro-3-phenyl-3a,4-dihydro-3H-chromeno[4,3-c]pyrazole-2-carboxylic acid ethyl-methyl-amide

(3S,3aR)-8-Fluoro-3-phenyl-1,3a,4,9b-tetrahydro-3H-chromeno[4,3-c]pyrazole-2-carboxylic acid cyclopropyl-(1-methyl-piperidin-4-yl)-amide

(3S,3aS)-8-Fluoro-3-phenyl-1,3a,4,9b-tetrahydro-3H-chromeno[4,3-c]pyrazole-2-carboxylic acid cyclopropyl-(1-methyl-piperidin-4-yl)-amide

(3S,3aR)-8-Fluoro-3-phenyl-3a,4-dihydro-3H-chromeno[4,3-c]pyrazole-2-carboxylic acid methyl-piperidin-4-yl-amide

(3S,3aS)-8-Fluoro-3-phenyl-3a,4-dihydro-3H-chromeno[4,3-c]pyrazole-2-carboxylic acid methyl-piperidin-4-yl-amide

(3S,3aR)-8-Fluoro-3-phenyl-3a,4-dihydro-3H-chromeno[4,3-c]pyrazole-2-carboxylic acid cyclopropyl-piperidin-4-yl-amide

(3S,3aS)-8-Fluoro-3-phenyl-3a,4-dihydro-3H-chromeno[4,3-c]pyrazole-2-carboxylic acid cyclopropyl-piperidin-4-yl-amide

(3S,3aR)-8-Fluoro-3-phenyl-3a,4-dihydro-3H-chromeno[4,3-c]pyrazole-2-carboxylic acid (2-amino-ethyl)-methyl-amide

(3S,3aS)-8-Fluoro-3-phenyl-3a,4-dihydro-3H-chromeno[4,3-c]pyrazole-2-carboxylic acid (2-amino-ethyl)-methyl-amide

(3S,3aR)-8-Fluoro-3-phenyl-3a,4-dihydro-3H-chromeno[4,3-c]pyrazole-2-carboxylic acid (2-dimethylamino-ethyl)-methyl-amide

(3S,3aS)-8-Fluoro-3-phenyl-3a,4-dihydro-3H-chromeno[4,3-c]pyrazole-2-carboxylic acid (2-dimethylamino-ethyl)-methyl-amide

(3S,3aR)-8-Fluoro-3-phenyl-3a,4-dihydro-3H-chromeno[4,3-c]pyrazole-2-carboxylic acid (3-amino-propyl)-methyl-amide

(3S,3aS)-8-Fluoro-3-phenyl-3a,4-dihydro-3H-chromeno[4,3-c]pyrazole-2-carboxylic acid (3-amino-propyl)-methyl-amide

(3S,3aR)-8-Fluoro-3-phenyl-3a,4-dihydro-3H-chromeno[4,3-c]pyrazole-2-carboxylic acid (1-acetyl-piperidin-4-yl)-cyclopropyl-amide

(3S,3aS)-8-Fluoro-3-phenyl-3a,4-dihydro-3H-chromeno[4,3-c]pyrazole-2-carboxylic acid (1-acetyl-piperidin-4-yl)-cyclopropyl-amide

(3S,3aR)-8-Fluoro-3-phenyl-3a,4-dihydro-3H-chromeno[4,3-c]pyrazole-2-carboxylic acid methyl-(3-methylamino-propyl)-amide

(3S,3aS)-8-Fluoro-3-phenyl-3a,4-dihydro-3H-chromeno[4,3-c]pyrazole-2-carboxylic acid methyl-(3-methylamino-propyl)-amide

(3S,3aR)-8-Fluoro-3-phenyl-3a,4-dihydro-3H-chromeno[4,3-c]pyrazole-2-carboxylic acid methyl-(2-methylamino-ethyl)-amide

(3S,3aS)-8-Fluoro-3-phenyl-3a,4-dihydro-3H-chromeno[4,3-c]pyrazole-2-carboxylic acid methyl-(2-methylamino-ethyl)-amide

(3S,3aR)-8-Fluoro-3-phenyl-3a,4-dihydro-3H-chromeno[4,3-c]pyrazole-2-carboxylic acid methyl-((RS)-1-methyl-piperidin-3-ylmethyl)-amide

(3S,3aS)-8-Fluoro-3-phenyl-3a,4-dihydro-3H-chromeno[4,3-c]pyrazole-2-carboxylic acid methyl-((RS)-1-methyl-piperidin-3-ylmethyl)-amide

(3S,3aR)-8-Fluoro-3-phenyl-3a,4-dihydro-3H-chromeno[4,3-c]pyrazole-2-carboxylic acid (RS)-methyl-piperidin-3-yl-amide

(3S,3aS)-8-Fluoro-3-phenyl-3a,4-dihydro-3H-chromeno[4,3-c]pyrazole-2-carboxylic acid (RS)-methyl-piperidin-3-yl-amide

(3S,3aR)-8-Fluoro-3-phenyl-3a,4-dihydro-3H-chromeno[4,3-c]pyrazole-2-carboxylic acid ((RS)-1-isopropyl-piperidin-3-ylmethyl)-methyl-amide

(3S,3aS)-8-Fluoro-3-phenyl-3a,4-dihydro-3H-chromeno[4,3-c]pyrazole-2-carboxylic acid ((RS)-1-isopropyl-piperidin-3-ylmethyl)-methyl-amide

(3S,3aR)-8-Fluoro-3-(3-hydroxy-phenyl)-3a,4-dihydro-3H-chromeno[4,3-c]pyrazole-2-carboxylic acid methyl-piperidin-4-yl-amide

(3S,3aS)-8-Fluoro-3-(3-hydroxy-phenyl)-3a,4-dihydro-3H-chromeno[4,3-c]pyrazole-2-carboxylic acid methyl-piperidin-4-yl-amide

(3S,3aR)-8-Fluoro-3-phenyl-3a,4-dihydro-3H-chromeno[4,3-c]pyrazole-2-carboxylic acid methyl-(2-pyrrolidin-1-yl-ethyl)-amide and

(3S,3aS)-8-Fluoro-3-phenyl-3a,4-dihydro-3H-chromeno[4,3-c]pyrazole-2-carboxylic acid methyl-(2-pyrrolidin-1-yl-ethyl)-amide and

(3S,3aR)-8-Fluoro-3-phenyl-3a,4-dihydro-3H-chromeno[4,3-c]pyrazole-2-carboxylic acid [2-(4-acetyl-piperazin-1-yl)-ethyl]-methyl-amide

(3S,3aS)-8-Fluoro-3-phenyl-3a,4-dihydro-3H-chromeno[4,3-c]pyrazole-2-carboxylic acid [2-(4-acetyl-piperazin-1-yl)-ethyl]-methyl-amide

(3S,3aR)-8-Fluoro-3-(3-hydroxy-phenyl)-3a,4-dihydro-3H-chromeno[4,3-c]pyrazole-2-carboxylic acid (2-dimethylamino-ethyl)-ethyl-amide

(3S,3aS)-8-Fluoro-3-(3-hydroxy-phenyl)-3a,4-dihydro-3H-chromeno[4,3-c]pyrazole-2-carboxylic acid (2-dimethylamino-ethyl)-ethyl-amide

(3S,3aR)-8-Fluoro-3-(3-hydroxy-phenyl)-3a,4-dihydro-3H-chromeno[4,3-c]pyrazole-2-carboxylic acid isobutyl-methyl-amide

(3S,3aS)-8-Fluoro-3-(3-hydroxy-phenyl)-3a,4-dihydro-3H-chromeno[4,3-c]pyrazole-2-carboxylic acid isobutyl-methyl-amide

(3S,3aR)-8-Fluoro-3-(3-hydroxy-phenyl)-3a,4-dihydro-3H-chromeno[4,3-c]pyrazole-2-carboxylic acid isopropyl-methyl-amide

(3S,3aS)-8-Fluoro-3-(3-hydroxy-phenyl)-3a,4-dihydro-3H-chromeno[4,3-c]pyrazole-2-carboxylic acid isopropyl-methyl-amide

(3S,3aR)-8-Fluoro-3-(3-hydroxy-phenyl)-3a,4-dihydro-3H-chromeno[4,3-c]pyrazole-2-carboxylic acid ethyl-methyl-amide

(3S,3aS)-8-Fluoro-3-(3-hydroxy-phenyl)-3a,4-dihydro-3H-chromeno[4,3-c]pyrazole-2-carboxylic acid ethyl-methyl-amide

(3S,3aR)-8-Fluoro-3-(3-hydroxy-phenyl)-3a,4-dihydro-3H-chromeno[4,3-c]pyrazole-2-carboxylic acid cyclopropyl-piperidin-4-yl-amide

(3S,3aS)-8-Fluoro-3-(3-hydroxy-phenyl)-3a,4-dihydro-3H-chromeno[4,3-c]pyrazole-2-carboxylic acid cyclopropyl-piperidin-4-yl-amide

(3S,3aR)-8-Fluoro-3-(3-hydroxy-phenyl)-3a,4-dihydro-3H-chromeno[4,3-c]pyrazole-2-carboxylic acid (2-amino-ethyl)-methyl-amide

(3S,3aS)-8-Fluoro-3-(3-hydroxy-phenyl)-3a,4-dihydro-3H-chromeno[4,3-c]pyrazole-2-carboxylic acid (2-amino-ethyl)-methyl-amide

(3S,3aR)-8-Fluoro-3-(3-hydroxy-phenyl)-3a,4-dihydro-3H-chromeno[4,3-c]pyrazole-2-carboxylic acid (3-amino-propyl)-methyl-amide

(3S,3aS)-8-Fluoro-3-(3-hydroxy-phenyl)-3a,4-dihydro-3H-chromeno[4,3-c]pyrazole-2-carboxylic acid (3-amino-propyl)-methyl-amide

(3S,3aR)-8-Fluoro-3-(3-hydroxy-phenyl)-3a,4-dihydro-3H-chromeno[4,3-c]pyrazole-2-carboxylic acid (1-acetyl-piperidin-4-yl)-cyclopropyl-amide

(3S,3aS)-8-Fluoro-3-(3-hydroxy-phenyl)-3a,4-dihydro-3H-chromeno[4,3-c]pyrazole-2-carboxylic acid (1-acetyl-piperidin-4-yl)-cyclopropyl-amide

(3S,3aR)-8-Fluoro-3-(3-hydroxy-phenyl)-3a,4-dihydro-3H-chromeno[4,3-c]pyrazole-2-carboxylic acid methyl-(3-methylamino-propyl)-amide

(3S,3aS)-8-Fluoro-3-(3-hydroxy-phenyl)-3a,4-dihydro-3H-chromeno[4,3-c]pyrazole-2-carboxylic acid methyl-(3-methylamino-propyl)-amide

(3S,3aR)-8-Fluoro-3-(3-hydroxy-phenyl)-3a,4-dihydro-3H-chromeno[4,3-c]pyrazole-2-carboxylic acid methyl-(2-methylamino-ethyl)-amide

(3S,3aS)-8-Fluoro-3-(3-hydroxy-phenyl)-3a,4-dihydro-3H-chromeno[4,3-c]pyrazole-2-carboxylic acid methyl-(2-methylamino-ethyl)-amide

(3S,3aR)-8-Fluoro-3-(3-hydroxy-phenyl)-3a,4-dihydro-3H-chromeno[4,3-c]pyrazole-2-carboxylic acid methyl-((RS)-1-methyl-piperidin-3-ylmethyl)-amide

(3S,3aS)-8-Fluoro-3-(3-hydroxy-phenyl)-3a,4-dihydro-3H-chromeno[4,3-c]pyrazole-2-carboxylic acid methyl-((RS)-1-methyl-piperidin-3-ylmethyl)-amide

(3S,3aR)-8-Fluoro-3-(3-hydroxy-phenyl)-3a,4-dihydro-3H-chromeno[4,3-c]pyrazole-2-carboxylic acid (RS)-methyl-piperidin-3-yl-amide

(3S,3aS)-8-Fluoro-3-(3-hydroxy-phenyl)-3a,4-dihydro-3H-chromeno[4,3-c]pyrazole-2-carboxylic acid (RS)-methyl-piperidin-3-yl-amide

(3S,3aR)-8-Fluoro-3-(3-hydroxy-phenyl)-3a,4-dihydro-3H-chromeno[4,3-c]pyrazole-2-carboxylic acid methyl-(2-pyrrolidin-1-yl-ethyl)-amide

(3S,3aS)-8-Fluoro-3-(3-hydroxy-phenyl)-3a,4-dihydro-3H-chromeno[4,3-c]pyrazole-2-carboxylic acid methyl-(2-pyrrolidin-1-yl-ethyl)-amide

(3S,3aR)-8-Fluoro-3-(3-hydroxy-phenyl)-3a,4-dihydro-3H-chromeno[4,3-c]pyrazole-2-carboxylic acid [2-(4-acetyl-piperazin-1-yl)-ethyl]-methyl-amide

(3S,3aS)-8-Fluoro-3-(3-hydroxy-phenyl)-3a,4-dihydro-3H-chromeno[4,3-c]pyrazole-2-carboxylic acid [2-(4-acetyl-piperazin-1-yl)-ethyl]-methyl-amide

(3S,3aR)-8-Fluoro-3-phenyl-3a,4-dihydro-3H-chromeno[4,3-c]pyrazole-2-carboxylic acid (3-dimethylamino-propyl)-ethyl-amide

(3S,3aS)-8-Fluoro-3-phenyl-3a,4-dihydro-3H-chromeno[4,3-c]pyrazole-2-carboxylic acid (3-dimethylamino-propyl)-ethyl-amide

(3S,3aR)-8-Fluoro-3-phenyl-3a,4-dihydro-3H-chromeno[4,3-c]pyrazole-2-carboxylic acid methyl-(3-pyrrolidin-1-yl-propyl)-amide

(3S,3aS)-8-Fluoro-3-phenyl-3a,4-dihydro-3H-chromeno[4,3-c]pyrazole-2-carboxylic acid methyl-(3-pyrrolidin-1-yl-propyl)-amide

(3S,3aR)-8-Fluoro-3-phenyl-3a,4-dihydro-3H-chromeno[4,3-c]pyrazole-2-carboxylic acid [3-(4-acetyl-piperazin-1-yl)-propyl]-methyl-amide

(3S,3aS)-8-Fluoro-3-phenyl-3a,4-dihydro-3H-chromeno[4,3-c]pyrazole-2-carboxylic acid [3-(4-acetyl-piperazin-1-yl)-propyl]-methyl-amide

(3S,3aR)-8-Fluoro-3-(3-hydroxy-phenyl)-3a,4-dihydro-3H-chromeno[4,3-c]pyrazole-2-carboxylic acid (3-dimethylamino-propyl)-ethyl-amide

(3S,3aS)-8-Fluoro-3-(3-hydroxy-phenyl)-3a,4-dihydro-3H-chromeno[4,3-c]pyrazole-2-carboxylic acid (3-dimethylamino-propyl)-ethyl-amide

(3S,3aR)-8-Fluoro-3-(3-hydroxy-phenyl)-3a,4-dihydro-3H-chromeno[4,3-c]pyrazole-2-carboxylic acid methyl-(3-pyrrolidin-1-yl-propyl)-amide

(3S,3aS)-8-Fluoro-3-(3-hydroxy-phenyl)-3a,4-dihydro-3H-chromeno[4,3-c]pyrazole-2-carboxylic acid methyl-(3-pyrrolidin-1-yl-propyl)-amide

(3S,3aR)-8-Fluoro-3-(3-hydroxy-phenyl)-3a,4-dihydro-3H-chromeno[4,3-c]pyrazole-2-carboxylic acid [3-(4-acetyl-piperazin-1-yl)-propyl]-methyl-amide and

(3S,3aS)-8-Fluoro-3-(3-hydroxy-phenyl)-3a,4-dihydro-3H-chromeno[4,3-c]pyrazole-2-carboxylic acid [3-(4-acetyl-piperazin-1-yl)-propyl]-methyl-amide

and the salts as well as the stereoisomers and salts of the stereoisomers thereof.

Particular exemplary compounds according to the present invention may include, without being restricted thereto, any compound selected from

1. (3S,3aR)-8-Fluoro-3-(3-hydroxy-phenyl)-3a,4-dihydro-3H-chromeno[4,3-c]pyrazole-2-carboxylic acid dimethylamide

2. (3S,3aS)-8-Fluoro-3-(3-hydroxy-phenyl)-3a,4-dihydro-3H-chromeno[4,3-c]pyrazole-2-carboxylic acid dimethylamide

3. (3S,3aS)-8-Fluoro-3-(3-hydroxy-phenyl)-3a,4-dihydro-3H-chromeno[4,3-c]pyrazole-2-carboxylic acid methyl-(1-methyl-piperidin-4-yl)-amide

4. (3S,3aS)-8-Fluoro-3-(3-hydroxy-phenyl)-3a,4-dihydro-3H-chromeno[4,3-c]pyrazole-2-carboxylic acid (2-dimethylamino-ethyl)-methyl-amide

5. (3S,3aS)-8-Fluoro-3-(3-hydroxy-phenyl)-3a,4-dihydro-3H-chromeno[4,3-c]pyrazole-2-carboxylic acid cyclopropyl-(1-methyl-piperidin-4-yl)-amide

6. (3S,3aS)-8-Fluoro-3-(3-hydroxy-phenyl)-3a,4-dihydro-3H-chromeno[4,3-c]pyrazole-2-carboxylic acid (3-dimethylamino-propyl)-methyl-amide

7. (3S,3aS)-8-Fluoro-3-phenyl-3a,4-dihydro-3H-chromeno[4,3-c]pyrazole-2-carboxylic acid dimethylamide

8. (3S,3aR)-8-Fluoro-3-phenyl-3a,4-dihydro-3H-chromeno[4,3-c]pyrazole-2-carboxylic acid dimethylamide

9. (3S,3aS)-8-Fluoro-3-phenyl-3a,4-dihydro-3H-chromeno[4,3-c]pyrazole-2-carboxylic acid methyl-(1-methyl-piperidin-4-yl)-amide

10. (3S,3aS)-8-Fluoro-3-phenyl-3a,4-dihydro-3H-chromeno[4,3-c]pyrazole-2-carboxylic acid (2-dimethylamino-ethyl)-ethyl-amide

11. (3S,3aS)-8-Fluoro-3-phenyl-3a,4-dihydro-3H-chromeno[4,3-c]pyrazole-2-carboxylic acid (3-dimethylamino-propyl)-methyl-amide

12. (3S,3aS)-8-Fluoro-3-phenyl-3a,4-dihydro-3H-chromeno[4,3-c]pyrazole-2-carboxylic acid isobutyl-methyl-amide

13. (3S,3aS)-8-Fluoro-3-phenyl-3a,4-dihydro-3H-chromeno[4,3-c]pyrazole-2-carboxylic acid isopropyl-methyl-amide

14. (3S,3aS)-8-Fluoro-3-phenyl-3a,4-dihydro-3H-chromeno[4,3-c]pyrazole-2-carboxylic acid ethyl-methyl-amide

15. (3S,3aS)-8-Fluoro-3-phenyl-1,3a,4,9b-tetrahydro-3H-chromeno[4,3-c]pyrazole-2-carboxylic acid cyclopropyl-(1-methyl-piperidin-4-yl)-amide

16. (3S,3aS)-8-Fluoro-3-phenyl-3a,4-dihydro-3H-chromeno[4,3-c]pyrazole-2-carboxylic acid methyl-piperidin-4-yl-amide

17. (3S,3aS)-8-Fluoro-3-phenyl-3a,4-dihydro-3H-chromeno[4,3-c]pyrazole-2-carboxylic acid cyclopropyl-piperidin-4-yl-amide

18. (3S,3aS)-8-Fluoro-3-phenyl-3a,4-dihydro-3H-chromeno[4,3-c]pyrazole-2-carboxylic acid (2-amino-ethyl)-methyl-amide

19. (3S,3aS)-8-Fluoro-3-phenyl-3a,4-dihydro-3H-chromeno[4,3-c]pyrazole-2-carboxylic acid (2-dimethylamino-ethyl)-methyl-amide

20. (3S,3aS)-8-Fluoro-3-phenyl-3a,4-dihydro-3H-chromeno[4,3-c]pyrazole-2-carboxylic acid (3-amino-propyl)-methyl-amide

21. (3S,3aS)-8-Fluoro-3-phenyl-3a,4-dihydro-3H-chromeno[4,3-c]pyrazole-2-carboxylic acid (1-acetyl-piperidin-4-yl)-cyclopropyl-amide

22. (3S,3aS)-8-Fluoro-3-phenyl-3a,4-dihydro-3H-chromeno[4,3-c]pyrazole-2-carboxylic acid methyl-(3-methylamino-propyl)-amide

23. (3S,3aS)-8-Fluoro-3-phenyl-3a,4-dihydro-3H-chromeno[4,3-c]pyrazole-2-carboxylic acid methyl-(2-methylamino-ethyl)-amide

24. (3S,3aS)-8-Fluoro-3-phenyl-3a,4-dihydro-3H-chromeno[4,3-c]pyrazole-2-carboxylic acid methyl-((RS)-1-methyl-piperidin-3-ylmethyl)-amide

25. (3S,3aS)-8-Fluoro-3-phenyl-3a,4-dihydro-3H-chromeno[4,3-c]pyrazole-2-carboxylic acid (RS)-methyl-piperidin-3-yl-amide

26. (3S,3aS)-8-Fluoro-3-phenyl-3a,4-dihydro-3H-chromeno[4,3-c]pyrazole-2-carboxylic acid ((RS)-1-isopropyl-piperidin-3-ylmethyl)-methyl-amide

27. (3S,3aS)-8-Fluoro-3-(3-hydroxy-phenyl)-3a,4-dihydro-3H-chromeno[4,3-c]pyrazole-2-carboxylic acid methyl-piperidin-4-yl-amide

28. (3S,3aS)-8-Fluoro-3-phenyl-3a,4-dihydro-3H-chromeno[4,3-c]pyrazole-2-carboxylic acid methyl-(2-pyrrolidin-1-yl-ethyl)-amide and

29. (3S,3aS)-8-Fluoro-3-phenyl-3a,4-dihydro-3H-chromeno[4,3-c]pyrazole-2-carboxylic acid [2-(4-acetyl-piperazin-1-yl)-ethyl]-methyl-amide

and the salts as well as the stereoisomers and salts of the stereoisomers thereof.

In a preferred embodiment, the invention relates to a compound according to the invention, which is selected from

(1) (3RS,3aSR)-8-Fluoro-3-(3-hydroxy-phenyl)-3a,4-dihydro-3H-chromeno[4,3-c]pyrazole-2-carboxylic acid dimethylamide,

(2) (3RS,3aRS)-8-Fluoro-3-(3-hydroxy-phenyl)-3a,4-dihydro-3H-chromeno[4,3-c]pyrazole-2-carboxylic acid dimethylamide,

(3) (3RS,3aRS)-8-Fluoro-3-(3-hydroxy-phenyl)-3a,4-dihydro-3H-chromeno[4,3-c]pyrazole-2-carboxylic acid methyl-(1-methyl-piperidin-4-yl)-amide,

(4) (3RS,3aRS)-8-Fluoro-3-(3-hydroxy-phenyl)-3a,4-dihydro-3H-chromeno[4,3-c]pyrazole-2-carboxylic acid (2-dimethylamino-ethyl)-methyl-amide,

(5) (3RS,3aRS)-8-Fluoro-3-(3-hydroxy-phenyl)-3a,4-dihydro-3H-chromeno[4,3-c]pyrazole-2-carboxylic acid cyclopropyl-(1-methyl-piperidin-4-yl)-amide,

(6) (3RS,3aRS)-8-Fluoro-3-(3-hydroxy-phenyl)-3a,4-dihydro-3H-chromeno[4,3-c]pyrazole-2-carboxylic acid (3-dimethylamino-propyl)-methyl-amide,

(7) (3RS,3aRS)-8-Fluoro-3-phenyl-3a,4-dihydro-3H-chromeno[4,3-c]pyrazole-2-carboxylic acid dimethylamide,

(8) (3RS,3aSR)-8-Fluoro-3-phenyl-3a,4-dihydro-3H-chromeno[4,3-c]pyrazole-2-carboxylic acid dimethylamide,

(9) (3RS,3aRS)-8-Fluoro-3-phenyl-3a,4-dihydro-3H-chromeno[4,3-c]pyrazole-2-carboxylic acid methyl-(1-methyl-piperidin-4-yl)-amide,

(10) (3RS,3aRS)-8-Fluoro-3-phenyl-3a,4-dihydro-3H-chromeno[4,3-c]pyrazole-2-carboxylic acid (2-dimethylamino-ethyl)-ethyl-amide,

(11) (3RS,3aRS)-8-Fluoro-3-phenyl-3a,4-dihydro-3H-chromeno[4,3-c]pyrazole-2-carboxylic acid (3-dimethylamino-propyl)-methyl-amide,

(12) (3RS,3aRS)-8-Fluoro-3-phenyl-3a,4-dihydro-3H-chromeno[4,3-c]pyrazole-2-carboxylic acid isobutyl-methyl-amide,

(13) (3RS,3aRS)-8-Fluoro-3-phenyl-3a,4-dihydro-3H-chromeno[4,3-c]pyrazole-2-carboxylic acid isopropyl-methyl-amide,

(14) (3RS,3aRS)-8-Fluoro-3-phenyl-3a,4-dihydro-3H-chromeno[4,3-c]pyrazole-2-carboxylic acid ethyl-methyl-amide,

(15) (3RS,3aRS)-8-Fluoro-3-phenyl-1,3a,4,9b-tetrahydro-3H-chromeno[4,3-c]pyrazole-2-carboxylic acid cyclopropyl-(1-methyl-piperidin-4-yl)-amide,

(16) (3RS,3aRS)-8-Fluoro-3-phenyl-3a,4-dihydro-3H-chromeno[4,3-c]pyrazole-2-carboxylic acid methyl-piperidin-4-yl-amide,

(17) (3RS,3aRS)-8-Fluoro-3-phenyl-3a,4-dihydro-3H-chromeno[4,3-c]pyrazole-2-carboxylic acid cyclopropyl-piperidin-4-yl-amide,

(18) (3RS,3aRS)-8-Fluoro-3-phenyl-3a,4-dihydro-3H-chromeno[4,3-c]pyrazole-2-carboxylic acid (2-amino-ethyl)-methyl-amide,

(19) (3RS,3aRS)-8-Fluoro-3-phenyl-3a,4-dihydro-3H-chromeno[4,3-c]pyrazole-2-carboxylic acid (2-dimethylamino-ethyl)-methyl-amide,

(20) (3RS,3aRS)-8-Fluoro-3-phenyl-3a,4-dihydro-3H-chromeno[4,3-c]pyrazole-2-carboxylic acid (3-amino-propyl)-methyl-amide,

(21) (3RS,3aRS)-8-Fluoro-3-phenyl-3a,4-dihydro-3H-chromeno[4,3-c]pyrazole-2-carboxylic acid (1-acetyl-piperidin-4-yl)-cyclopropyl-amide,

(22) (3RS,3aRS)-8-Fluoro-3-phenyl-3a,4-dihydro-3H-chromeno[4,3-c]pyrazole-2-carboxylic acid methyl-(3-methylamino-propyl)-amide,

(23) (3RS,3aRS)-8-Fluoro-3-phenyl-3a,4-dihydro-3H-chromeno[4,3-c]pyrazole-2-carboxylic acid methyl-(2-methylamino-ethyl)-amide,

(24) (3RS,3aRS)-8-Fluoro-3-phenyl-3a,4-dihydro-3H-chromeno[4,3-c]pyrazole-2-carboxylic acid methyl-(1-methyl-piperidin-3-ylmethyl)-amide with (R)— or (S)-methyl-piperidin-3-yl or mixtures thereof,

(25) (3RS,3aRS)-8-Fluoro-3-phenyl-3a,4-dihydro-3H-chromeno[4,3-c]pyrazole-2-carboxylic acid-methyl-piperidin-3-yl-amide with (R)— or (S)-methyl-piperidin-3-yl or mixtures thereof,

(26) (3RS,3aRS)-8-Fluoro-3-phenyl-3a,4-dihydro-3H-chromeno[4,3-c]pyrazole-2-carboxylic acid (-1-isopropyl-piperidin-3-ylmethyl)-methyl-amide with (R)— or (S)-1-isopropyl-piperidin-3-yl or mixtures thereof,

(27) (3RS,3aRS)-8-Fluoro-3-(3-hydroxy-phenyl)-3a,4-dihydro-3H-chromeno[4,3-c]pyrazole-2-carboxylic acid methyl-piperidin-4-yl-amide,

(28) (3RS,3aRS)-8-Fluoro-3-phenyl-3a,4-dihydro-3H-chromeno[4,3-c]pyrazole-2-carboxylic acid methyl-(2-pyrrolidin-1-yl-ethyl)-amide,

(29) ((3RS,3aRS)-8-Fluoro-3-phenyl-3a,4-dihydro-3H-chromeno[4,3-c]pyrazole-2-carboxylic acid [2-(4-acetyl-piperazin-1-yl)-ethyl]-methyl-amide,

or a salt, stereoisomer or salt of a stereoisomer thereof.

In a preferred embodiment, the invention relates to a compound according to the invention, which is selected from

(1) (3RS,3aSR)-8-Fluoro-3-(3-hydroxy-phenyl)-3a,4-dihydro-3H-chromeno[4,3-c]pyrazole-2-carboxylic acid dimethylamide,

(2) (3RS,3aRS)-8-Fluoro-3-(3-hydroxy-phenyl)-3a,4-dihydro-3H-chromeno[4,3-c]pyrazole-2-carboxylic acid dimethylamide,

(3) (3RS,3aRS)-8-Fluoro-3-(3-hydroxy-phenyl)-3a,4-dihydro-3H-chromeno[4,3-c]pyrazole-2-carboxylic acid methyl-(1-methyl-piperidin-4-yl)-amide,

(4) (3RS,3aRS)-8-Fluoro-3-(3-hydroxy-phenyl)-3a,4-dihydro-3H-chromeno[4,3-c]pyrazole-2-carboxylic acid (2-dimethylamino-ethyl)-methyl-amide,

(5) (3RS,3aRS)-8-Fluoro-3-(3-hydroxy-phenyl)-3a,4-dihydro-3H-chromeno[4,3-c]pyrazole-2-carboxylic acid cyclopropyl-(1-methyl-piperidin-4-yl)-amide,

(6) (3RS,3aRS)-8-Fluoro-3-(3-hydroxy-phenyl)-3a,4-dihydro-3H-chromeno[4,3-c]pyrazole-2-carboxylic acid (3-dimethylamino-propyl)-methyl-amide,

(7) (3RS,3aRS)-8-Fluoro-3-phenyl-3a,4-dihydro-3H-chromeno[4,3-c]pyrazole-2-carboxylic acid dimethylamide,

(8) (3RS,3aSR)-8-Fluoro-3-phenyl-3a,4-dihydro-3H-chromeno[4,3-c]pyrazole-2-carboxylic acid dimethylamide,

(9) (3RS,3aRS)-8-Fluoro-3-phenyl-3a,4-dihydro-3H-chromeno[4,3-c]pyrazole-2-carboxylic acid methyl-(1-methyl-piperidin-4-yl)-amide,

(10) (3RS,3aRS)-8-Fluoro-3-phenyl-3a,4-dihydro-3H-chromeno[4,3-c]pyrazole-2-carboxylic acid (2-dimethylamino-ethyl)-ethyl-amide,

(11) (3RS,3aRS)-8-Fluoro-3-phenyl-3a,4-dihydro-3H-chromeno[4,3-c]pyrazole-2-carboxylic acid (3-dimethylamino-propyl)-methyl-amide,

(12) (3RS,3aRS)-8-Fluoro-3-phenyl-3a,4-dihydro-3H-chromeno[4,3-c]pyrazole-2-carboxylic acid isobutyl-methyl-amide,

(13) (3RS,3aRS)-8-Fluoro-3-phenyl-3a,4-dihydro-3H-chromeno[4,3-c]pyrazole-2-carboxylic acid isopropyl-methyl-amide,

(14) (3RS,3aRS)-8-Fluoro-3-phenyl-3a,4-dihydro-3H-chromeno[4,3-c]pyrazole-2-carboxylic acid ethyl-methyl-amide,

(15) (3RS,3aRS)-8-Fluoro-3-phenyl-1,3a,4,9b-tetrahydro-3H-chromeno[4,3-c]pyrazole-2-carboxylic acid cyclopropyl-(1-methyl-piperidin-4-yl)-amide,

(16) (3RS,3aRS)-8-Fluoro-3-phenyl-3a,4-dihydro-3H-chromeno[4,3-c]pyrazole-2-carboxylic acid methyl-piperidin-4-yl-amide,

(17) (3RS,3aRS)-8-Fluoro-3-phenyl-3a,4-dihydro-3H-chromeno[4,3-c]pyrazole-2-carboxylic acid cyclopropyl-piperidin-4-yl-amide,

(18) (3RS,3aRS)-8-Fluoro-3-phenyl-3a,4-dihydro-3H-chromeno[4,3-c]pyrazole-2-carboxylic acid (2-amino-ethyl)-methyl-amide,

(19) (3RS,3aRS)-8-Fluoro-3-phenyl-3a,4-dihydro-3H-chromeno[4,3-c]pyrazole-2-carboxylic acid (2-dimethylamino-ethyl)-methyl-amide,

(20) (3RS,3aRS)-8-Fluoro-3-phenyl-3a,4-dihydro-3H-chromeno[4,3-c]pyrazole-2-carboxylic acid (3-amino-propyl)-methyl-amide,

(21) (3RS,3aRS)-8-Fluoro-3-phenyl-3a,4-dihydro-3H-chromeno[4,3-c]pyrazole-2-carboxylic acid (1-acetyl-piperidin-4-yl)-cyclopropyl-amide,

(22) (3RS,3aRS)-8-Fluoro-3-phenyl-3a,4-dihydro-3H-chromeno[4,3-c]pyrazole-2-carboxylic acid methyl-(3-methylamino-propyl)-amide,

(23) (3RS,3aRS)-8-Fluoro-3-phenyl-3a,4-dihydro-3H-chromeno[4,3-c]pyrazole-2-carboxylic acid methyl-(2-methylamino-ethyl)-amide,

(24) (3RS,3aRS)-8-Fluoro-3-phenyl-3a,4-dihydro-3H-chromeno[4,3-c]pyrazole-2-carboxylic acid methyl-(1-methyl-piperidin-3-ylmethyl)-amide with (R)— or (S)-methyl-piperidin-3-yl or mixtures thereof,

(25) (3RS,3aRS)-8-Fluoro-3-phenyl-3a,4-dihydro-3H-chromeno[4,3-c]pyrazole-2-carboxylic acid-methyl-piperidin-3-yl-amide with (R)— or (S)-methyl-piperidin-3-yl or mixtures thereof,

(26) (3RS,3aRS)-8-Fluoro-3-phenyl-3a,4-dihydro-3H-chromeno[4,3-c]pyrazole-2-carboxylic acid (-1-isopropyl-piperidin-3-ylmethyl)-methyl-amide with (R)— or (S)-1-isopropyl-piperidin-3-yl or mixtures thereof,

(27) (3RS,3aRS)-8-Fluoro-3-(3-hydroxy-phenyl)-3a,4-dihydro-3H-chromeno[4,3-c]pyrazole-2-carboxylic acid methyl-piperidin-4-yl-amide,

(28) (3RS,3aRS)-8-Fluoro-3-phenyl-3a,4-dihydro-3H-chromeno[4,3-c]pyrazole-2-carboxylic acid methyl-(2-pyrrolidin-1-yl-ethyl)-amide,

(29) ((3RS,3aRS)-8-Fluoro-3-phenyl-3a,4-dihydro-3H-chromeno[4,3-c]pyrazole-2-carboxylic acid [2-(4-acetyl-piperazin-1-yl)-ethyl]-methyl-amide,

(30) 4-{[1-((3RS,3aRS)-8-Fluoro-3-phenyl-3,3a,4,5-tetrahydro-pyrazolo[4,3-c]quinolin-2-yl)-carbonyl]-methyl-amino}-piperidine-1-carboxylic acid tert-butyl ester,

(31) (2-{[1-((3RS,3aRS)-8-Fluoro-3-phenyl-3a,4-dihydro-3H-chromeno[4,3-c]pyrazol-2-yl)-carbonyl]-methyl-amino}-ethyl)-carbamic acid tert-butyl ester,

(32) (3-{[1-((3RS,3aRS)-8-Fluoro-3-phenyl-3a,4-dihydro-3H-chromeno[4,3-c]pyrazol-2-yl)-carbonyl]-methyl-amino}-propyl)-carbamic acid tert-butyl ester,

(33) (S AND R)-3-{[1-((3SR,3aSR)-8-Fluoro-3-phenyl-3a,4-dihydro-3H-chromeno[4,3-c]pyrazol-2-yl)-carbonyl]-methyl-amino}-piperidine-1-carboxylic acid tert-butyl ester,

(34) (3SR,3aSR)-8-Fluoro-3-phenyl-3a,4-dihydro-3H-chromeno[4,3-c]pyrazole-2-carboxylic acid methyl-(3-pyrrolidin-1-yl-propyl)-amide

(35) (3SR,3aSR)-8-Fluoro-3-phenyl-3a,4-dihydro-3H-chromeno[4,3-c]pyrazole-2-carboxylic acid [3-(4-acetyl-piperazin-1-yl)-propyl]-methyl-amide,

(36) (3SR,3aSR)-8-Fluoro-3-phenyl-3a,4-dihydro-3H-chromeno[4,3-c]pyrazole-2-carboxylic acid methyl-(2-piperidin-1-yl-ethyl)-amide,

(37) (3SR,3aSR)-8-Fluoro-3-phenyl-3a,4-dihydro-3H-chromeno[4,3-c]pyrazole-2-carboxylic acid methyl-(3-piperidin-1-yl-propyl)-amide,

(38) (3SR,3aSR)-8-Fluoro-3-phenyl-3a,4-dihydro-3H-chromeno[4,3-c]pyrazole-2-carboxylic acid (3-azetidin-1-yl-propyl)-methyl-amide,

(39) (3SR,3aSR)-8-Fluoro-3-phenyl-3a,4-dihydro-3H-chromeno[4,3-c]pyrazole-2-carboxylic acid (2-isopropylamino-ethyl)-methyl-amide,

(40) (3SR,3aSR)-8-Fluoro-3-phenyl-3a,4-dihydro-3H-chromeno[4,3-c]pyrazole-2-carboxylic acid (2-tert-butylamino-ethyl)-methyl-amide,

(41) (3SR,3aSR)-8-Fluoro-3-phenyl-3a,4-dihydro-3H-chromeno[4,3-c]pyrazole-2-carboxylic acid (2-isobutylamino-ethyl)-methyl-amide,

(42) (3SR,3aSR)-8-Fluoro-3-phenyl-3a,4-dihydro-3H-chromeno[4,3-c]pyrazole-2-carboxylic acid (3-isobutylamino-propyl)-methyl-amide,

(43) (3SR,3aSR)-8-Fluoro-3-phenyl-3a,4-dihydro-3H-chromeno[4,3-c]pyrazole-2-carboxylic acid (3-tert-butylamino-propyl)-methyl-amide,

(44) (3SR,3aSR)-8-Fluoro-3-phenyl-3a,4-dihydro-3H-chromeno[4,3-c]pyrazole-2-carboxylic acid (2-cyclobutylamino-ethyl)-methyl-amide,

(45) (3SR,3aSR)-8-Fluoro-3-phenyl-3a,4-dihydro-3H-chromeno[4,3-c]pyrazole-2-carboxylic acid (2-diethylamino-ethyl)-methyl-amide,

(46) (3SR,3aSR)-8-Fluoro-3-phenyl-3a,4-dihydro-3H-chromeno[4,3-c]pyrazole-2-carboxylic acid (3-diethylamino-propyl)-methyl-amide,

(47) (3SR,3aSR)-8-Fluoro-3-phenyl-3a,4-dihydro-3H-chromeno[4,3-c]pyrazole-2-carboxylic acid [2-(2-methoxy-ethylamino)-ethyl]-methyl-amide,

(48) (3SR,3aSR)-8-Fluoro-3-phenyl-3a,4-dihydro-3H-chromeno[4,3-c]pyrazole-2-carboxylic acid [3-(2-methoxy-ethylamino)-propyl]-methyl-amide,

(49) (3SR,3aSR)-8-Fluoro-3-phenyl-3a,4-dihydro-3H-chromeno[4,3-c]pyrazole-2-carboxylic acid {2-[(2-hydroxy-ethyl)-methyl-amino]-ethyl}-methyl-amide,

(50) (3SR,3aSR)-8-Fluoro-3-phenyl-3a,4-dihydro-3H-chromeno[4,3-c]pyrazole-2-carboxylic acid {3-[(2-hydroxy-ethyl)-methyl-amino]-propyl}-methyl-amide,

(51) (3SR,3aSR)-8-Fluoro-3-phenyl-3a,4-dihydro-3H-chromeno[4,3-c]pyrazole-2-carboxylic acid (2-allylamino-ethyl)-methyl-amide,

(52) (3SR,3aSR)-8-Fluoro-3-phenyl-3a,4-dihydro-3H-chromeno[4,3-c]pyrazole-2-carboxylic acid (3-allylamino-propyl)-methyl-amide,

(53) (3SR,3aSR)-8-Fluoro-3-phenyl-3a,4-dihydro-3H-chromeno[4,3-c]pyrazole-2-carboxylic acid [2-(ethyl-methyl-amino)-ethyl]-methyl-amide,

(54) (3SR,3aSR)-8-Fluoro-3-phenyl-3a,4-dihydro-3H-chromeno[4,3-c]pyrazole-2-carboxylic acid [3-(cyclopropylmethyl-amino)-propyl]-methyl-amide,

(55) (3SR,3aSR)-8-Fluoro-3-phenyl-3a,4-dihydro-3H-chromeno[4,3-c]pyrazole-2-carboxylic acid [3-(ethyl-methyl-amino)-propyl]-methyl-amide,

(56) (3SR,3aSR)-8-Fluoro-3-phenyl-3a,4-dihydro-3H-chromeno[4,3-c]pyrazole-2-carboxylic acid {3-[ethyl-(2-methoxy-ethyl)-amino]-propyl}-methyl-amide,

(57) (3SR,3aSR)-8-Fluoro-3-phenyl-3a,4-dihydro-3H-chromeno[4,3-c]pyrazole-2-carboxylic acid {2-[ethyl-(2-methoxy-ethyl)-amino]-ethyl}-methyl-amide,

(58) (3RS,3aRS)-8-Fluoro-3-phenyl-3a,4-dihydro-3H-chromeno[4,3-c]pyrazole-2-carboxylic acid {2-[ethyl-(2-hydroxy-ethyl)-amino]-ethyl}-methyl-amide,

(59) (3RS,3aRS)-8-Fluoro-3-phenyl-3a,4-dihydro-3H-chromeno[4,3-c]pyrazole-2-carboxylic acid {3-[ethyl-(2-hydroxy-ethyl)-amino]-propyl}-methyl-amide,

(60) (3SR,3aSR)-8-Fluoro-3-phenyl-3a,4-dihydro-3H-chromeno[4,3-c]pyrazole-2-carboxylic acid (2-cyclopropylamino-ethyl)-methyl-amide,

(61) (3SR,3aSR)-8-Fluoro-3-phenyl-3a,4-dihydro-3H-chromeno[4,3-c]pyrazole-2-carboxylic acid [2-(2-fluoro-ethylamino)-ethyl]-methyl-amide,

(62) (3SR,3aSR)-8-Fluoro-3-phenyl-3a,4-dihydro-3H-chromeno[4,3-c]pyrazole-2-carboxylic acid (2-ethylamino-ethyl)-methyl-amide,

(63) (3SR,3aSR)-8-Fluoro-3-phenyl-3a,4-dihydro-3H-chromeno[4,3-c]pyrazole-2-carboxylic acid [2-(allyl-methyl-amino)-ethyl]-methyl-amide,

(64) (3SR,3aSR)-8-Fluoro-3-phenyl-3a,4-dihydro-3H-chromeno[4,3-c]pyrazole-2-carboxylic acid methyl-[2-(methyl-prop-2-ynyl-amino)-ethyl]-amide,

(65) (3SR,3aSR)-8-Fluoro-3-phenyl-3a,4-dihydro-3H-chromeno[4,3-c]pyrazole-2-carboxylic acid methyl-[2-(4-methyl-piperidin-1-yl)-ethyl]-amide,

(66) (3SR,3aSR)-8-Fluoro-3-phenyl-3a,4-dihydro-3H-chromeno[4,3-c]pyrazole-2-carboxylic acid methyl-[3-(4-methyl-piperidin-1-yl)-propyl]-amide,

and the salts, stereoisomers and the salts of the stereoisomers of these compounds.

The compounds according to the invention can be prepared e.g. as described as follows and according to the following specified reaction steps, or, particularly, in a manner as described by way of example in the following examples, or analogously or similarly thereto according to preparation procedures or synthesis strategies known to the person skilled in the art.

As shown in the synthesis route outlined in scheme 1 below, 6-fluoro-chroman-4-one is reacted with benzaldehydes of formula V, in which Rc is hydrogen or —OPG1, in which PG1 stands for a suitable temporary protective group for the phenolic hydroxyl group, particularly benzyl or one of those art-known protective groups mentioned in “Protective Groups in Organic Synthesis” by T. Greene and P. Wuts (John Wiley & Sons, Inc. 1999, 3^{rd} Ed.) or in “Protecting Groups (Thieme Foundations Organic Chemistry Series N Group” by P. Kocienski (Thieme Medical Publishers, 2000), using protocols known for aldol condensation reactions (see e.g. Belluti et al, J. Med. Chem 2005, 48, 4444-4456; or F. Fournier et al, Eur. J. Med. Chem. 1981, 16, 48-58) to give corresponding benzylidene compounds of formula IV.

Compounds of formula V are known or can be obtained according to known procedures.

In a first alternative, the benzylidene compounds of formula IV, in which Rc is hydrogen or —OPG1, in which PG1 stands for said temporary protective group (particularly benzyl), are reacted with hydrazine in a ring closure condensation to give corresponding compounds of formula III as a mixture of stereoisomers. This cyclocondensation reaction can be carried out analogously or similarly to known procedures or as described in the following examples and leads to compounds of formula III normally as a mixture of racemic cis/trans diastereomers (e.g. similarly to Amr et al, Bioorganic and Medicinal Chemistry 2006, 14, 373-384). Hereby, the cis and trans diastereomers are characterized in the fact, that the hydrogen atoms in positions 3 and 3a are arranged in cis or trans position, respectively, relative to one another. Said mixture can be optionally separated in a manner habitual to the skilled person or as described herein, such as e.g. diastereomeric compounds (cis/trans isomers) can be optionally separated from one another by fractional crystallization from a suitable solvent (e.g. ethanol). Both the cis and the trans diastereomers of formula III are not stable and decompose quickly.

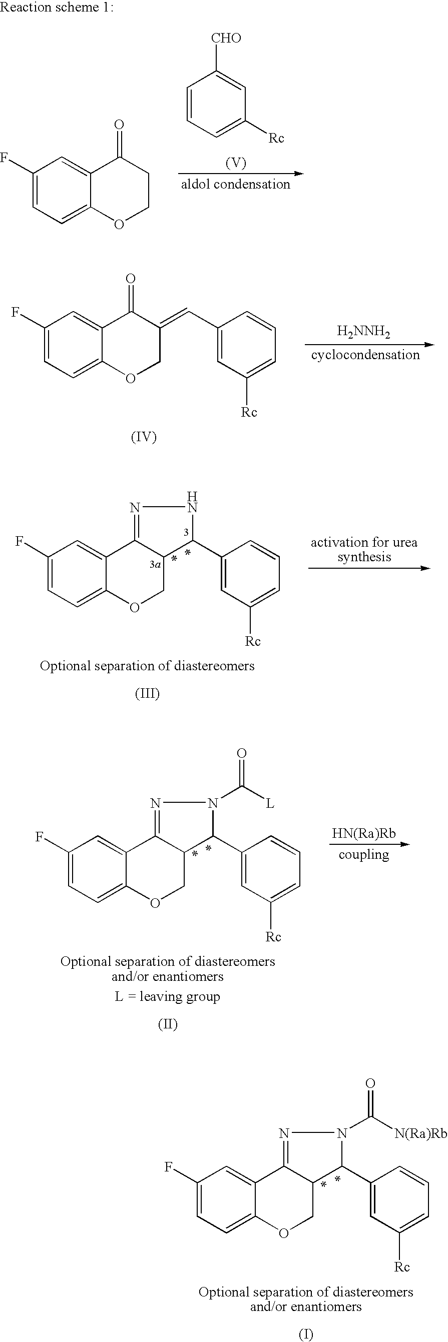

The cis derivatives of formula III, which can be prepared in the aforementioned cyclocondensation reaction, are obtained as racemate (racemic mixture) comprising the enantiomeric compounds of formulae IIIa′ and IIIb′; from which racemate those enantiomers having the formula IIIa′ are to be in particular emphasized.

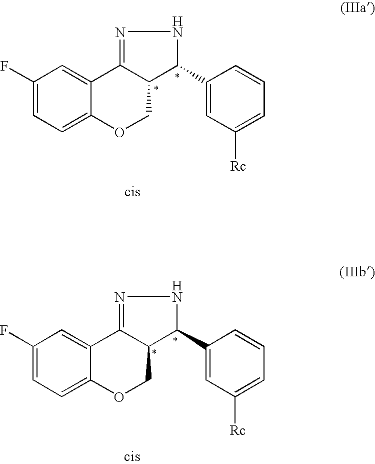

Accordingly, the trans derivatives of formula III, which can be also prepared in the aforementioned cyclocondensation reaction, are obtained as racemate (racemic mixture) comprising the enantiomeric compounds of formulae IIIa″ and IIIb″; from which racemate those compounds having the formula IIIa″ are to be emphasized.

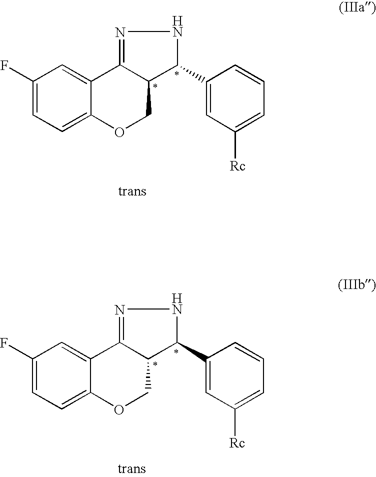

Compounds of formula III (as mixture of stereoisomers or as separate stereoisomers) can be converted into the corresponding urea compounds of formula I. This urea synthesis can be carried out similarly to known urea synthesis procedures or as described herein, such as e.g. via activated compounds of formula H, in which L is a suitable leaving group (e.g. 4-nitrophenoxy or chlorine), which are coupled with amines of formula HN(Ra)Rb in a manner customary per se to the skilled person or as described in the following examples or analogously or similarly thereto (e.g. in the presence of a suitable base like NEt₃ and, optionally, DMAP).

Thus, in more detailed example, as shown in reaction scheme 2, compounds of formulae IIIa′ and IIIa″ (in enantiomerically pure form or as racemic mixtures or as mixture of racemic diastereomers), in which Rc is hydrogen or —OPG1, in which PG1 stands for said temporary protective group (particularly benzyl), can be activated via conversion to the corresponding carbamoylchlorides of formulae IIa′ and IIa″, in which L is chlorine, by a reaction customary for the skilled person or as described in the following examples, e.g. with the aid of phosgene or triphosgene.

Alternatively to activation via carbamoylchlorides, compounds of formulae IIIa′ and IIIa″ (in enantiomerically pure form or as racemic mixtures or as mixture of racemic diastereomers), in which Rc is hydrogen or —OPG1, in which PG1 stands for said temporary protective group (particularly benzyl), may be activated by reaction with compounds of formula L-C(O)—X, in which X and L are suitable leaving groups, such as e.g. X is chlorine and L is 4-nitrophenoxy to give corresponding activated carbamates (e.g. p-nitrophenyl carbamates) of formulae IIa′ and IIa″ in which L is 4-nitrophenoxy.

Compounds of formula L-C(O)—X are known or can be obtained according to known procedures.

If stereoisomeric mixtures of compounds of formulae IIIa′ and IIIa″ are used, the resulting activated derivatives may be separated in stereoisomeric pure forms in a manner habitual to the skilled person or as described herein or analogously or similarly thereto, such as e.g. diastereomeric carbamoylchlorides (cis/trans isomers) can be separated from one another by column chromatography under suitable separation conditions using an appropriate stationary phase, as well as enantiomeric carbamoylchlorides can be separated from one another by chiral column chromatography under suitable separation conditions using an appropriate chiral stationary phase.

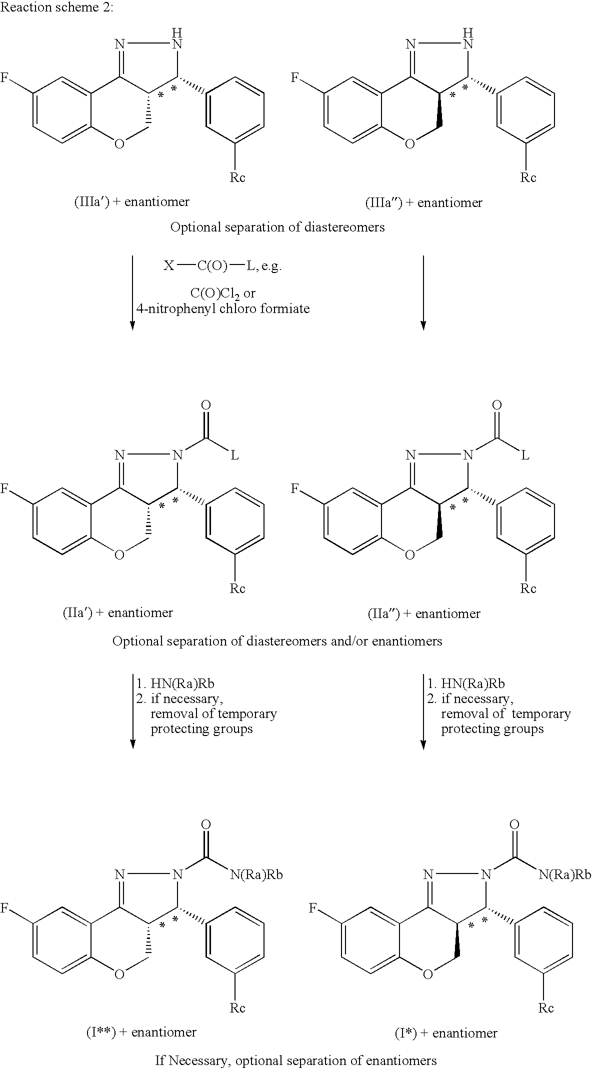

In more detail, the cis diastereomers of the mentioned carbamoylchlorides having the formulae IIa′ and IIb′ as shown below, in which L is chlorine, Rc is hydrogen or —OPG1, in which PG1 stands for said temporary protective group (particularly benzyl), are separated from the corresponding trans diastereomers having the formulae IIa″ and IIb″ as shown below.

Among compounds of formula II the cis diastereomers are worthy to be mentioned.

Yet in more detail, the cis enantiomer of the mentioned carbamoylchlorides having the formula IIa′, in which L is chlorine, Rc is hydrogen or —OPG1, in which PG1 stands for said temporary protective group (particularly benzyl), is separated from the corresponding other cis enantiomer having the formula IIb′, e.g. by chromatographic separation on chiral separating columns.

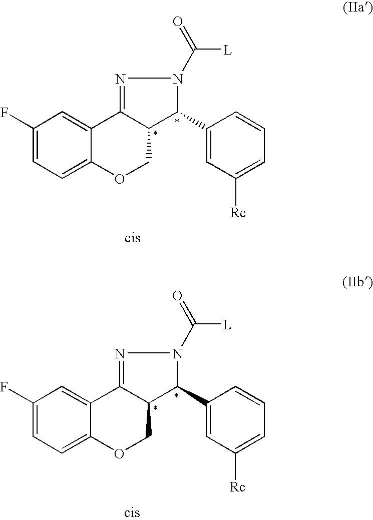

Among compounds of formulae IIa′ and IIb′ those enantiomers having the formula IIa′ are to be in particular emphasized.

Likewise, the trans enantiomer of the mentioned carbamoylchlorides having the formula IIa″, in which L is chlorine, Rc is hydrogen or —OPG1, in which PG1 stands for said temporary protective group (particularly benzyl), is separated from the corresponding other trans enantiomer having the formula IIb″.

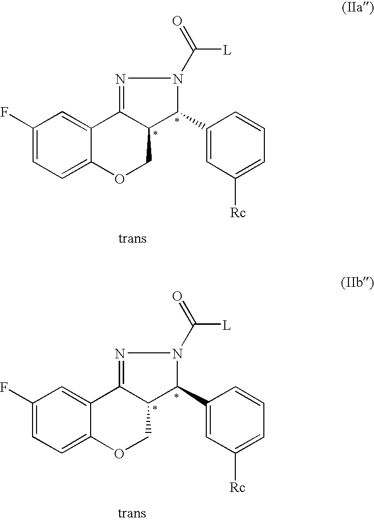

Among compounds of formulae IIa″ and IIb″ those enantiomers having the formula IIa″ are to be emphasized.

The carbamoylchlorides of formula IIa′ or IIa″ (in enantiomerically pure form or as racemic mixtures or as mixtures of racemic diastereomers), in which L is chlorine, Rc is hydrogen or —OPG1, in which PG1 stands for said temporary protective group (particularly benzyl), are reacted with amines of formula HN(Ra)Rb, in which Ra and Rb stand for the groups given above, which —if necessary—can be temporarily protected by appropriate protecting groups (such as e.g. free amino functions can be temporarily protected by the tert-butyloxycarbonyl (Boc) protecting group), to give in a manner customary for the skilled person or as described in the following examples corresponding compounds of formula I* (from compounds of formula IIa″) or I** (from compounds of formula IIa′).

Likewise, activated compounds of formula IIa′ or IIa″ (in enantiomerically pure form or as racemic mixtures or as mixtures of racemic diastereomers), in which L is a suitable leaving group (particularly 4-nitrophenoxy), Rc is hydrogen or —OPG1, in which PG1 stands for said temporary protective group (particularly benzyl), may be reacted with amines of formula HN(Ra)Rb, in which Ra and Rb stand for the groups given above, which —if necessary—can be temporarily protected by appropriate protecting groups (such as e.g. free amino functions are temporarily protected by the tert-butyloxycarbonyl (Boc) protecting group), to give in a manner customary for the skilled person corresponding compounds of formula I* (from compounds of formula IIa″) or I** (from compounds of formula IIa′).

Compounds of formula HN(Ra)Rb are known or can be obtained according to known procedures.

For example, secondary amines of formula HN(Ra)Rb can be obtained from the corresponding primary amines of formula H₂N(Ra) or H₂N(Rb) by art-known procedures, e.g. by reductive amination reaction of the corresponding aldehydes or ketones of formula (Rb)═O or (Ra)═O with the corresponding amines of formula H₂N(Ra) or H₂N(Rb), respectively.

Thus, e.g. compounds of formula HN(HetA)Rb, in which HetA and Rb have the meanings given above, may be prepared as described in WO2005019206 or WO2005017190, the disclosures of which are incorporated herein, or as outlined in reaction scheme 3, or analogously or similarly thereto, for example by reductive amination of a corresponding ketone of the formula (HetA)=O with a corresponding amine of the formula H₂N—Rb.

Accordingly, as shown in reaction scheme 3, ketones of formula XI, in which Y is oxygen or N(PG2), wherein PG2 is a suitable temporary protecting group for the nitrogen atom, e.g. benzyloxycarbonyl (Cbz), tert-butyloxycarbonyl (Boc) or the like, Z is hydrogen, fluorine or fluoromethyl, and M —with inclusion of X—constitutes a tetrahydropyrane, tetrahydrofurane, 1N-(PG2)-piperidine or 1N-(PG2)-pyrrolidine ring, may be reductively aminated with amines of formula H₂N—Rb, in which Rb has the meanings given above, to obtain corresponding compounds of formula X. Compounds of formula X, in which Y is oxygen or N(PG2) and Z is hydrogen, fluorine or fluoromethyl, may be used for coupling reaction with activated compounds of formula II to give —after removal of temporary protecting groups—corresponding compounds of formula I, in which Ra is optionally substituted by fluorine or fluoromethyl and is tetrahydropyranyl, tetrahydrofuranyl, 1N—(H)-piperidinyl or 1N—(H)-pyrrolidinyl. Compounds of formula X, in which Y is N(PG2) and Z is hydrogen, fluorine or fluoromethyl, may be also converted into compounds of formula VI, in which Z is N(R10), wherein R10 is different from hydrogen. For this purpose, compounds of formula X, in which Y is N(PG2), may be protected with a suitable temporary protecting group PG3 (e.g. Boc), in whose presence PG2 can be removed, to give corresponding compounds of formula VIII, which may be converted into corresponding compounds of formula VII, in which R10 has the meanings given above, by introduction of R10 via alkylation or reductive amination reaction. Finally, deprotection by removal of PG3 may lead to corresponding compounds of formula VI, in which R10 is different from hydrogen and has the meanings given above, which may be used for coupling reaction with activated compounds of formula II to give —after removal of temporary protecting groups—corresponding compounds of formula I, in which Ra is optionally substituted by fluorine or fluoromethyl and is tetrahydropyranyl, tetrahydrofuranyl, 1N—(R10)-piperidinyl or 1N—(R10)-pyrrolidinyl, in which R10 is different from hydrogen and has the meanings given above.

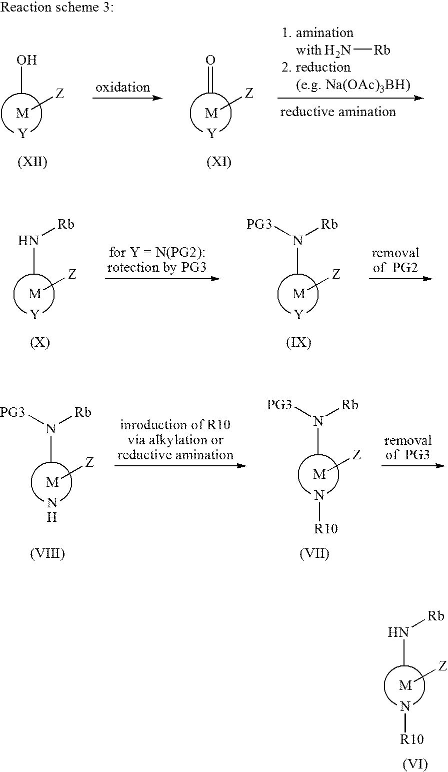

Compounds of formula XI, in which Z is fluorine, may be obtained from the corresponding compounds of formula XI, in which Z is hydrogen, by fluorination reaction, such as e.g. the fluorine atom may be incorporated in alpha position to the keto group of compounds of formula XIii by reaction of the corresponding silyl enolethers with an appropriate fluorinating reagent, e.g. Selectfluor, to give corresponding compounds of formula XIi.

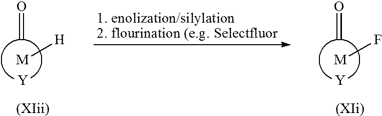

Relevant compounds of any of the formulae XII, XI, IX and VII, in each of which Z is fluoromethyl, may be obtained from the respective corresponding compounds of any of the formulae XII, XI, IX and VII, in each of which Z is hydroxymethyl, by nucleophilic substitution reaction either directly using e.g. (diethylamino)sulfur trifluoride (DAST) or via activation of the hydroxyl group with a suitable leaving group (using e.g. mesylchloride) and replacement with fluorine using e.g. tetrabutylammonium fluoride (TBAF) (advanatageously the free hydroxyl group of compounds of formula XII may be temporarily protected in these reactions).

Relevant compounds of any of the formulae XII, IX and VII, in each of which Z is hydroxymethyl, are known or may be obtained according to known procedures, e.g. from the respective corresponding compounds of any of the formulae XII, XI, IX and VII, in each of which Z is methoxycarbonyl, by reduction reaction (using e.g. LiAlH₄) (advanatageously the free hydroxyl group of compounds of formula XII may be temporarily protected in this reaction).

Relevant compounds of any of the formulae VI to XI, in each of which Z is methoxycarbonyl, may be obtained as shown in reaction scheme 3 starting from corresponding compounds of formula XII, or they are known or may be obtained according to known procedures.

Relevant compounds of formula XII, in which Z is methoxycarbonyl, are known or may be obtained according to known procedures.

Relevant compounds of formula XI, in which Z is hydrogen, fluorine, hydroxymethyl or methoxycarbonyl, are known or may be obtained according to known procedures, e.g. from the corresponding alcohols of formula XII by oxidation reaction (advanatageously the free primary hydroxyl group of compounds of formula XII, wherein Z is hydroxymethyl, may be temporarily protected in this reaction).

Thus, in more detail, (3RS,4SR)-3-Fluoro-N,1-dimethylpiperidin-4-amine and (3RS,4RS)-3-Fluoro-N,1-dimethylpiperidin-4-amine, as well as the pure enantiomers (3R,4S)-3-Fluoro-N,1-dimethylpiperidin-4-amine, (3S,4R)-3-Fluoro-N,1-dimethylpiperidin-4-amine, (3R,4R)-3-Fluoro-N,1-dimethylpiperidin-4-amine and (3S,4S)-3-Fluoro-N,1-dimethylpiperidin-4-amine are described in WO2005/019206, the disclosure of which is incorporated herein.

Yet thus, in more detail, (3RS,4RS)—N-Boc-trans-3-fluoro-4-(methylamino)-pyrrolidine is described in WO2005/017190, the disclosure of which is incorporated herein.

Yet thus, in more detail, tert-Butyl[2RS,4RS)-2-(fluoromethyl)-1-methylpiperidin-4-yl]methylcarbamate is described in WO2005/019206, the disclosure of which is incorporated herein.

Yet thus, in more detail, a synthesis route to (2-Fluoromethyl)-1-methyl-pyrrolidin-3-yl)-methyl-amine is outlined in WO2005/017190, the disclosure of which is incorporated herein.

In a second alternative, as shown in reaction scheme 4, the benzylidene compounds of formula IV, in which Rc is hydrogen or —OPG1, in which PG1 stands for said temporary protective group, are reacted with semicarbazides of formula H₂N—NH—C(O)—N(Ra)Rb, in which Ra and Rb stand for the groups given above, which —if necessary—can be temporarily protected by appropriate protecting groups, in a ring closure condensation to give corresponding compounds of formulae Ia and Ib as a mixture. This cyclocondensation reaction can be carried out analogously or similarly to known procedures and leads to compounds of formulae I* and I** normally as a mixture of racemic cis/trans diastereomers (e.g. similarly to Lorand et al, European Journal of Medicinal Chemistry 1999, 34, 1009-1018).

Compounds of formula H₂N—NH—C(O)—N(Ra)Rb are known or can be obtained according to known procedures.

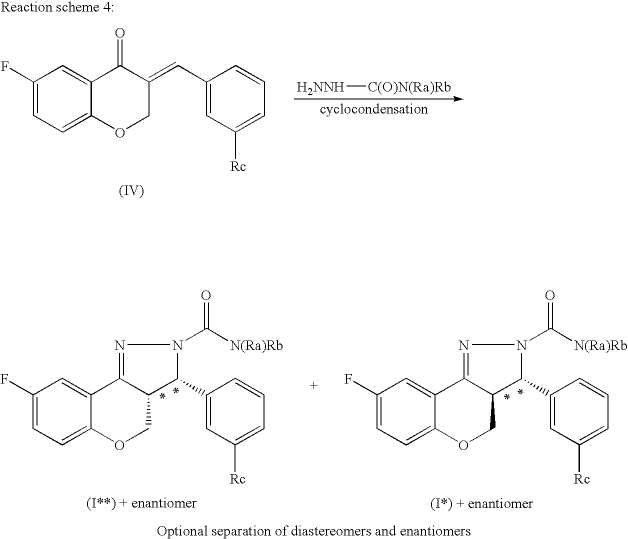

In a third alternative, as shown in reaction scheme 5, activated compounds of formula II as defined above may be coupled with primary amines of formula H₂N—Ra or azides of formula N₃-Ra, in which Ra has the meanings given above, which —if necessary—can be temporarily protected by appropriate protecting groups (such as e.g. free amino functions are temporarily protected by the tert-butyloxycarbonyl (Boc) protecting group), to give in a manner customary for the skilled person corresponding compounds of formula XIII (see e.g. in analogy as described in WO2005/017190 or WO2005/019206). Compounds of formula III may be alkylated in a manner habitual for the skilled person (see e.g. in analogy as described in WO2005/017190 or WO2005/019206) with compounds of formula Rb—X, in which Rb has the meanings given above (particularly methyl) and X is a suitable leaving group (e.g. chlorine, bromine or, particularly, iodine) in the presence of a suitable base, e.g. sodium hydride, to give corresponding compounds of formula I.

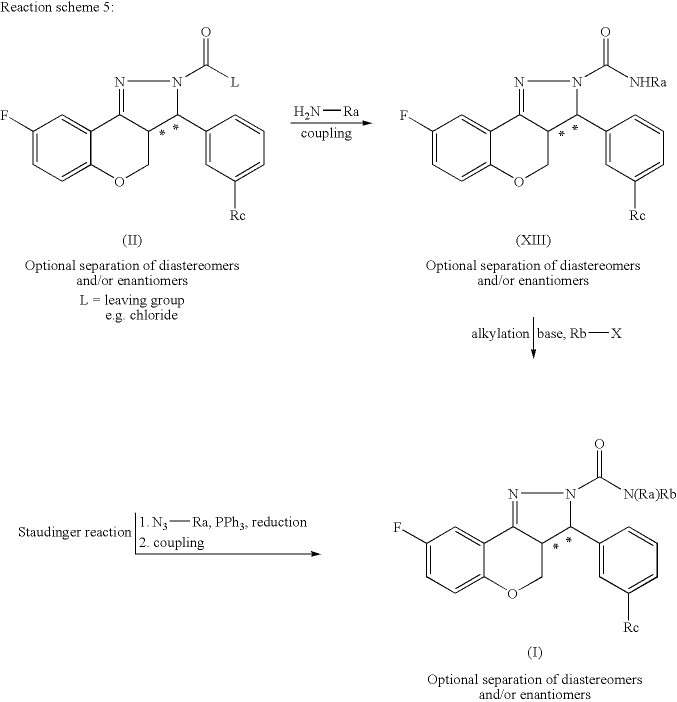

Amines of formula H₂N—Ra or azides of formula N₃—Ra are known or may be obtained according to known procedures, e.g. as described in WO2005019206 or WO2005017190, the disclosures of which are incorporated herein, or analogously or similarly thereto.

Thus, in more detail, (2S,4S)-4-Azido-2-fluoromethyl-pyrrolidine-1-carboxylic acid tert-butyl ester or (2R,4S)-4-Azido-2-fluoromethyl-pyrrolidine-1-carboxylic acid tert-butyl ester can be prepared according to Rosen et al., J. Med. Chem. 1988, 31, 1598-1611 from (2S,4R)-4-hydroxy-L-proline ethyl ester or (2R,4R)-4-hydroxy-D-proline methyl ester, respectively.

Yet thus, in more detail, a synthesis route to (3R,4R)-3-Amino-4-fluoro-pyrrolidine-1-carboxylic acid benzyl ester or (3R,4S)-3-Amino-4-fluoro-pyrrolidine-1-carboxylic acid benzyl ester is outlined in WO2005/017190, the disclosure of which is incorporated herein.

Yet thus, in more detail, a synthesis route to 4-Amino-2-fluoromethyl-pyrrolidine-1-carboxylic acid tert-butyl ester is outlined in WO2005/017190, the disclosure of which is incorporated herein.

Compounds of formula HN(Ra)Rb, H₂N—Ra or N₃—Ra, which are chiral, may be used as mixtures of stereoisomers, such as e.g. as racemic mixtures (and separation of resulting stereoisomers may be carried out at the stage of compounds of formula I), or as pure stereoisomers, which may be obtained according to art-known procedures, such as e.g. diastereomers may be separated by column chromatography or fractional crystallization and enantiomers may be separated by chiral column chromatography or diastereomeric salt formation with optically active acids.

In a fourth alternative, compounds of formula I, in which Rb has the meanings given above, Rc is hydrogen or —OPG1, in which PG1 stands for a suitable temporary protective group (particularly benzyl), and Ra is 1-4C-alkyl (advantageously 2-4C-alkyl) substituted by X, in which X is a suitable leaving group, e.g. chlorine or bromine, can be reacted in a nucleophilic substitution reaction with amines of formula HN(R2)R3, in which R2 and R3 stand for the groups given above, which —if necessary—can be temporarily protected by appropriate protecting groups (such as e.g. free amino functions can be temporarily protected by the tert-butyloxycarbonyl (Boc) protecting group), to prepare corresponding compounds of formula I, in which Ra is 1-4C-alkyl substituted by —N(R2)R3.

In more detail, as shown in reaction scheme 6, compounds of formula XIV, in which Rb has the meanings given above, Rc is hydrogen or —OPG1, in which PG1 stands for a suitable temporary protective group (particularly benzyl), n is 1 or 2, and X is a suitable leaving group, e.g. chlorine, are reacted with the amines of formula HN(R2)R3 in a manner habitual per se for the skilled person or as described in the following examples or analogously or similarly thereto to obtain corresponding compounds of formula Ii. Optionally this nucleophilic substitution reaction can be carried out in the presence of microwaves.

The compounds of formula XIV can be obtained by coupling of corresponding activated compounds of formula II and corresponding amines of formula HN(Rb)—CH₂—(CH₂)ₙ—X as described herein.

Amines of formula HN(Rb)—CH₂—(CH₂)ₙ—X are known or can be prepared according to known procedures.

Amines of formula HN(R2)R3 are known or can be prepared according to known procedures or similarly as described hereinabove for amines of formula HN(Ra)Rb.

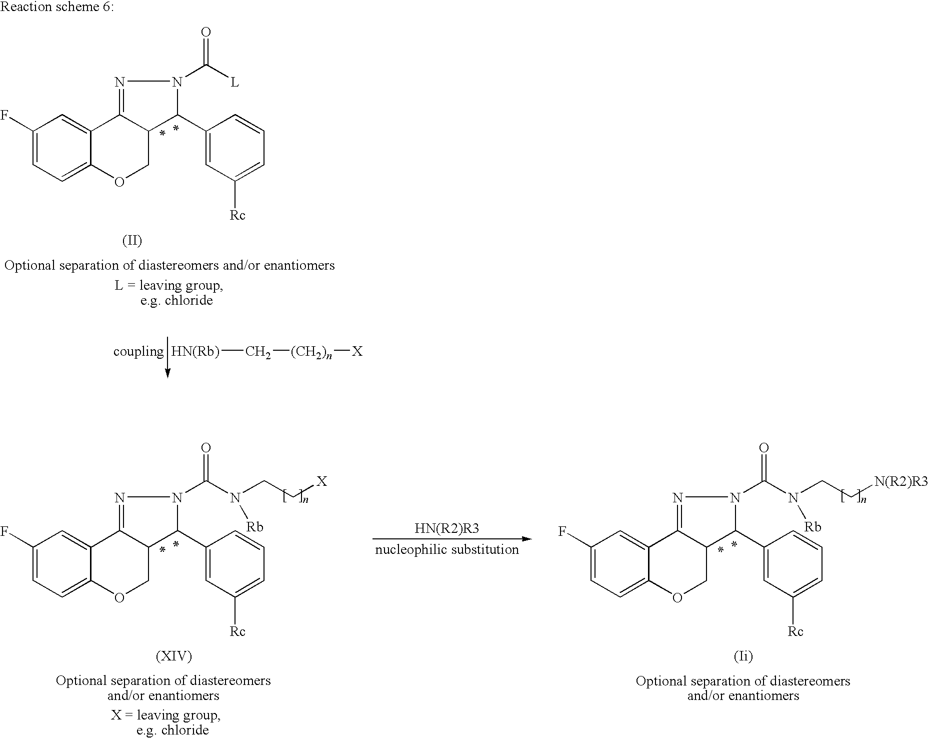

If necessary, compounds of formula I, I* or I** (in enantiomerically pure form or as racemic mixtures or as mixture of racemic diastereomers), which are protected by temporary protecting groups as defined above, are deprotected by removal of said protecting groups in a manner known to the person skilled in the art or as described in the following examples or analogously or similarly thereto to give corresponding unprotected compounds of formula I, I* or I**, in which Ra, Rb and Rc have the meanings indicated above at the outset.

In more detail, compounds of formula I, I* or I**, in which Rc is benzyloxy, are debenzylated in an art-known manner or as described in the following examples, and/or compounds of formula I, I* or I**, in which Ra and Rb stand for the groups given above, whose free amino functions are protected by the Boc protecting group, are deprotected by art-known removal of Boc to give corresponding deprotected compounds of formula I, I* or I**.

It is to be understood for the skilled worker, that certain compounds according to this invention may be converted into further compounds of this invention by art-known synthesis strategies and reactions habitual per se to a person of ordinary skill in the art.

Therefore, optionally, compounds of formula I can be converted into further compounds of formula I by methods known to one of ordinary skill in the art. More specifically, for example, from compounds of the formula I in which
a) R10 or R21 is hydrogen, the corresponding N-alkylated compounds can be obtained by reductive amination or nucleophilic substitution reaction;
b) R10 or R21 is hydrogen, the corresponding N-acylated compounds can be obtained by acylation reaction;
c) R2 and/or R3 are hydrogen, the corresponding N-alkylated compounds can be obtained by reductive amination or nucleophilic substitution reaction.
d) Raa is chlorine or bromine, the corresponding compounds, in which Raa is —N(R2)R3, can be obtained by nucleophilic substitution reaction with amines of formula HN(R2)R3.

The methods mentioned under a) to d) can be expediently carried out analogously to the methods known to the person skilled in the art or as described by way of example in the following examples.

In general, mixtures of compounds of formulae I* and I** and their enantiomers can be separated in a manner habitual to the skilled person or as described herein or analogously or similarly thereto, such as, for example, diastereomeric compounds (cis/trans isomers) can be separated from one another by column chromatography or fractional crystallization from a suitable solvent, and enantiomeric compounds can be separated from one another by one of those racemate resolution methods mentioned above, e.g. column chromatograpy on a suitable chiral support material, such as e.g. described in the following examples or analogously or similarly thereto, using the appropriate separation conditions.

In more detail, a racemic mixture containing an enantiomer having the formula I*, in which Rc is hydrogen or —OH, may be resoluted under suitable separation conditions, e.g. using an appropriate chiral separation column (e.g. CHIRALPAK), to give an enantiomerically pure compound of formula I*.

Yet in more detail, a racemic mixture containing an enantiomer having the formula I**, in which Rc is hydrogen or —OH, is resoluted under suitable separation conditions, e.g. using an appropriate chiral separation column (e.g. CHIRALPAK), such as e.g. according to the procedures described in the following examples, to give an enantiomerically pure compound of formula I**.

Optionally, compounds of the formula I can be converted into their salts, or, optionally, salts of the compounds of the formula I can be converted into the free compounds. Corresponding processes are customary for the skilled person.

When one of the final steps or purification is carried out under the presence of an inorganic or organic acid (e.g. hydrochloric, trifluoroacetic, acetic or formic acid or the like), the compounds of formula I may be obtained —depending on their individual chemical nature and the individual nature of the acid used—as free base or containing said acid in an stoechiometric or non-stoechiometric quantity. The amount of the acid contained can be determined according to art-known procedures, e.g. by titration.

It is moreover known to the person skilled in the art that if there are a number of reactive centers on a starting or intermediate compound it may be necessary to block one or more reactive centers temporarily by protective groups in order to allow a reaction to proceed specifically at the desired reaction center. A detailed description for the use of a large number of proven protective groups is found, for example, in “Protective Groups in Organic Synthesis” by T. Greene and P. Wuts (John Wiley & Sons, Inc. 1999, 3^{rd} Ed.) or in “Protecting Groups (Thieme Foundations Organic Chemistry Series N Group” by P. Kocienski (Thieme Medical Publishers, 2000).

The substances according to the invention are isolated and purified in a manner known per se, for example by distilling off the solvent under reduced pressure and recrystallizing the residue obtained from a suitable solvent or subjecting it to one of the customary purification methods, such as, for example, column chromatography on a suitable support material.

Salts can be obtained by dissolving the free compound in a suitable solvent (e.g. a ketone, such as acetone, methyl ethyl ketone or methyl isobutyl ketone, an ether, such as diethyl ether, tetrahydrofuran or dioxane, a chlorinated hydrocarbon, such as methylene chloride or chloroform, or a low-molecular-weight aliphatic alcohol, such as methanol, ethanol or isopropanol) which contains the desired acid or base, or to which the desired acid or base is then added. The salts can be obtained by filtering, reprecipitating, precipitating with a nonsolvent for the addition salt or by evaporating the solvent. Salts obtained can be converted into the free compounds, which can in turn be converted into salts, by alkalization or by acidification. In this manner, pharmacologically and/or pharmaceutically unacceptable salts can be converted into pharmacologically and/or pharmaceutically acceptable salts.

Suitably, the conversions mentioned in this invention can be carried out analogously or similarly to methods which are familiar per se to the person skilled in the art.

The person skilled in the art may know on the basis of his/her knowledge and on the basis of those synthesis routes, which are shown and described within the description of this invention, how to find other possible synthesis routes for compounds according to this invention. All these other possible synthesis routes are also part of this invention.

Thus, the invention relates to a process for preparing a compound according to the invention, comprising at least one of the steps

(i) conversion of a compound of formula IIIa′ or IIIa″ and their enantiomers into compounds of formula I** or I* and their enantiomers, wherein Ra, Rb and Rc have the meanings as indicated for the compounds according to the invention

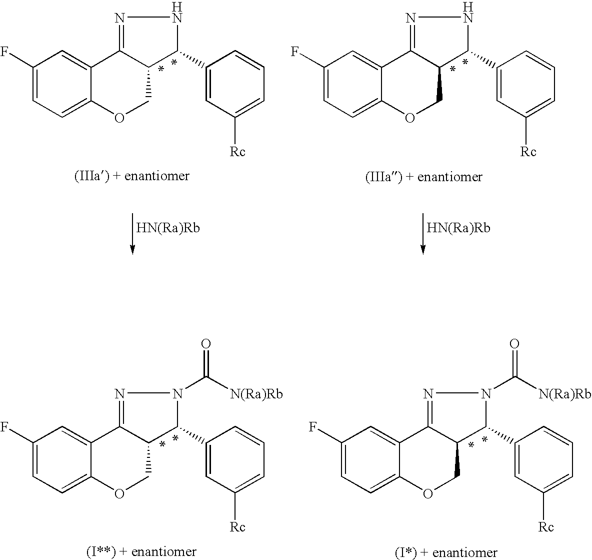

(ii) the conversion according to (i), wherein the compound of formula IIIa′ or IIIa″ and their enantiomers are activated via conversion to the corresponding carbamoylchlorides of formulae IIa′ and IIa″, in which L is chlorine, or via conversion to the corresponding activated carbamates, in which L is 4-nitrophenoxy, and in which Rc has the meanings as indicated for the compounds according to the invention,

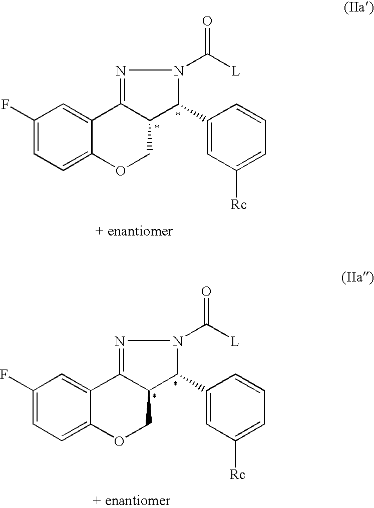

(iii) the conversion according to (i) or (ii), wherein the diastereomers or the enantiomers or both are separated, or

(iv) the conversion according to (i), (ii) or (iii), optionally followed by the removal of at least one temporary protective group selected from (a) a temporary protective group —OPG1 protecting Rc, wherein the removal comprises the conversion from Rc=-OPG1 to Rc=hydroxyl, or (b) a temporary protective group protecting an amino group that is part of the substituent Ra, wherein Ra has the meanings as indicated for the compounds according to the invention,

(v) the conversion of a compound according to the invention into a corresponding salt of the compound according to the invention.

The present invention also relates to intermediates (including their salts, stereosiomers as well as salts of these stereoisomers), methods and processes useful in synthesizing compounds according to this invention.

Having described the invention in detail, the scope of the present invention is not limited only to those described characteristics or embodiments. As will be apparent to persons skilled in the art, modifications, analogies, variations, derivatizations, homologisations and adaptations to the described invention can be made on the base of art-known knowledge and/or, particularly, on the base of the disclosure (e.g. the explicite, implicite or inherent disclosure) of the present invention without departing from the spirit and scope of this invention as defined by the appended claims.

The following examples illustrate the invention in greater detail without restricting it. Likewise, further compounds according to this invention, of which the preparation is not explicitly described, can be prepared in an analogous or similar manner or in a manner familiar per se to the person skilled in the art using customary process techniques.

Any or all of the compounds of formula I according to the present invention which are mentioned as final compounds in the following examples, as well as the salts, stereoisomers and salts of the stereoisomers thereof, are a preferred subject of the present invention.

In the examples, m.p. stands for melting point, h for hour(s), min for minutes, conc. for concentrated, calc. for calculated, fnd. for found, EF for elemental formula, MS for mass spectrometry, M for molecular ion in mass spectrometry, and other abbreviations have their meanings customary per se to the skilled person.

The 2-carbonyl group (urea carbonyl) may be designated in the systematic name as carbonyl group or alternatively, as a methanoyl group.

Further on, according to common practice in stereochemistry, the term “(RS)” characterizes a racemate comprising the one enantiomer having the configuration R and the other enantiomer having the configuration S; each of these enantiomers and their salts in pure form as well as their mixtures including the racemic mixtures is part of this invention.

Yet further on, according to common practice in stereochemistry, when more than one chiral center is present in a molecule, the symbols RS and SR are used to denote the specific configuration of each of the chiral centers of a racemate. In more detail, for example, the term “(3RS,3aRS)” stands for a racemate (racemic mixture) comprising the one enantiomer having the configuration (3R,3aR) and the other enantiomer having the configuration (3S,3aS), or the term “(3RS,3aSR)” stands for a racemate (racemic mixture) comprising the one enantiomer having the configuration (3R,3aS) and the other enantiomer having the configuration (3S,3aR); each of these enantiomers and their salts in pure form as well as their mixtures including the racemic mixtures is part of this invention.

Thus, the cis-configured racemate is described as (3RS,3aRS) or, in an equivalent manner, as (3SR,3aSR) and contains the compound with the configuration (3S,3aS) as depicted in formula I** above and its enantiomer with the configuration (3R,3aR).

Accordingly, the trans-configured racemate is described as (3RS,3aSR) or, in an equivalent manner, as (3SR,3aRS) and contains the compound with the configuration (3S,3aR) as depicted in formula I* above and its enantiomer with the configuration (3R,3aS).

### Examples

Final Compounds

### 1. (3RS,3aSR)-8-Fluoro-3-(3-hydroxy-phenyl)-3a,4-dihydro-3H-chromeno[4,3-c]pyrazole-2-carboxylic acid dimethylamide

To a solution of 5.95 g (3RS,3aSR)-3-(3-Benzyloxy-phenyl)-8-fluoro-3a,4-dihydro-3H-chromeno[4,3-c]pyrazole-2-carboxylic acid dimethylamide (compound A1) in 170 ml DMF is added Pd/C. The reaction mixture is stirred at room temperature under hydrogen (atmospheric pressure). The catalyst is filtered and washed with ethyl acetate. The solvents are removed at reduced pressure. Water and ethyl acetate are added and the aqueous layer is washed with ethyl acetate. The combined organic layers are washed with water and brine and dried with magnesium sulfate. The solvent is removed at reduced pressure. After crystallization from glacial acetic acid, 2.46 g (3RS,3aSR)-8-Fluoro-3-(3-hydroxy-phenyl)-3a,4-dihydro-3H-chromeno[4,3-c]pyrazole-2-carboxylic acid dimethylamide are obtained.

m/z (MH⁺)=356.2

### 2. (3RS,3aRS)-8-Fluoro-3-(3-hydroxy-phenyl)-3a,4-dihydro-3H-chromeno[4,3-c]pyrazole-2-carboxylic acid dimethylamide

To a solution of 5.95 g (3RS,3aRS)-3-(3-Benzyloxy-phenyl)-8-fluoro-3a,4-dihydro-3H-chromeno[4,3-c]pyrazole-2-carboxylic acid dimethylamide (compound A2) in 170 ml DMF is added Pd/C. The reaction mixture is stirred at room temperature under hydrogen (atmospheric pressure). The catalyst is filtered and washed with ethyl acetate. The solvents are removed from the filtrate under reduced pressure. Water and ethyl acetate are added and the aqueous layer is washed with ethyl acetate. The combined organic layers are washed with water and brine and dried with magnesium sulfate. The solvent is removed at reduced pressure. After crystallization from glacial acetic acid, 2.46 g (3RS,3aRS)-8-Fluoro-3-(3-hydroxy-phenyl)-3a,4-dihydro-3H-chromeno[4,3-c]pyrazole-2-carboxylic acid dimethylamide are obtained.

m/z (MH⁺)=356.2

### 2a. (+)-cis-8-Fluoro-3-(3-hydroxy-phenyl)-3a,4-dihydro-3H-chromeno[4,3-c]pyrazole-2-carboxylic acid dimethylamide

Racemic (3RS,3aRS)-8-Fluoro-3-(3-hydroxy-phenyl)-3a,4-dihydro-3H-chromeno[4,3-c]pyrazole-2-carboxylic acid dimethylamide is separated into the corresponding enantiomers using a preparative HPLC system equipped with a CHIRALPAK AD-H 5 μm-250×21 mm column and carbondioxide/methanol 70/30 as the mobile phase. The title compound is the first eluted enantiomer (>99.0% ee; m/z (MH⁺)=356.1) having at Na 589 nm at 20° C. in CHCl₃ (0.2490 g/100 ml) [α]²⁰_{D}=+372°.

### 2b. (−)-cis-8-Fluoro-3-(3-hydroxy-phenyl)-3a,4-dihydro-3H-chromeno[4,3-c]pyrazole-2-carboxylic acid dimethylamide

Racemic (3RS,3aRS)-8-Fluoro-3-(3-hydroxy-phenyl)-3a,4-dihydro-3H-chromeno[4,3-c]pyrazole-2-carboxylic acid dimethylamide is separated into the corresponding enantiomers using a preparative HPLC system equipped with a CHIRALPAK AD-H 5 μm-250×21 mm column and carbondioxide/methanol 70/30 as the mobile phase. The title compound is the second eluted enantiomer (>99.5% ee; m/z (MH⁺)=356.1).

### 3. (3RS,3aRS)-8-Fluoro-3-(3-hydroxy-phenyl)-3a,4-dihydro-3H-chromeno[4,3-c]pyrazole-2-carboxylic acid methyl-(1-methyl-piperidin-4-yl)-amide

The title compound is prepared analogously to the protocol described above for the synthesis of (3RS,3aRS)-8-Fluoro-3-(3-hydroxy-phenyl)-3a,4-dihydro-3H-chromeno[4,3-c]pyrazole-2-carboxylic acid dimethylamide (example 2). In this case (3RS,3aRS)-3-(3-Benzyloxy-phenyl)-8-Fluoro-3a,4-dihydro-3H-chromeno[4,3-c]pyrazole-2-carboxylic acid methyl-(1-methyl-piperidin-4-yl)-amide (compound A3) is used instead of (3RS,3aRS)-3-(3-Benzyloxy-phenyl)-8-fluoro-3a,4-dihydro-3H-chromeno[4,3-c]pyrazole-2-carboxylic acid dimethylamide. The title compound is obtained after trituration in diethyl ether.

m/z (MH⁺)=439.2

### 3a. (+)-8-Fluoro-3-(3-hydroxy-phenyl)-3a,4-dihydro-3H-chromeno[4,3-c]pyrazole-2-carboxylic acid methyl-(1-methyl-piperidin-4-yl)-amide, salt with hydrochloric acid

Racemic (3RS,3aRS)-8-Fluoro-3-(3-hydroxy-phenyl)-3a,4-dihydro-3H-chromeno[4,3-c]pyrazole-2-carboxylic acid methyl-(1-methyl-piperidin-4-yl)-amide is separated into the corresponding enantiomers using a preparative HPLC system equipped with a CHIRALPAK AD-H 5 μm-250×20 mm column and carbondioxide/isopropanol/diethylamine 80/20/1 as the mobile phase. The title compound is the first eluted enantiomer. After removal of the solvents, the residue is dissolved in a solution of hydrochloric acid in diethyl ether. The solvent is removed under reduced pressure and the residue is triturated with light petroleum (>99.5% ee; m/z (MH⁺)=439.2).

### 3b. (−)-8-Fluoro-3-(3-hydroxy-phenyl)-3a,4-dihydro-3H-chromeno[4,3-c]pyrazole-2-carboxylic acid methyl-(1-methyl-piperidin-4-yl)-amide, salt with hydrochloric acid

The target compound is obtained in the same way as described above for (+)-8-Fluoro-3-(3-hydroxy-phenyl)-3a,4-dihydro-3H-chromeno[4,3-c]pyrazole-2-carboxylic acid methyl-(1-methyl-piperidin-4-yl)-amide. In this case, the free base is eluted as the second enantiomer (>98% ee; m/z (MH⁺)=439.2) and the hydrochloride thereof has at Na 589 nm at 20° C. in CHCl₃ (0.0640 g/100 ml) [α]²⁰_{D}=−154°.

### 4. (3RS,3aRS)-8-Fluoro-3-(3-hydroxy-phenyl)-3a,4-dihydro-3H-chromeno[4,3-c]pyrazole-2-carboxylic acid (2-dimethylamino-ethyl)-methyl-amide

The title compound is prepared analogously to the protocol described above for the synthesis of (3RS,3aRS)-8-Fluoro-3-(3-hydroxy-phenyl)-3a,4-dihydro-3H-chromeno[4,3-c]pyrazole-2-carboxylic acid dimethylamide (example 2). In this case (3RS,3aRS)-3-(3-Benzyloxy-phenyl)-8-Fluoro-3a,4-dihydro-3H-chromeno[4,3-c]pyrazole-2-carboxylic acid (2-dimethylamino-ethyl)-methyl-amide (compound A4) is used instead of (3RS,3aRS)-3-(3-Benzyloxy-phenyl)-8-fluoro-3a,4-dihydro-3H-chromeno[4,3-c]pyrazole-2-carboxylic acid dimethylamide. The title compound is obtained after lyophilization of a solution of the compound in dioxane.

m/z (MH⁺)=413.1

### 5. (3RS,3aRS)-8-Fluoro-3-(3-hydroxy-phenyl)-3a,4-dihydro-3H-chromeno[4,3-c]pyrazole-2-carboxylic acid cyclopropyl-(1-methyl-piperidin-4-yl)-amide

The title compound is prepared analogously to the protocol described above for the synthesis of (3RS,3aRS)-8-Fluoro-3-(3-hydroxy-phenyl)-3a,4-dihydro-3H-chromeno[4,3-c]pyrazole-2-carboxylic acid dimethylamide (example 2). In this case (3RS,3aRS)-3-(3-Benzyloxy-phenyl)-8-Fluoro-3a,4-dihydro-3H-chromeno[4,3-c]pyrazole-2-carboxylic acid cyclopropyl-(1-methyl-piperidin-4-yl)-amide (compound A5) is used instead of (3RS,3aRS)-3-(3-Benzyloxy-phenyl)-8-fluoro-3a,4-dihydro-3H-chromeno[4,3-c]pyrazole-2-carboxylic acid dimethylamide. After evaporation of the solvent, the residue is purified by column chromatography (silica gel; ethyl acetate/methanol/ammonia 10:0.5:0.3). The resulting oil is crystallized in diisopropylether. The target compound is obtained as a colourless solid.

m/z (MH⁺)=465.2

### 6. (3RS,3aRS)-8-Fluoro-3-(3-hydroxy-phenyl)-3a,4-dihydro-3H-chromeno[4,3-c]pyrazole-2-carboxylic acid (3-dimethylamino-propyl)-methyl-amide, salt with hydrochloric acid

The title compound is prepared analogously to the protocol described above for the synthesis of (3RS,3aRS)-8-Fluoro-3-(3-hydroxy-phenyl)-3a,4-dihydro-3H-chromeno[4,3-c]pyrazole-2-carboxylic acid dimethylamide (example 2). In this case (3RS,3aRS)-3-(3-Benzyloxy-phenyl)-8-Fluoro-3a,4-dihydro-3H-chromeno[4,3-c]pyrazole-2-carboxylic acid (3-dimethylamino-propyl)-methyl-amide (compound A6) is used instead of (3RS,3aRS)-3-(3-Benzyloxy-phenyl)-8-fluoro-3a,4-dihydro-3H-chromeno[4,3-c]pyrazole-2-carboxylic acid dimethylamide. After evaporation of the solvent, the residue is purified by column chromatography (silica gel; ethyl acetate/methanol/ammonia 10:0.5:0.3). The target compound is obtained as a colourless solid.

mp: 145° C., m/z (MH⁺)=427.1

### 7. (3RS,3aRS)-8-Fluoro-3-phenyl-3a,4-dihydro-3H-chromeno[4,3-c]pyrazole-2-carboxylic acid dimethylamide

To an ice cold solution of 634 mg (3RS,3aRS)-8-Fluoro-3-phenyl-3a,4-dihydro-3H-chromeno[4,3-c]pyrazole-2-carbonyl chloride (compound A7) dissolved in 15 ml tetrahydrofuran are added 12 ml dimethyl amine (2 M in tetrahydrofuran). The mixture is stirred at room temperature over night. Ethyl acetate and hydrochloric acid (0.1 M) are added. The organic layer is dried and the solvent is removed at reduced pressure. Diisopropylether is added to the crude product. The colorless precipitate is filtered and dried. 522 mg (3RS,3aRS)-8-Fluoro-3-phenyl-3a,4-dihydro-3H-chromeno[4,3-c]pyrazole-2-carboxylic acid dimethylamide are obtained.

mp: 117° C., m/z (MH⁺)=340.1

### 8. (3RS,3aSR)-8-Fluoro-3-phenyl-3a,4-dihydro-3H-chromeno[4,3-c]pyrazole-2-carboxylic acid dimethylamide

A solution of 10 g (26.7 mmol) triphosgene in 40 ml toluene is cooled to 0° C. A solution of 7.2 g (3RS,3aSR)-6-Fluoro-3-phenyl-2,3,3a,4-tetrahydro-chromeno[4,3-c]pyrazole (trans diastereomer) in 86 ml tetrahydrofuran and 2.7 g (26.7 mmol) triethyl amine are added slowly. The mixture is stirred at room temperature for 2 h and then added slowly to an icecold solution of dimethylamine (60% in tetrahydrofuran). The resulting mixture is stirred at room temperature over night. The solvents are removed at reduced pressure and the residue is dissolved in ethyl acetate. Water is added and the aqueous layer is washed with ethyl acetate. The combined organic layers are washed with brine and dried with magnesium sulfate. The solvent is removed at reduced pressure. After column chromatography (silica gel, toluene/ethyl acetate 15:1) 4.34 g of (3RS,3aSR)-8-Fluoro-3-phenyl-3a,4-dihydro-3H-chromeno[4,3-c]pyrazole-2-carboxylic acid dimethylamide are obtained as a colorless foam.

m/z (MH⁺)=340.1

### 9. (3RS,3aRS)-8-Fluoro-3-phenyl-3a,4-dihydro-3H-chromeno[4,3-c]pyrazole-2-carboxylic acid methyl-(1-methyl-piperidin-4-yl)-amide

To a solution of 54 mg (163 μmol) (3RS,3aRS)-8-Fluoro-3-phenyl-3a,4-dihydro-3H-chromeno[4,3-c]pyrazole-2-carbonyl chloride (compound A7) in 10 ml dichloromethane is added at 0° C. a solution of 32 mg (245 μmol) 1-methyl-4-(methylamino)piperidine. The solution is stirred at room temperature over night. A solution of sodium bicarbonate is added and the organic layer is washed with dichloromethane. The combined organic layers are washed with water and dried with sodium sulfate. The solvents are removed at reduced pressure. After purification by preparative HPLC, 20.1 mg (3RS,3aRS)-8-Fluoro-3-phenyl-3a,4-dihydro-3H-chromeno[4,3-c]pyrazole-2-carboxylic acid methyl-(1-methyl-piperidin-4-yl)-amide (149 mg, 83%) are obtained as a colorless solid.

m/z (MH⁺)=423.3

### 9a. (3S,3aS)-8-Fluoro-3-phenyl-3a,4-dihydro-3H-chromeno[4,3-c]pyrazole-2-carboxylic acid methyl-(1-methyl-piperidin-4-yl)-amide

The title compound is prepared analogously to the procedure described for the synthesis of (3SR,3aSR)-8-fluoro-3-phenyl-3a,4-dihydro-3H-chromeno[4,3-c]pyrazole-2-carboxylic acid methyl-(1-methyl-piperidin-4-yl)-amide (example 9). In this case, enantiomerically pure (3S,3aS)-8-Fluoro-3-phenyl-3a,4-dihydro-3H-chromeno[4,3-c]pyrazole-2-carbonyl chloride (compound A7b) was used instead of the racemic starting material A7. MS: m/z (MH⁺)=423.0

### 10. (3RS,3aRS)-8-Fluoro-3-phenyl-3a,4-dihydro-3H-chromeno[4,3-c]pyrazole-2-carboxylic acid (2-dimethylamino-ethyl)-ethyl-amide

The title compound is prepared analogously to the procedure described for the preparation of (3RS,3aRS)-8-Fluoro-3-phenyl-3a,4-dihydro-3H-chromeno[4,3-c]pyrazole-2-carboxylic acid methyl-(1-methyl-piperidin-4-yl)-amide (example 9). In this case, N,N-dimethyl-N′-ethylethylen diamine is used instead of 1-methyl-4-(methylamino)-piperidine. The target compound is purified by preparative HPLC.

m/z (MH⁺)=411.0

### 11. (3RS,3aRS)-8-Fluoro-3-phenyl-3a,4-dihydro-3H-chromeno[4,3-c]pyrazole-2-carboxylic acid (3-dimethylamino-propyl)-methyl-amide

The title compound is prepared analogously to the procedure described for the preparation of (3RS,3aRS)-8-Fluoro-3-phenyl-3a,4-dihydro-3H-chromeno[4,3-c]pyrazole-2-carboxylic acid methyl-(1-methyl-piperidin-4-yl)-amide (example 9). In this case, N,N,N′-trimethyl -1,3-propan diamine is used instead of 1-methyl-4-(methylamino)-piperidine. The target compound is purified by preparative HPLC.

m/z (MH⁺)=411.2

### 12. (3RS,3aRS)-8-Fluoro-3-phenyl-3a,4-dihydro-3H-chromeno[4,3-c]pyrazole-2-carboxylic acid isobutyl-methyl-amide

The title compound is prepared analogously to the procedure described for the preparation of (3RS,3aRS)-8-Fluoro-3-phenyl-3a,4-dihydro-3H-chromeno[4,3-c]pyrazole-2-carboxylic acid methyl-(1-methyl-piperidin-4-yl)-amide (example 9). In this case, N-methylisobutyl amine is used instead of 1-methyl-4-(methylamino)-piperidine. The target compound is purified by column chromatography (silica gel; light benzene/ethyl acetate 6:1). The obtained oil is dissolved in dioxane and dried by lyophillization.

m/z (MH⁺)=382.1

### 13. (3RS,3aRS)-8-Fluoro-3-phenyl-3a,4-dihydro-3H-chromeno[4,3-c]pyrazole-2-carboxylic acid isopropyl-methyl-amide

The title compound is prepared analogously to the procedure described for the preparation of (3RS,3aRS)-8-Fluoro-3-phenyl-3a,4-dihydro-3H-chromeno[4,3-c]pyrazole-2-carboxylic acid methyl-(1-methyl-piperidin-4-yl)-amide (example 9). In this case, N-methylisopropyl amine is used instead of 1-methyl-4-(methylamino)-piperidine. The target compound is purified by column chromatography (silica gel; light benzene/ethyl acetate 4:1). The obtained oil is dissolved in dioxane and dried by lyophillization yielding to a colorless solid.

mp: 102-105° C.; m/z (MH⁺)=368.1

### 14. (3RS,3aRS)-8-Fluoro-3-phenyl-3a,4-dihydro-3H-chromeno[4,3-c]pyrazole-2-carboxylic acid ethyl-methyl-amide

The title compound is prepared analogously to the procedure described for the preparation of (3RS,3aRS)-8-Fluoro-3-phenyl-3a,4-dihydro-3H-chromeno[4,3-c]pyrazole-2-carboxylic acid methyl-(1-methyl-piperidin-4-yl)-amide (example 9). In this case, N-ethylmethyl amine is used instead of 1-methyl-4-(methylamino)-piperidine. The target compound is obtained after purification by column chromatography (silica gel; light benzene/ethyl acetate 3:1) as a yellowish solid.

mp: 133-137° C.; m/z (MH⁺)=354.1

### 15. (3RS,3aRS)-8-Fluoro-3-phenyl-1,3a,4,9b-tetrahydro-3H-chromeno[4,3-c]pyrazole-2-carboxylic acid cyclopropyl-(1-methyl-piperidin-4-yl)-amide

The title compound is prepared analogously to the procedure described for the preparation of (3RS,3aRS)-8-Fluoro-3-phenyl-3a,4-dihydro-3H-chromeno[4,3-c]pyrazole-2-carboxylic acid methyl-(1-methyl-piperidin-4-yl)-amide (example 9). In this case, N-cyclopropylamino-1-methyl-piperidine is used instead of 1-methyl-4-(methylamino)-piperidine. The target compound is purified by column chromatography (silica gel; ethyl acetate/methanol/ammonia 10:0.4:0.3), dissolved in dioxane and dried by lyophillization. A colourless solid is obtained.

m/z (MH⁺)=449.3

### 16. (3RS,3aRS)-8-Fluoro-3-phenyl-3a,4-dihydro-3H-chromeno[4,3-c]pyrazole-2-carboxylic acid methyl-piperidin-4-yl-amide

The title compound is prepared analogously to the procedure described for the preparation of (3RS,3aRS)-8-Fluoro-3-phenyl-3a,4-dihydro-3H-chromeno[4,3-c]pyrazole-2-carboxylic acid methyl-(1-methyl-piperidin-4-yl)-amide (example 9). In this case, 1-BOC-4-methylamino piperidine is used instead of 1-methyl-4-(methylamino)-piperidine. After purification of the BOC-protected intermediate by column chromatography (silica gel; light benzene/ethyl acetate 4:1 and as a second purification silica gel, ethyl acetate/methanol/ammonia 10:0.3:0.2), 3 ml dichloromethane and 500 μl trifluoroacetic acid are added. After full conversion (according to TLC), the mixture is washed with an aqueous solution of sodium bicarbonate and dried with magnesium sulfate. The resulting residue is dissolved in dioxane and dried by lyophillization. The target compound is obtained as a colourless solid.

mp: 196° C.; m/z (MH⁺)=409.2

### 16a. (3S,3aS)-8-Fluoro-3-phenyl-3a,4-dihydro-3H-chromeno[4,3-c]pyrazole-2-carboxylic acid methyl-piperidin-4-yl-amide

The title compound is prepared analogously to the procedure described for the synthesis of (3S,3aS)-8-fluoro-3-phenyl-3a,4-dihydro-3H-chromeno[4,3-c]pyrazole-2-carboxylic acid methyl-piperidin-4-yl-amide (example 16). In this case, enantiomerically pure (3S,3aS)-8-Fluoro-3-phenyl-3a,4-dihydro-3H-chromeno[4,3-c]pyrazole-2-carbonyl chloride (compound A7b) was used instead of the racemic starting material A7. MS: m/z (MH+)=409.0

### 17. (3RS,3aRS)-8-Fluoro-3-phenyl-3a,4-dihydro-3H-chromeno[4,3-c]pyrazole-2-carboxylic acid cyclopropyl-piperidin-4-yl-amide

The title compound is prepared analogously to the procedure described for the preparation of (3RS,3aRS)-8-Fluoro-3-phenyl-3a,4-dihydro-3H-chromeno[4,3-c]pyrazole-2-carboxylic acid methyl-(1-methyl-piperidin-4-yl)-amide (example 9). In this case, tert-butyl-(4-cyclopropylamino)-piperidine-1-carboxylate is used instead of 1-methyl-4-(methylamino)-piperidine. After evaporation of the solvent, 3 ml dichloromethane and 500 μl trifluoro acetic acid are added to the BOC-protected intermediate. After full conversion (according to TLC), the mixture is washed with an aqueous solution of sodium bicarbonate and dried with magnesium sulfate. After purification with preparative HPLC, the target compound is obtained as a colourless solid.

mp: 142° C.; m/z (MH⁺)=435.2

### 18. (3RS,3aRS)-8-Fluoro-3-phenyl-3a,4-dihydro-3H-chromeno[4,3-c]pyrazole-2-carboxylic acid (2-amino-ethyl)-methyl-amide

The title compound is prepared analogously to the procedure described for the preparation of (3RS,3aRS)-8-Fluoro-3-phenyl-3a,4-dihydro-3H-chromeno[4,3-c]pyrazole-2-carboxylic acid methyl-(1-methyl-piperidin-4-yl)-amide (example 9). In this case, N-tert-butoxycarbonyl-2-(methylamino)-ethylamine is used instead of 1-methyl-4-(methylamino)-piperidine. After purification of the BOC-protected intermediate by column chromatography (silica gel; light benzene/ethyl acetate 2:1), 10 ml dichloromethane and 500 μl trifluoroacetic acid are added. The mixture is stirred for 3 h at room temperature and washed with an aqueous solution of sodium bicarbonate and dried with magnesium sulfate. The resulting residue is dissolved in dioxane and dried by lyophillization. The target compound is obtained as a colourless solid.

mp: 113.2° C.; m/z (MH⁺)=369.2

### 18a. (3SR,3aSR)-8-Fluoro-3-phenyl-3a,4-dihydro-3H-chromeno[4,3-c]pyrazole-2-carboxylic acid (2-amino-ethyl)-methyl-amide, salt with hydrochloric acid

(2-{[1-((3RS,3aRS)-8-Fluoro-3-phenyl-3a,4-dihydro-3H-chromeno[4,3-c]pyrazol-2-yl)-carbonyl]-methyl-amino}-ethyl)-carbamic acid tert-butyl ester is prepared analogously to the procedure described for the preparation of (3RS,3aRS)-8-Fluoro-3-phenyl-3a,4-dihydro-3H-chromeno[4,3-c]pyrazole-2-carboxylic acid methyl-(1-methyl-piperidin-4-yl)-amide (example 9). In this case, (2-methylaminoethyl)-carbamic acid tert-butylester is used instead of 1-methyl-4-(methylamino)-piperidine. The BOC-protected intermediate is purified by column chromatography (silica gel; light benzene/ethyl acetate 2:1). A yellowish foam is obtained (mp: 241-247° C.; m/z (MH⁺)=469.0).

To a solution of 195 mg (2-{[1-((3RS,3aRS)-8-Fluoro-3-phenyl-3a,4-dihydro-3H-chromeno[4,3-c]pyrazol-2-yl)-carbonyl]-methyl-amino}-ethyl)-carbamic acid tert-butyl ester in 10 ml dichloromethane are added 1 ml trifluoroacetic acid. The mixture is stirred at room temperature over night. The solution is washed with diluted hydrochloric acid and with brine. The organic layer is dried with magnesium sulfate. After purification by column chromatography (silica gel, ethyl acetate/methanol/ammonia 10:0.5:0.3), a yellowish oil is obtained. The residue is dissolved in dioxane and a solution of hydrochloric acid in dioxane is added. The solvent is removed at reduced pressure. 35 mg (3RS,3aRS)-8-Fluoro-3-phenyl-3a,4-dihydro-3H-chromeno[4,3-c]pyrazole-2-carboxylic acid (2-amino-ethyl)-methyl-amide are obtained as its salt with hydrochloric acid.

m/z (MH⁺)=369.0

### 19. (3RS,3aRS)-8-Fluoro-3-phenyl-3a,4-dihydro-3H-chromeno[4,3-c]pyrazole-2-carboxylic acid (2-dimethylamino-ethyl)-methyl-amide, salt with hydrochloric acid

The title compound is prepared analogously to the procedure described for the preparation of (3RS,3aRS)-8-Fluoro-3-phenyl-3a,4-dihydro-3H-chromeno[4,3-c]pyrazole-2-carboxylic acid methyl-(1-methyl-piperidin-4-yl)-amide (example 9). In this case, N,N,N′-trimethylethylen diamine is used instead of 1-methyl-4-(methylamino)-piperidine. The target compound is purified by preparative HPLC and dissolved in dioxane. A solution of hydrochloric acid in dioxane is added and the solvents are removed at reduced pressure. The residue is crystallized in diisopropyl ether. A colourless solid is obtained.

mp: 248-250° C.; m/z (MH⁺)=397.2

### 20. (3RS,3aRS)-8-Fluoro-3-phenyl-3a,4-dihydro-3H-chromeno[4,3-c]pyrazole-2-carboxylic acid (3-amino-propyl)-methyl-amide, salt with trifluoroacetic acid

(3-{[1-((3RS,3aRS)-8-Fluoro-3-phenyl-3a,4-dihydro-3H-chromeno[4,3-c]pyrazol-2-yl)-carbonyl]-methyl-amino}-propyl)-carbamic acid tert-butyl ester is prepared analogously to the procedure described for the preparation of (3RS,3aRS)-8-Fluoro-3-phenyl-3a,4-dihydro-3H-chromeno[4,3-c]pyrazole-2-carboxylic acid methyl-(1-methyl-piperidin-4-yl)-amide (example 9). In this case, 1-BOC-amino-3-methyl aminopropan is used instead of 1-methyl-4-(methylamino)-piperidine. The BOC-protected intermediate is purified by column chromatography (silica gel; light benzene/ethyl acetate 2:1) (m/z (MH⁺)=483.0).

To a solution of 175 mg (3-{[1-((3RS,3aRS)-8-Fluoro-3-phenyl-3a,4-dihydro-3H-chromeno[4,3-c]pyrazol-2-yl)-carbonyl]-methyl-amino}-propyl)-carbamic acid tert-butyl ester in 10 ml dichloromethane are added 1 ml trifluoroacetic acid. The mixture is stirred at room temperature over night. The solvent is evaporated under reduced pressure and the residue is dissolved in a mixture of acetonitrile and water. After lyophyllization, 79 mg of (3RS,3aRS)-8-Fluoro-3-phenyl-3a,4-dihydro-3H-chromeno[4,3-c]pyrazole-2-carboxylic acid (3-amino-propyl)-methyl-amide are obtained as its salt with trifluoroacetic acid.

m/z (MH⁺)=383.0

### 21. (3RS,3aRS)-8-Fluoro-3-phenyl-3a,4-dihydro-3H-chromeno[4,3-c]pyrazole-2-carboxylic acid (1-acetyl-piperidin-4-yl)-cyclopropyl-amide

The title compound is prepared analogously to the procedure described for the preparation of (3RS,3aRS)-8-Fluoro-3-phenyl-3a,4-dihydro-3H-chromeno[4,3-c]pyrazole-2-carboxylic acid methyl-(1-methyl-piperidin-4-yl)-amide (example 9). In this case, 1-acetyl-4-cyclopropylamino piperidine is used instead of 1-methyl-4-(methylamino)-piperidine. The target compound is purified by preparative HPLC.

m/z (MH⁺)=477.1

### 22. (3RS,3aRS)-8-Fluoro-3-phenyl-3a,4-dihydro-3H-chromeno[4,3-c]pyrazole-2-carboxylic acid methyl-(3-methylamino-propyl)-amide

To a solution of 150 mg (453 μmol) (3RS,3aRS)-8-Fluoro-3-phenyl-3a,4-dihydro-3H-chromeno[4,3-c]pyrazole-2-carbonyl chloride (compound A7) in 10 ml dichloromethane is added at 0° C. a solution of 463 mg (4.54 mmol) N,N′-dimethyl-1,3-propan diamine. The solution is stirred at room temperature over night. An aqueous solution of hydrochloric acid is added and the organic layer is washed with an aqueous solution of sodium bicarbonate. The combined organic layers are washed with water and dried with sodium sulfate. The solvents are removed at reduced pressure. After purification by column chromatography (silica gel, ethyl acetate/methanol/ammonia 10:0.3:0.2), 110 mg (3RS,3aRS)-8-Fluoro-3-phenyl-3a,4-dihydro-3H-chromeno[4,3-c]pyrazole-2-carboxylic acid methyl-(3-methylamino-propyl)-amide are obtained as a colorless solid.

m/z (MH⁺)=397.1

### 23. (3RS,3aRS)-8-Fluoro-3-phenyl-3a,4-dihydro-3H-chromeno[4,3-c]pyrazole-2-carboxylic acid methyl-(2-methylamino-ethyl)-amide

The title compound is prepared analogously to the procedure described for the preparation of (3RS,3aRS)-8-Fluoro-3-phenyl-3a,4-dihydro-3H-chromeno[4,3-c]pyrazole-2-carboxylic acid methyl-(3-methylamino-propyl)-amide (example 22). In this case, N,N′-dimethylethylendiamine is used instead of N,N′-dimethyl-1,3-propan diamine.

m/z (MH⁺)=383.0

### 24. (3RS,3aRS)-8-Fluoro-3-phenyl-3a,4-dihydro-3H-chromeno[4,3-c]pyrazole-2-carboxylic acid methyl-((R)-1-methyl-piperidin-3-ylmethyl)-amide and (3RS,3aRS)-8-Fluoro-3-phenyl-3a,4-dihydro-3H-chromeno[4,3-c]pyrazole-2-carboxylic acid methyl-((S)-1-methyl-piperidin-3-ylmethyl)-amide

A mixture of the title compounds is prepared analogously to the procedure described for the preparation of (3RS,3aRS)-8-Fluoro-3-phenyl-3a,4-dihydro-3H-chromeno[4,3-c]pyrazole-2-carboxylic acid methyl-(1-methyl-piperidin-4-yl)-amide (example 9). In this case, (RS)—N-methyl-1-(1-methyl-piperidin-3-yl)methylamine is used instead of 1-methyl-4-(methylamino)-piperidine. The crude compound is purified by preparative HPLC.

m/z (MH⁺)=437.2

### 25. (3RS,3aRS)-8-Fluoro-3-phenyl-3a,4-dihydro-3H-chromeno[4,3-c]pyrazole-2-carboxylic acid (R)-methyl-piperidin-3-yl-amide and (3RS,3aRS)-8-Fluoro-3-phenyl-3a,4-dihydro-3H-chromeno[4,3-c]pyrazole-2-carboxylic acid (S)-methyl-piperidin-3-yl-amide, salt with hydrochloric acid

A mixture of 3-{(R)-[1-(3RS,3aRS)-8-Fluoro-3-phenyl-3a,4-dihydro-3H-chromeno[4,3-c]pyrazol-2-yl)-carbonyl]-methyl-amino}-piperidine-1-carboxylic acid tert-butyl ester and 3-{(S)-[1-(3RS,3aRS)-8-Fluoro-3-phenyl-3a,4-dihydro-3H-chromeno[4,3-c]pyrazol-2-yl)-carbonyl]-methyl-amino}-piperidine-1-carboxylic acid tert-butyl ester is prepared analogously to the procedure described for the preparation of (3RS,3aRS)-8-Fluoro-3-phenyl-3a,4-dihydro-3H-chromeno[4,3-c]pyrazole-2-carboxylic acid methyl-(1-methyl-piperidin-4-yl)-amide Ex31 (example 9). In this case, (RS)-1-Boc-3-methylaminopiperidine is used instead of 1-methyl-4-(methylamino)-piperidine. The intermediate is purified by column chromatography (silica gel; light petroleum, ethyl acetate 3:1) (m/z (MH⁺)=508.8).

The title compound is prepared analogously to the procedure described for the preparation of (3RS,3aRS)-8-Fluoro-3-phenyl-3a,4-dihydro-3H-chromeno[4,3-c]pyrazole-2-carboxylic acid (2-amino-ethyl)-methyl-amide hydrochloride (example 18a). In this case, a mixture of 3-{(R)-[1-(3RS,3aRS)-8-Fluoro-3-phenyl-3a,4-dihydro-3H-chromeno[4,3-c]pyrazol-2-yl)-carbonyl]-methyl-amino}-piperidine-1-carboxylic acid tert-butyl ester and 3-{(S)-[1-(3RS,3aRS)-8-Fluoro-3-phenyl-3a,4-dihydro-3H-chromeno[4,3-c]pyrazol-2-yl)-carbonyl]-methyl-amino}-piperidine-1-carboxylic acid tert-butyl ester is used instead of (2-{[1-((3RS,3aRS)-8-Fluoro-3-phenyl-3a,4-dihydro-3H-chromeno[4,3-c]pyrazol-2-yl)-carbonyl]-methyl-amino}-ethyl)-carbamic acid tert-butyl ester.

m/z (MH⁺)=409.1

### 26. (3RS,3aRS)-8-Fluoro-3-phenyl-3a,4-dihydro-3H-chromeno[4,3-c]pyrazole-2-carboxylic acid ((R)-1-isopropyl-piperidin-3-ylmethyl)-methyl-amide and (3RS,3aRS)-8-Fluoro-3-phenyl-3a,4-dihydro-3H-chromeno[4,3-c]pyrazole-2-carboxylic acid ((S)-1-isopropyl-piperidin-3-ylmethyl)-methyl-amide

The title compound is prepared analogously to the procedure described for the preparation of (3RS,3aRS)-8-Fluoro-3-phenyl-3a,4-dihydro-3H-chromeno[4,3-c]pyrazole-2-carboxylic acid methyl-(1-methyl-piperidin-4-yl)-amide Ex31 (example 9). In this case, ((RS)-1-isopropylpiperidin-3-yl-methyl)methyl amine is used instead of 1-methyl-4-(methylamino)-piperidine. The target compound is purified by column chromatography (silica gel; ethyl acetate, methanol, ammonia 10:0.2:0.1).

m/z (MH⁺)=465.2

### 27. (3RS,3aRS)-8-Fluoro-3-(3-hydroxy-phenyl)-3a,4-dihydro-3H-chromeno[4,3-c]pyrazole-2-carboxylic acid methyl-piperidin-4-yl-amide

To an ice cold solution of 2.5 g (3RS,3aRS)-3-(3-Benzyloxy-phenyl)-8-fluoro-3-phenyl-3a,4-dihydro-3H-chromeno[4,3-c]pyrazole-2-carbonyl chloride (compound A8) in 150 ml dichloromethane is added a solution of 1.62 g 1-BOC-4-methylamino piperidine in 10 ml dichloromethane. The mixture is stirred at room temperature for 2 days. A diluted aqueous solution of hydrochloric acid is added and the mixture is extracted with dichloromethane. The organic layer is washed with brine and dried with magnesium sulfate. The solvent is removed at reduced pressure and the crude intermediate is purified by column chromatography (silica gel, light petroleum/ethyl acetate 2:1).

The intermediate is dissolved in 65 ml dimethyl formamide and 1.8 g Pd/C are added. The mixture is stirred at room temperature under a hydrogen atmosphere for 15 h. The catalyst is filtered and the solvent is removed at reduced pressure. The residue is dissolved in 50 ml dichloromethane and 5 ml trifluoro acetic acid are added. The mixture is stirred at room temperature for 15 h. The solution is washed with water and the organic layer is dried with magnesium sulfate. The solvent is removed at reduced pressure. The crude product is purified by column chromatography (silica gel, ethyl acetate/methanol/ammonia 10:1:0.5). The residue is triturated in hot diisopropylether. 610 mg (42%) of a colorless solid are obtained.

m/z (MH⁺)=425.1

### 27a. (3S,3aS)-8-Fluoro-3-(3-hydroxy-phenyl)-3a,4-dihydro-3H-chromeno[4,3-c]pyrazole-2-carboxylic acid methyl-piperidin-4-yl-amide

The title compound is prepared analogously to the procedure described for the synthesis of (3S,3aS)-8-fluoro-3-(3-hydroxy-phenyl)-3a,4-dihydro-3H-chromeno[4,3-c]pyrazole-2-carboxylic acid methyl-piperidin-4-yl-amide (example 27). In this case, enantiomerically pure (3S,3aS)-3-(3-Benzyloxy-phenyl)-8-fluoro-3a,4-dihydro-3H-chromeno[4,3-c]pyrazole-2-carbonyl chloride (compound A8b) was used instead of the racemic starting material A8. MS: m/z (MH⁺)=425.0

### 28. (3RS,3aRS)-8-Fluoro-3-phenyl-3a,4-dihydro-3H-chromeno[4,3-c]pyrazole-2-carboxylic acid methyl-(2-pyrrolidin-1-yl-ethyl)-amide

To a solution of 153 mg (394 μmol) (3RS,3aRS)-8-Fluoro-3-phenyl-3a,4-dihydro-3H-chromeno[4,3-c]pyrazole-2-carboxylic acid (2-chloro-ethyl)-methyl-amide (compound A9) in 15 ml dichloromethane are added 164 mg (2.3 mmol) pyrrolidine. The mixture is heated in a microwave reactor to 85° C. for 1 h. Additional dichloromethane and a diluted aqueous solution of hydrochloric acid are added. The organic layer is dried with magnesium sulfate and the solvent is removed at reduced pressure. Diisopropylether is added to the crude product. After filtration, the target compound is obtained as a colorless solid.

MS: m/z (MH⁺)=423.1

### 29. (3RS,3aRS)-8-Fluoro-3-phenyl-3a,4-dihydro-3H-chromeno[4,3-c]pyrazole-2-carboxylic acid [2-(4-acetyl-piperazin-1-yl)-ethyl]methyl-amide

The target compound is prepared analogously to the protocol described for the synthesis of (3RS,3aRS)-8-Fluoro-3-phenyl-3a,4-dihydro-3H-chromeno[4,3-c]pyrazole-2-carboxylic acid methyl-(2-pyrrolidin-1-yl-ethyl)-amide (example 28). In this case, 1-acetyl piperazine is used instead of pyrrolidine. The crude compound is purified by column chromatography (silica gel, ethyl acetate/methanol/ammonia 10:0.2:0.1). After lyophilization 125 mg (68%) of the desired product are obtained.

m/z (MH⁺)=480.1

Using similar procedures to those described herein above but with suitable choice of the starting materials (which are mentioned herein or which can be obtained analogously or similarly to the mentioned compounds) the following compounds of formula I or a salt thereof (e.g. a hydrochloride) may be prepared:

(3RS,3aRS)-8-Fluoro-3-(3-hydroxy-phenyl)-3a,4-dihydro-3H-chromeno[4,3-c]pyrazole-2-carboxylic acid (2-dimethylamino-ethyl)-ethyl-amide,

(3RS,3aRS)-8-Fluoro-3-(3-hydroxy-phenyl)-3a,4-dihydro-3H-chromeno[4,3-c]pyrazole-2-carboxylic acid isobutyl-methyl-amide,

(3RS,3aRS)-8-Fluoro-3-(3-hydroxy-phenyl)-3a,4-dihydro-3H-chromeno[4,3-c]pyrazole-2-carboxylic acid isopropyl-methyl-amide,

(3RS,3aRS)-8-Fluoro-3-(3-hydroxy-phenyl)-3a,4-dihydro-3H-chromeno[4,3-c]pyrazole-2-carboxylic acid ethyl-methyl-amide,

(3 RS,3aRS)-8-Fluoro-3-(3-hydroxy-phenyl)-3a,4-dihydro-3 H-chromeno[4,3-c]pyrazole-2-carboxylic acid cyclopropyl-piperidin-4-yl-amide,

(3 RS,3aRS)-8-Fluoro-3-(3-hydroxy-phenyl)-3a,4-dihydro-3 H-chromeno[4,3-c]pyrazole-2-carboxylic acid (2-amino-ethyl)-methyl-amide,

(3 RS,3aRS)-8-Fluoro-3-(3-hydroxy-phenyl)-3a,4-dihydro-3 H-chromeno[4,3-c]pyrazole-2-carboxylic acid (3-amino-propyl)-methyl-amide,

(3 RS,3aRS)-8-Fluoro-3-(3-hydroxy-phenyl)-3a,4-dihydro-3 H-chromeno[4,3-c]pyrazole-2-carboxylic acid (1-acetyl-piperidin-4-yl)-cyclopropyl-amide,

(3 RS,3aRS)-8-Fluoro-3-(3-hydroxy-phenyl)-3a,4-dihydro-3 H-chromeno[4,3-c]pyrazole-2-carboxylic acid methyl-(3-methylamino-propyl)-amide,

(3 RS,3aRS)-8-Fluoro-3-(3-hydroxy-phenyl)-3a,4-dihydro-3 H-chromeno[4,3-c]pyrazole-2-carboxylic acid methyl-(2-methylamino-ethyl)-amide,

(3 RS,3aRS)-8-Fluoro-3-(3-hydroxy-phenyl)-3a,4-dihydro-3 H-chromeno[4,3-c]pyrazole-2-carboxylic acid methyl-((R)-1-methyl-piperidin-3-ylmethyl)-amide and (3RS,3aRS)-8-Fluoro-3-(3-hydroxy-phenyl)-3a,4-dihydro-3H-chromeno[4,3-c]pyrazole-2-carboxylic acid methyl-((S)-1-methyl-piperidin-3-ylmethyl)-amide,

(3 RS,3aRS)-8-Fluoro-3-(3-hydroxy-phenyl)-3a,4-dihydro-3 H-chromeno[4,3-c]pyrazole-2-carboxylic acid (R)-methyl-piperidin-3-yl-amide and (3RS,3aRS)-8-Fluoro-3-(3-hydroxy-phenyl)-3a,4-dihydro-3H-chromeno[4,3-c]pyrazole-2-carboxylic acid (S)-methyl-piperidin-3-yl-amide,

(3 RS,3aRS)-8-Fluoro-3-(3-hydroxy-phenyl)-3a,4-dihydro-3 H-chromeno[4,3-c]pyrazole-2-carboxylic acid methyl-(2-pyrrolidin-1-yl-ethyl)-amide, and

(3 RS,3aRS)-8-Fluoro-3-(3-hydroxy-phenyl)-3a,4-dihydro-3 H-chromeno[4,3-c]pyrazole-2-carboxylic acid [2-(4-acetyl-piperazin-1-yl)-ethyl]-methyl-amide.

Using similar procedures to those described herein above but with suitable choice of the starting materials (which are mentioned herein or which can be obtained analogously or similarly to the mentioned compounds) the following compounds of formula I or a salt thereof (e.g. a hydrochloride) may be prepared:

(3RS,3aRS)-8-Fluoro-3-phenyl-3a,4-dihydro-3H-chromeno[4,3-c]pyrazole-2-carboxylic acid (3-dimethylamino-propyl)-ethyl-amide,

(3RS,3aRS)-8-Fluoro-3-phenyl-3a,4-dihydro-3H-chromeno[4,3-c]pyrazole-2-carboxylic acid methyl-(3-pyrrolidin-1-yl-propyl)-amide,

(3RS,3aRS)-8-Fluoro-3-phenyl-3a,4-dihydro-3H-chromeno[4,3-c]pyrazole-2-carboxylic acid [3-(4-acetyl-piperazin-1-yl)-propyl]-methyl-amide,

(3RS,3aRS)-8-Fluoro-3-(3-hydroxy-phenyl)-3a,4-dihydro-3H-chromeno[4,3-c]pyrazole-2-carboxylic acid (3-dimethylamino-propyl)-ethyl-amide,

(3RS,3aRS)-8-Fluoro-3-(3-hydroxy-phenyl)-3a,4-dihydro-3H-chromeno[4,3-c]pyrazole-2-carboxylic acid methyl-(3-pyrrolidin-1-yl-propyl)-amide, and

(3RS,3aRS)-8-Fluoro-3-(3-hydroxy-phenyl)-3a,4-dihydro-3H-chromeno[4,3-c]pyrazole-2-carboxylic acid [3-(4-acetyl-piperazin-1-yl)-propyl]-methyl-amide.

The following examples are prepared analogously to the protocol described for the synthesis of example 28. In each case the appropriate amine indicated in the following table is used instead of pyrrolidine. The crude compounds are purified by chromatographic methods.

| Example | | | m/z |
|---|---|---|---|
| no. | Chemical name | amine used | (MH+) |
| 30 | 4-{[1-((3RS,3aRS)-8-Fluoro-3-phenyl-3,3a,4,5-tetrahydro- pyrazolo[4,3-c]quinolin-2-yl)-carbonyl]-methyl-amino}- piperidine-1-carboxylic acid tert-butyl ester | 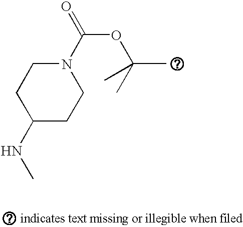 | 508.9 |
| 31 | (2-{[1-((3RS,3aRS)-8-Fluoro-3-phenyl-3a,4-dihydro-3H- chromeno[4,3-c]pyrazol-2-yl)-carbonyl]-methyl-amino}-ethyl)- carbamic acid tert-butyl ester | 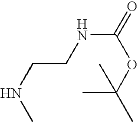 | 469.0 |
| 32 | (3-{[1-((3RS,3aRS)-8-Fluoro-3-phenyl-3a,4-dihydro-3H- chromeno[4,3-c]pyrazol-2-yl)-carbonyl]-methyl-amino}-propyl)- carbamic acid tert-butyl ester | 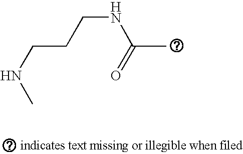 | 438.0 |
| 33 | (S AND R)-3-{[1-((3SR,3aSR)-8-Fluoro-3-phenyl-3a,4-dihydro- 3H-chromeno[4,3-c]pyrazol-2-yl)-carbonyl]-methyl-amino}- piperidine-1-carboxylic acid tert-butyl ester | 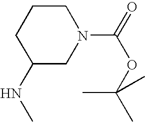 | 508.8 |

### 34. (3SR,3aSR)-8-Fluoro-3-phenyl-3a,4-dihydro-3H-chromeno[4,3-c]pyrazole-2-carboxylic acid methyl-(3-pyrrolidin-1-yl-propyl)-amide

A solution of 147 mg (365 μmol) (3SR,3aSR)-8-Fluoro-3-phenyl-3a,4-dihydro-3H-chromeno[4,3-c]pyrazole-2-carboxylic acid (3-chloro-propyl)-methyl-amide (A10) and 260 mg (3.65 mmol) pyrrolidine in 15 ml acetonitrile is heated in a sealed tube for 1 h to 100° C. using a microwave reactor. The solvent is removed under reduced pressure. Dichloromethane and a diluted aqueous solution of hydrochloric acid are added to the residue. The organic layer is dried with magnesium sulfate and the solvent is removed under reduced pressure.

The crude product was purified by column chromatography (silica gel, ethyl acetate, methanol, ammonia 10:0.3:0.2) and crystallized by the triturating with diisopropyl ether. 35 mg of the title compound are obtained. MS (MH⁺)=437.2

The following examples are prepared analogously to the protocol described for the synthesis of example 28. In each case the appropriate starting material and the appropriate amine indicated in the following table is used instead of pyrrolidine. The crude compounds are purified by chromatographic methods.

The free base of example 54 is dissolved in a solution of hydrochloric acid in dioxane, the solvent is removed under reduced pressure and the resulting solid is dried under reduced pressure to obtain the described example.

| Example | | Starting | | m/z |
|---|---|---|---|---|
| no. | Chemical name | material | Amine used | (MH⁺) |
| 35 | (3SR,3aSR)-8-Fluoro-3-phenyl-3a,4-dihydro-3H- chromeno[4,3-c]pyrazole-2-carboxylic acid [3-(4- acetyl-piperazin-1-yl)-propyl]-methyl-amide | A10 | 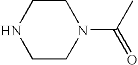 | 494.2 |
| 36 | (3SR,3aSR)-8-Fluoro-3-phenyl-3a,4-dihydro-3H- chromeno[4,3-c]pyrazole-2-carboxylic acid methyl-(2-piperidin-1-yl-ethyl)-amide | A9 | 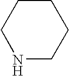 | 437.2 |
| 37 | (3SR,3aSR)-8-Fluoro-3-phenyl-3a,4-dihydro-3H- chromeno[4,3-c]pyrazole-2-carboxylic acid methyl-(3-piperidin-1-yl-propyl)-amide | A10 | 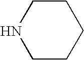 | 451.2 |
| 38 | (3SR,3aSR)-8-Fluoro-3-phenyl-3a,4-dihydro-3H- chromeno[4,3-c]pyrazole-2-carboxylic acid (3- azetidin-1-yl-propyl)-methyl-amide | A10 | 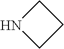 | 423.1 |
| 39 | (3SR,3aSR)-8-Fluoro-3-phenyl-3a,4-dihydro-3H- chromeno[4,3-c]pyrazole-2-carboxylic acid (2- isopropylamino-ethyl)-methyl-amide | A9 | 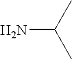 | 411.1 |
| 40 | (3SR,3aSR)-8-Fluoro-3-phenyl-3a,4-dihydro-3H- chromeno[4,3-c]pyrazole-2-carboxylic acid (2- tert-butylamino-ethyl)-methyl-amide | A9 | 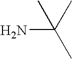 | 425.1 |
| 41 | (3SR,3aSR)-8-Fluoro-3-phenyl-3a,4-dihydro-3H- chromeno[4,3-c]pyrazole-2-carboxylic acid (2- isobutylamino-ethyl)-methyl-amide | A9 | 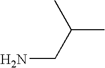 | 425.0 |
| 42 | (3SR,3aSR)-8-Fluoro-3-phenyl-3a,4-dihydro-3H- chromeno[4,3-c]pyrazole-2-carboxylic acid (3- isobutylamino-propyl)-methyl-amide | A10 | 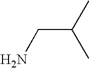 | 439.2 |
| 43 | (3SR,3aSR)-8-Fluoro-3-phenyl-3a,4-dihydro-3H- chromeno[4,3-c]pyrazole-2-carboxylic acid (3- tert-butylamino-propyl)-methyl-amide | A10 | 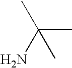 | 439.1 |
| 44 | (3SR,3aSR)-8-Fluoro-3-phenyl-3a,4-dihydro-3H- chromeno[4,3-c]pyrazole-2-carboxylic acid (2- cyclobutylamino-ethyl)-methyl-amide | A9 | 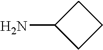 | 423.1 |
| 45 | (3SR,3aSR)-8-Fluoro-3-phenyl-3a,4-dihydro-3H- chromeno[4,3-c]pyrazole-2-carboxylic acid (2- diethylamino-ethyl)-methyl-amide | A9 | 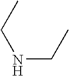 | 425.1 |
| 46 | (3SR,3aSR)-8-Fluoro-3-phenyl-3a,4-dihydro-3H- chromeno[4,3-c]pyrazole-2-carboxylic acid (3- diethylamino-propyl)-methyl-amide | A10 | 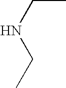 | 439.1 |
| 47 | (3SR,3aSR)-8-Fluoro-3-phenyl-3a,4-dihydro-3H- chromeno[4,3-c]pyrazole-2-carboxylic acid [2-(2- methoxy-ethylamino)-ethyl]-methyl-amide | A9 | 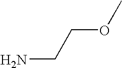 | 427.1 |
| 48 | (3SR,3aSR)-8-Fluoro-3-phenyl-3a,4-dihydro-3H- chromeno[4,3-c]pyrazole-2-carboxylic acid [3-(2- methoxy-ethylamino)-propyl]-methyl-amide | A10 | 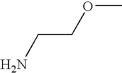 | 441.1 |
| 49 | (3SR,3aSR)-8-Fluoro-3-phenyl-3a,4-dihydro-3H- chromeno[4,3-c]pyrazole-2-carboxylic acid {2- [(2-hydroxy-ethyl)-methyl-amino]-ethyl}-methyl- amide | A9 | 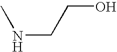 | 427.1 |
| 50 | (3SR,3aSR)-8-Fluoro-3-phenyl-3a,4-dihydro-3H- chromeno[4,3-c]pyrazole-2-carboxylic acid {3- [(2-hydroxy-ethyl)-methyl-amino]-propyl}-methyl- amide | A10 | 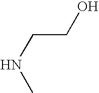 | 441.1 |
| 51 | (3SR,3aSR)-8-Fluoro-3-phenyl-3a,4-dihydro-3H- chromeno[4,3-c]pyrazole-2-carboxylic acid (2- allylamino-ethyl)-methyl-amide | A9 | 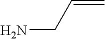 | 409.1 |
| 52 | (3SR,3aSR)-8-Fluoro-3-phenyl-3a,4-dihydro-3H- chromeno[4,3-c]pyrazole-2-carboxylic acid (3- allylamino-propyl)-methyl-amide | A10 | 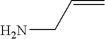 | 423.1 |
| 53 | (3SR,3aSR)-8-Fluoro-3-phenyl-3a,4-dihydro-3H- chromeno[4,3-c]pyrazole-2-carboxylic acid [2- (ethyl-methyl-amino)-ethyl]-methyl-amide | A9 | 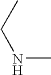 | 411.1 |
| 54 | (3SR,3aSR)-8-Fluoro-3-phenyl-3a,4-dihydro-3H- chromeno[4,3-c]pyrazole-2-carboxylic acid [3- (cyclopropylmethyl-amino)-propyl]-methyl-amide, salt with hydrochloric acid | A10 | 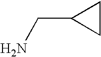 | 473.1 |
| 55 | (3SR,3aSR)-8-Fluoro-3-phenyl-3a,4-dihydro-3H- chromeno[4,3-c]pyrazole-2-carboxylic acid [3- (ethyl-methyl-amino)-propyl]-methyl-amide | A10 | 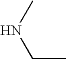 | 425.2 |
| 56 | (3SR,3aSR)-8-Fluoro-3-phenyl-3a,4-dihydro-3H- chromeno[4,3-c]pyrazole-2-carboxylic acid {3- [ethyl-(2-methoxy-ethyl)-amino]-propyl}-methyl- amide | A10 | 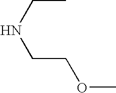 | 469.1 |
| 57 | (3SR,3aSR)-8-Fluoro-3-phenyl-3a,4-dihydro-3H- chromeno[4,3-c]pyrazole-2-carboxylic acid {2- [ethyl-(2-methoxy-ethyl)-amino]-ethyl}-methyl- amide | A9 | 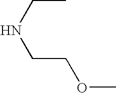 | 455.1 |
| 58 | (3RS,3aRS)-8-Fluoro-3-phenyl-3a,4-dihydro-3H- chromeno[4,3-c]pyrazole-2-carboxylic acid {2- [ethyl-(2-hydroxy-ethyl)-amino]-ethyl}-methyl- amide | A9 | 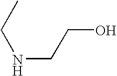 | 441.1 |
| 59 | (3RS,3aRS)-8-Fluoro-3-phenyl-3a,4-dihydro-3H- chromeno[4,3-c]pyrazole-2-carboxylic acid {3- [ethyl-(2-hydroxy-ethyl)-amino]-propyl}-methyl- amide | A10 | 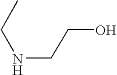 | 455.1 |
| 60 | (3SR,3aSR)-8-Fluoro-3-phenyl-3a,4-dihydro-3H- chromeno[4,3-c]pyrazole-2-carboxylic acid (2- cyclopropylamino-ethyl)-methyl-amide | A9 | 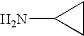 | 409.0 |
| 61 | (3SR,3aSR)-8-Fluoro-3-phenyl-3a,4-dihydro-3H- chromeno[4,3-c]pyrazole-2-carboxylic acid [2-(2- fluoro-ethylamino)-ethyl]-methyl-amide | A9 | 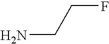 | 415.0 |
| 62 | (3SR,3aSR)-8-Fluoro-3-phenyl-3a,4-dihydro-3H- chromeno[4,3-c]pyrazole-2-carboxylic acid (2- ethylamino-ethyl)-methyl-amide | A9 | 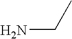 | 397.0 |
| 63 | (3SR,3aSR)-8-Fluoro-3-phenyl-3a,4-dihydro-3H- chromeno[4,3-c]pyrazole-2-carboxylic acid [2- (allyl-methyl-amino)-ethyl]-methyl-amide | A9 | 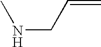 | 423.0 |
| 64 | (3SR,3aSR)-8-Fluoro-3-phenyl-3a,4-dihydro-3H- chromeno[4,3-c]pyrazole-2-carboxylic acid methyl-[2-(methyl-prop-2-ynyl-amino)-ethyl]- amide | A9 | 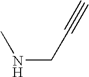 | 421.0 |
| 65 | (3SR,3aSR)-8-Fluoro-3-phenyl-3a,4-dihydro-3H- chromeno[4,3-c]pyrazole-2-carboxylic acid methyl-[2-(4-methyl-piperidin-1-yl)-ethyl]-amide | A9 | 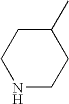 | 451.1 |
| 66 | (3SR,3aSR)-8-Fluoro-3-phenyl-3a,4-dihydro-3H- chromeno[4,3-c]pyrazole-2-carboxylic acid methyl-[3-(4-methyl-piperidin-1-yl)-propyl]-amide | A10 | 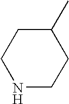 | 465.1 |

General Procedure for the Preparation of the Following Salts:

A mixture of 100 mg of the free base and 1 equivalent of the corresponding acid is dissolved in 1 ml ethanol. The solvent is removed under reduced pressure. The residue is dissolved in a small amount of dichloromethane. Diethylether is added and the resulting precipitate is filtered and dried.

If the free base as reactant is described above in the form of a salt, the free base is prepared first: The free base is e.g. obtained directly by the described reaction omitting the last step of salt preparation, or it may alternatively be obtained starting from the salt.

| | | melting |
|---|---|---|
| | | point |
| Name of the salt | corresponding acid | (° C.) |
| (3S,3aS)-8-Fluoro-3-(3-hydroxy-phenyl)-3a,4-dihydro-3H- | citric acid | 185 |
| chromeno[4,3-c]pyrazole-2-carboxylic acid methyl-(1-methyl-piperidin- | | |
| 4-yl)-amide, salt with citric acid | | |
| (3S,3aS)-8-Fluoro-3-(3-hydroxy-phenyl)-3a,4-dihydro-3H- | (+)-tartaric acid | 228 |
| chromeno[4,3-c]pyrazole-2-carboxylic acid methyl-(1-methyl-piperidin- | | |
| 4-yl)-amide, salt with (+)-tartaric acid | | |
| (3S,3aS)-8-Fluoro-3-(3-hydroxy-phenyl)-3a,4-dihydro-3H- | phosphoric acid | 191 |
| chromeno[4,3-c]pyrazole-2-carboxylic acid methyl-(1-methyl-piperidin- | | |
| 4-yl)-amide, salt with phosphoric acid | | |
| (3S,3aS)-8-Fluoro-3-(3-hydroxy-phenyl)-3a,4-dihydro-3H- | (+)-O,O′-dibenzoyl- | 201 |
| chromeno[4,3-c]pyrazole-2-carboxylic acid methyl-(1-methyl-piperidin- | D-tartaric acid | |
| 4-yl)-amide, salt with (+)-O,O′-dibenzoyl-D-tartaric acid | | |
| (3S,3aS)-8-Fluoro-3-(3-hydroxy-phenyl)-3a,4-dihydro-3H- | sulfuric acid | 181 |
| chromeno[4,3-c]pyrazole-2-carboxylic acid methyl-(1-methyl-piperidin- | | |
| 4-yl)-amide, salt with sulfuric acid | | |
| (3S,3aS)-8-Fluoro-3-phenyl-3a,4-dihydro-3H-chromeno[4,3- | citric acid | 151 |
| c]pyrazole-2-carboxylic acid methyl-(1-methyl-piperidin-4-yl)-amide, | | |
| salt with citric acid | | |
| (3S,3aS)-8-Fluoro-3-phenyl-3a,4-dihydro-3H-chromeno[4,3- | (+)-tartaric acid | 190 |
| c]pyrazole-2-carboxylic acid methyl-(1-methyl-piperidin-4-yl)-amide, | | |
| salt with (+)-tartaric acid | | |
| (3S,3aS)-8-Fluoro-3-phenyl-3a,4-dihydro-3H-chromeno[4,3- | hydrochloric acid | 213 |
| c]pyrazole-2-carboxylic acid methyl-(1-methyl-piperidin-4-yl)-amide, | | |
| salt with hydrochloric acid | | |
| (3S,3aS)-8-Fluoro-3-phenyl-3a,4-dihydro-3H-chromeno[4,3- | phosphoric acid | 200 |
| c]pyrazole-2-carboxylic acid methyl-(1-methyl-piperidin-4-yl)-amide, | | |
| salt with phosphoric acid | | |
| (3S,3aS)-8-Fluoro-3-phenyl-3a,4-dihydro-3H-chromeno[4,3- | (+)-O,O′-dibenzoyl- | 152 |
| c]pyrazole-2-carboxylic acid methyl-(1-methyl-piperidin-4-yl)-amide, | D-tartaric acid | |
| salt with (+)-O,O′-dibenzoyl-D-tartaric acid | | |
| (3S,3aS)-8-Fluoro-3-phenyl-3a,4-dihydro-3H-chromeno[4,3- | sulfuric acid | 199 |
| c]pyrazole-2-carboxylic acid methyl-(1-methyl-piperidin-4-yl)-amide, | | |
| salt with sulfuric acid | | |
| (3S,3aS)-8-Fluoro-3-phenyl-3a,4-dihydro-3H-chromeno[4,3- | citric acid | 186 |
| c]pyrazole-2-carboxylic acid methyl-piperidin-4-yl-amide, salt with | | |
| citric acid | | |
| (3S,3aS)-8-Fluoro-3-phenyl-3a,4-dihydro-3H-chromeno[4,3- | (+)-tartaric acid | 181 |
| c]pyrazole-2-carboxylic acid methyl-piperidin-4-yl-amide, salt with (+)- | | |
| tartaric acid | | |
| (3S,3aS)-8-Fluoro-3-phenyl-3a,4-dihydro-3H-chromeno[4,3- | hydrochloric acid | 71 |
| c]pyrazole-2-carboxylic acid methyl-piperidin-4-yl-amide, salt with | | |
| hydrochloric acid | | |
| (3S,3aS)-8-Fluoro-3-phenyl-3a,4-dihydro-3H-chromeno[4,3- | phosphoric acid | 175 |
| c]pyrazole-2-carboxylic acid methyl-piperidin-4-yl-amide, salt with | | |
| phosphoric acid | | |
| (3S,3aS)-8-Fluoro-3-phenyl-3a,4-dihydro-3H-chromeno[4,3- | (+)-O,O′-dibenzoyl- | 783 |
| c]pyrazole-2-carboxylic acid methyl-piperidin-4-yl-amide, salt with (+)- | D-tartaric acid | |
| O,O′-dibenzoyl-D-tartaric acid | | |
| (3S,3aS)-8-Fluoro-3-phenyl-3a,4-dihydro-3H-chromeno[4,3- | sulfuric acid | 152 |
| c]pyrazole-2-carboxylic acid methyl-piperidin-4-yl-amide, salt with | | |
| sulfuric acid | | |

Starting Materials:

### A1. (3RS,3aSR)-3-(3-Benzyloxy-phenyl)-8-fluoro-3a,4-dihydro-3H-chromeno[4,3-c]pyrazole-2-carboxylic acid dimethylamide

To 4.7 ml of an ice cold solution of phosgene in toluene (20%) is added slowly a suspension of 1.1 g (3RS,3aSR)-3-(3-Benzyloxy-phenyl)-8-fluoro-2,3,3a,4-tetrahydro-chromeno[4,3-c]pyrazole (trans diastereomer) in 10 ml tetrahydrofuran. 420 μl triethyl amine are added. The mixture is allowed to warm up to room temperature and poured slowly into 10 ml of an aqueous solution (40%) of dimethyl amine. The resulting solution is stirred at room temperature over night. The solvents are removed and the residue is dissolved in a mixture of water and ethyl acetate. The organic layer is washed with brine and dried with sodium sulfate. After evaporation of the solvent, the residue is purified by column chromatography (silica gel, heptane/ethyl acetate 4:1). The target compound is obtained as a yellowish oil.

### A2. (3RS,3aRS)-3-(3-Benzyloxy-phenyl)-8-fluoro-3a,4-dihydro-3H-chromeno[4,3-c]pyrazole-2-carboxylic acid dimethylamide

To a solution of 1.14 g (3.90 mmol) triphosgene in 5 ml toluene is added a suspension of 1.44 g (3.9 mmol) (3RS,3aRS)-3-(3-Benzyloxy-phenyl)-8-fluoro-2,3,3a,4-tetrahydro-chromeno[4,3-c]pyrazole (cis diastereomer) in 15 ml tetrahydrofuran. The mixture is cooled to 0° C. and 3.5 ml (25 mmol) triethylamine are added. The solution is allowed to warm up and stirred at room temperature over night. The reaction mixture is added dropwise to 75 ml of an icecold solution (40%) of dimethylamine in tetrahydrofuran. The mixture is warmed up to room temperature, stirred over night and the solvents are removed a reduced pressure. Water and ethyl acetate are added and the aqueous layer is extracted with ethyl acetate. The organic layer is washed with an aqueous solution of sodium bicarbonate and with brine and dried with magnesium sulfate. The solvents are removed at reduced pressure. After column chromatography (silica gel, toluene/ethyl acetate 9:1) 950 mg (3RS,3aRS)-3-(3-Benzyloxy-phenyl)-8-fluoro-3a,4-dihydro-3H-chromeno[4,3-c]pyrazole-2-carboxylic acid dimethylamide are obtained (m/z (MH⁺)=446.2).

### A3. (3SR,3aSR)-3-(3-Benzyloxy-phenyl)-8-Fluoro-3a,4-dihydro-3H-chromeno[4,3-c]pyrazole-2-carboxylic acid methyl-(1-methyl-piperidin-4-yl)-amide

The title compound is prepared analogously to the protocol described for the above for the preparation of (3RS,3aRS)-3-(3-Benzyloxy-phenyl)-8-fluoro-3a,4-dihydro-3H-chromeno[4,3-c]pyrazole-2-carboxylic acid dimethylamide (compound A2). In this case 1-methyl-4-(methylamino)-piperidine is used instead of dimethyl amine. The target molecule is purified by column chromatography (silica gel; ethyl acetate/methanol 10:1) (m/z (MH⁺)=529.6).

### A4. (3RS,3aRS)-3-(3-Benzyloxy-phenyl)-8-Fluoro-3a,4-dihydro-3H-chromeno[4,3-c]pyrazole-2-carboxylic acid (2-dimethylamino-ethyl)-methyl-amide

The title compound is prepared analogously to the protocol described for the above for the preparation of (3RS,3aRS)-3-(3-Benzyloxy-phenyl)-8-fluoro-3a,4-dihydro-3H-chromeno[4,3-c]pyrazole-2-carboxylic acid dimethylamide (compound A2). In this case N,N,N′-trimethylethylene diamine is used instead of dimethyl amine. The crude product is used without further purification (m/z (MH⁺)=489.1).

### A5. (3RS,3aRS)-3-(3-Benzyloxy-phenyl)-8-Fluoro-3a,4-dihydro-3H-chromeno[4,3-c]pyrazole-2-carboxylic acid cyclopropyl-(1-methyl-piperidin-4-yl)-amide

To a solution of 402 mg (920 μmol) (3RS,3aRS)-3-(3-Benzyloxy-phenyl)-8-fluoro-3-phenyl-3a,4-dihydro-3H-chromeno[4,3-c]pyrazole-2-carbonyl chloride (compound A8) in 25 ml dichloromethane is added at 0° C. a solution of 156 mg (1.01 mmol) 4-cycloproylamino-1-methyl piperidine. The mixture is stirred at room temperature for 2 h. The mixture is washed with an aqueous solution of hydrochloric acid (0.05 mol/l) and with brine. The organic layer is dried with sodium sulfate. The solvents are removed at reduced pressure and the residue is purified by column chromatography (silica gel; ethyl acetate/methanol/ammonia 10:0.3:0.2). 293 mg of the target compound are obtained as a colourless solid (m/z (MH⁺)=555.2).

### A6. (3RS,3aRS)-3-(3-Benzyloxy-phenyl)-8-Fluoro-3a,4-dihydro-3H-chromeno[4,3-c]pyrazole-2-carboxylic acid (3-dimethylamino-propyl)-methyl-amide, salt with hydrochloric acid

The title compound is prepared following the protocol described above for the synthesis of (3RS,3aRS)-3-(3-Benzyloxy-phenyl)-8-Fluoro-3a,4-dihydro-3H-chromeno[4,3-c]pyrazole-2-carboxylic acid cyclopropyl-(1-methyl-piperidin-4-yl)-amide (compound A5). In this case, N,N,N′-trimethyl-1,3-propandiamine is used instead of 4-cycloproylamino-1-methyl piperidine. The purified free base is dissolved in dioxane and a solution of hydrochloric acid in dioxane is added. The solvent is removed and the residue is crystallized in diisopropyl ether. The target compound is obtained as a colourless foam (m/z (MH⁺)=517.2).

### A7. (3RS,3aRS)-8-Fluoro-3-phenyl-3a,4-dihydro-3H-chromeno[4,3-c]pyrazole-2-carbonyl chloride

712 mg (7.04 mmol) triethylamine and 3.52 ml of a solution of phosgene in toluene (2 mol/l) are added to 20 ml ice-cooled toluene. The mixture is stirred for 30 min. A suspension of 1.80 g crude (3RS,3aRS)-6-Fluoro-3-phenyl-2,3,3a,4-tetrahydro-chromeno[4,3-c]pyrazole (cis diastereomer) in 5 ml toluene is added in small portions. The mixture is stirred at 0° C. for 2 h and is allowed to warm up to room temperature. The solvent is removed at reduced pressure. After chromatography (silica gel, light petroleum benzene, ethyl acetate 9:1), 470 mg (3RS,3aRS)-8-Fluoro-3-phenyl-3a,4-dihydro-3H-chromeno[4,3-c]pyrazole-2-carbonyl chloride are obtained as a pale yellow solid (m/z (MH⁺)=331.0).

### A7a. (3R,3aR)-8-Fluoro-3-phenyl-3a,4-dihydro-3H-chromeno[4,3-c]pyrazole-2-carbonyl chloride

The title compound is prepared by separation of the racemic intermediate A7 using a preparative HPLC system equipped with a chiral stationary phase. Chromatographic details are given below. The title compound is eluted as the first enantiomer and is —under the shown conditions—coeluted with traces of the corresponding racemic trans-diastereomer (which was already contained in the used racemic mixture). MS: m/z (MH⁺)=330.9

Column: CHIRALPAK IA 20 μm-180×110 mm

Mobile phase: 72% n-heptane/28% dichloromethane

Flow rate: 570 ml/min

Detection: UV 310 nm

Temperature: 25° C.

### A7b. (3S,3aS)-8-Fluoro-3-phenyl-3a,4-dihydro-3H-chromeno[4,3-c]pyrazole-2-carbonyl chloride

The title compound is prepared by separation of the racemic intermediate A7 using a preparative HPLC system equipped with a chiral stationary phase according to the procedure described for the preparation of A7a. The title compound is eluted as the second enantiomer. MS: m/z (MH⁺)=330.9, [α]_{D}^{°}=−328.5 (c=0.4549, chloroform)

### A8. (3RS,3aRS)-3-(3-Benzyloxy-phenyl)-8-fluoro-3-phenyl-3a,4-dihydro-3H-chromeno[4,3-c]pyrazole-2-carbonyl chloride

(3RS,3aRS)-3-(3-Benzyloxy-phenyl)-8-fluoro-3-phenyl-3a,4-dihydro-3H-chromeno[4,3-c]pyrazole-2-carbonyl chloride is prepared analogously to the preparation described for the synthesis of (3RS,3aRS)-8-Fluoro-3-phenyl-3a,4-dihydro-3H-chromeno[4,3-c]pyrazole-2-carbonyl chloride (compound A7), using (3RS,3aRS)-3-(3-Benzyloxy-phenyl)-8-fluoro-2,3,3a,4-tetrahydro-chromeno[4,3-c]pyrazole (cis diastereomer) instead of (3RS,3aRS)-6-Fluoro-3-phenyl-2,3,3a,4-tetrahydro-chromeno[4,3-c]pyrazole (cis diastereomer).

### A8a. (3R,3aR)-3-(3-Benzyloxy-phenyl)-8-fluoro-3a,4-dihydro-3H-chromeno[4,3-c]pyrazole-2-carbonyl chloride

The title compound is prepared by separation of the racemic intermediate A8 using a preparative HPLC system equipped with a chiral stationary phase. Chromatographic details are given below. The title compound is eluted as the first enantiomer and is —under the shown conditions—coeluted with traces of the corresponding racemic trans-diastereomer (which was already contained in the used racemic mixture). MS: m/z (MH⁺)=436.9

Column: CHIRALPAK IA 20 μm-180×110 mm

Mobile phase: 75% n-heptane/25% dichloromethane

Flow rate: 570 ml/min

Detection: UV 310 nm

Temperature: 24° C.

### A8b. (3S,3aS)-3-(3-Benzyloxy-phenyl)-8-fluoro-3a,4-dihydro-3H-chromeno[4,3-c]pyrazole-2-carbonyl chloride

The title compound is prepared by separation of the racemic intermediate A8 using a preparative HPLC system equipped with a chiral stationary phase according to the procedure described for the preparation of A8a. The title compound is eluted as the second enantiomer. MS: m/z (MH⁺)=436.9, [α]_{D}²⁰=−228.0 (c=0.4523, chloroform)

### A9. (3RS,3aRS)-8-Fluoro-3-phenyl-3a,4-dihydro-3H-chromeno[4,3-c]pyrazole-2-carboxylic acid (2-chloro-ethyl)-methyl-amide

To an ice cold solution of 636 mg (1.92 mmol) (3RS,3aRS)-8-Fluoro-3-phenyl-3a,4-dihydro-3H-chromeno[4,3-c]pyrazole-2-carbonyl chloride (compound A7) in 40 ml dichloromethane are added 389 mg (3.85 mmol) triethyl amine and after 30 min a solution of 250 mg (1.92 mmol) 2-methylaminoethylchloride hydrochloride in 7 ml methanol. The mixture is stirred for 1 h at room temperature. Additional dichloromethane and a diluted aqueous solution of hydrochloric acid are added. The organic layer is dried with magnesium sulfate. The solvents are removed at reduced pressure. The crude product is purified by column chromatography (silica gel, light petroleum/ethyl acetate 2:1). 515 mg of the title compound are obtained as a yellowish solid. (m/z (MH⁺)=388.1).

### A10. (3RS,3aRS)-8-Fluoro-3-phenyl-3a,4-dihydro-3H-chromeno[4,3-c]pyrazole-2-carboxylic acid (3-chloro-propyl)-methyl-amide

The title compound is prepared analogously to the procedure described for the synthesis of (3RS,3aRS)-8-Fluoro-3-phenyl-3a,4-dihydro-3H-chromeno[4,3-c]pyrazole-2-carboxylic acid (2-chloro-ethyl)-methyl-amide (compound A9). In this case, N-methyl-3-chloro propyl amine hydrochloride is used instead of 2-methylaminoethylchloride hydrochloride. (m/z (MH⁺)=402.0).

### A11. (3RS,3aRS)-3-(3-Benzyloxy-phenyl)-8-fluoro-3a,4-dihydro-3H-chromeno[4,3-c]pyrazole-2-carboxylic acid (2-chloro-ethyl)-methyl-amide

The title compound may be prepared similarly to compound A9 starting from compound A8.

### A12. (3RS,3a RS)-3-(3-Benzyloxy-phenyl)-8-fluoro-3a,4-dihydro-3H-chromeno[4,3-c]pyrazole-2-carboxylic acid (3-chloro-propyl)-methyl-amide

The title compound may be prepared similarly to compound A10 starting from compound A8.

### B1. (3RS,3aRS)-6-Fluoro-3-phenyl-2,3,3a,4-tetrahydro-chromeno[4,3-c]pyrazole (cis diastereomer) and (3RS,3aSR)-6-Fluoro-3-phenyl-2,3,3a,4-tetrahydro-chromeno[4,3-c]pyrazole (trans diastereomer)

To a solution of 2.00 g (7.87 mmol) 3-Benzylidene-6-fluoro-chroman-4-one (compound C1) in 40 ml ethanol are added 590 mg (11.8 mmol) hydrazine hydrate. The solution is heated to reflux over night. The mixture is cooled to 30° C. The trans diastereomer (3RS,3aSR)-6-Fluoro-3-phenyl-2,3,3a,4-tetrahydro-chromeno[4,3-c]pyrazole is filtered off and used without further purification. An aqueous solution of sodium thiosulfate and ethyl acetate are added to the filtrate. The organic layer is washed with water and dried with sodium sulfate. The solvents are removed at reduced pressure. The resulting (3RS,3aRS)-6-Fluoro-3-phenyl-2,3,3a,4-tetrahydro-chromeno[4,3-c]pyrazole (cis diastereomer) is moderately stable and used without further purification.

### B2. (3RS,3aRS)-3-(3-Benzyloxy-phenyl)-8-fluoro-2,3,3a,4-tetrahydro-chromeno[4,3-c]pyrazole (cis diastereomer) and (3RS,3aSR)-3-(3-Benzyloxy-phenyl)-8-fluoro-2,3,3a,4-tetrahydro-chromeno[4,3-c]pyrazole (trans diastereomer)

(3RS,3aRS)-3-(3-Benzyloxy-phenyl)-8-fluoro-2,3,3a,4-tetrahydro-chromeno[4,3-c]pyrazole and (3RS,3aSR)-3-(3-Benzyloxy-phenyl)-8-fluoro-2,3,3a,4-tetrahydro-chromeno[4,3-c]pyrazole are prepared analogously to the procedure described for the synthesis of (3RS,3aRS)-6-Fluoro-3-phenyl-2,3,3a,4-tetrahydro-chromeno[4,3-c]pyrazole and (3RS,3aSR)-6-Fluoro-3-phenyl-2,3,3a,4-tetrahydro-chromeno[4,3-c]pyrazole, using 3-(3-Benzyloxy-benzylidene)-6-fluoro-chroman-4-one (compound C2) instead of 3-Benzylidene-6-fluoro-chroman-4-one (compound C1).

### C1. 3-Benzylidene-6-fluoro-chroman-4-one

3-Benzylidene-6-fluoro-chroman-4-one can be prepared analogously to the procedure described by F. Fournier et al., Eur. J. Med. Chem. 1981, 16, 48-58. In this case, 6-Fluorochroman-4-one (compound D1) is used instead of chroman-4-one (m/z (MH⁺)=254.1).

### C2. 3-(3-Benzyloxy-benzylidene)-6-fluoro-chroman-4-one

3-(3-Benzyloxy-benzylidene)-6-fluoro-chroman-4-one can be prepared analogously to the procedure described for compound C1 using 3-benzyloxy-benzaldehyde instead of benzaldehyde.

### D1. 6-Fluorochroman-4-one

6-Fluorochroman-4-one is commercially available (e.g. Aldrich).

Commercial Utility

The compounds of formula I, I* and I**, and their pharmacologically and/or pharmaceutically acceptable salts (=the compounds according to the present invention) have valuable pharmacological and/or pharmaceutical properties which can make them commercially applicable. Thus, for example, the compounds according to this invention can act as inhibitors of the mitotic kinesin Eg5 and these compounds are expected to be commercially applicable in the therapy of diseases responsive to the inhibition of this kinesin, such as e.g. those diseases mentioned below. Also, for example, the compounds according to this invention can display cell-cycle dependent, anti-proliferative and/or apoptosis inducing activity.

The mitotic kinesin Eg5 is an enzyme essential for the assembly and function of the bipolar mitotic spindle. Eg5 plays essential roles during all phases of mitosis. Drugs that perturb mitosis have proven clinically effective in the treatment of many cancers. Despite the diverse array of essential spindle proteins that could be exploited as targets for the discovery of novel cancer therapies, all spindle-targeted therapeutics in clinical use today act on only one protein, tubulin. Surprisingly, kinesin Eg5 expression is most abundant in proliferating human tissues, whereas it is absent from most postmitotic cells, such as e.g. human central nervous system neurons, consistent with an exclusive or almost confined role for Eg5 in cell proliferation. In contrary to drugs that directly interfere with microtubule dynamic instability, Eg5 kinesin inhibitors are expected not to disrupt microtubule-based cellular processes, e.g. neuronal transport, that are unrelated to proliferation. During mitosis, Eg5 is essentially involved in organizing microtubules into a bipolar structure that forms the mitotic spindle. Experimental perturbation of Eg5 function causes a characteristic malformation or dysfunction of the mitotic spindle, frequently resulting in cell cycle arrest and cell death.

The compounds according to this invention can be used to modulate mitotic spindle formation, thus causing prolonged cell cycle arrest in mitosis, which is frequently followed by apoptosis. By “modulate” herein is meant altering mitotic spindle formation, including increasing and decreasing spindle formation. By “mitotic spindle formation” herein is meant organization of microtubules into bipolar structures by mitotic kinesins. By “dysfunction of the mitotic spindle” herein is meant mitotic arrest and monopolar spindle formation. “Malformation of the mitotic spindle” encompasses the splaying of mitotic spindle poles, or otherwise causing morphological perturbation of the mitotic spindle.

Further on, these compounds can be useful in the treatment of benign or malignant neoplasia. A “neoplasia” is defined by cells displaying aberrant cell proliferation and/or survival and/or a block in differentiation. A “benign neoplasia” is described by hyperproliferation of cells, incapable of forming an aggressive, metastasizing tumor in-vivo. In contrast, a “malignant neoplasia” is described by cells with multiple cellular and biochemical abnormalities, capable of forming a systemic disease, for example forming tumor metastasis in distant organs.

Various diseases are caused by aberrant cell proliferation (“hyperproliferation”) as well as evasion from apoptosis. These diseases include e.g. benign hyperplasia like that of the prostate (“BPH”) or colon epithelium, psoriasis, glomerulonephritis or osteoarthritis. Most importantly these diseases include malignant neoplasia commonly described as cancer and characterized by tumor cells finally metastasizing into distinct organs or tissues. Malignant neoplasia include solid and hematological tumors. Solid tumors are exemplified by tumors of the breast, bladder, bone, brain, central and peripheral nervous system, colon, endocrine glands (eg thyroid and adrenal cortex), esophagus, endometrium, germ cells, head and neck, kidney, liver, lung, larynx and hypopharynx, mesothelioma, sarcoma, ovary, pancreas, prostate, rectum, renal, small intestine, soft tissue, testis, stomach, skin, ureter, vagina and vulva. Malignant neoplasia include inherited cancers exemplified by retinoblastoma and Wilms tumor. In addition, malignant neoplasia include primary tumors in said organs and corresponding secondary tumors in distant organs (“tumor metastases”). Hematological tumors are exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, cancers of unknown primary site as well as AIDS related malignancies.

The invention therefore relates to a use of the compounds according to the invention in the manufacture of pharmaceutical compositions, a method of treatment or a combination according to the invention, in which the cancer to be treated is selected from the group consisting of cancer of the breast, bladder, bone, brain, central and peripheral nervous system, colon, endocrine glands, esophagus, endometrium, germ cells, head and neck, kidney, liver, lung, larynx and hypopharynx, mesothelioma, sarcoma, ovary, pancreas, prostate, rectum, renal, small intestine, soft tissue, testis, stomach, skin, ureter, vagina and vulva; inherited cancers, retinomblastoma and Wilms tumor; leukemia, lymphoma, non-Hodgkins disease, chronic and acute myeloid leukemia, acute lymphoblastic leukemia, Hodgkins disease, multiple myeloma and T-cell lymphoma; myelodysplastic syndrome, plasma cell neoplasia, paraneoplastic syndromes, cancers of unknown primary site and AIDS related malignancies.

It is to be noted that a cancer disease as well as a malignant neoplasia does not necessarily require the formation of metastases in distant organs. Certain tumors exert devastating effects on the primary organ itself through their aggressive growth properties. These can lead to the destruction of the tissue and organ structure finally resulting in failure of the assigned organ function.

Neoplastic cell proliferation might affect normal cell behaviour and organ function. For example the formation of new blood vessels, a process described as neovascularization, is induced by tumors or tumor metastases. Compounds according to this invention can be commercially applicable for the treatment of pathophysiological relevant processes caused by benign or neoplastic cell proliferation, such as but not limited to neovascularization by unphysiological proliferation of vascular endothelial cells.

Drug resistance is of particular importance for the frequent failure of standard cancer therapeutics. This drug resistance is caused by various cellular and molecular mechanisms like overexpression of drug efflux pumps or mutation within the cellular target protein. The commercial applicability of the compounds according to this invention is not limited to 1^{st} line treatment of patients. Patients with resistance to defined cancer chemotherapeutics or target specific anti-cancer drugs (2^{nd} or 3^{rd} line treatment) can be also amenable for treatment with the compounds according to this invention.

Due to their cellular anti-proliferative properties, compounds according to the present invention may be also commercially usable for treatment of diseases associated with cell cycle and cell proliferation, such as, besides cancer discussed above, for example, fibroproliferative and differentiative disorders, psoriasis, rheumatoid arthritis, atherosclerosis, hyperplasia, restenosis, cardiac hypertrophy, (auto)immune disorders, fungal disorders, bone diseases, or acute or chronic inflammation. Thus, the invention relates to compounds according to the invention for use in the treatment of diseases.

Compounds according to the present invention can be commercially applicable for treatment, prevention or amelioration of the diseases of benign and malignant behavior as described before, such as e.g. benign or malignant neoplasia, particularly cancer (such as e.g. any of those cancer diseases described above), especially a cancer that is susceptible to Eg5 inhibition.

In the context of their properties, functions and usabilities mentioned herein, the compounds according to the present invention are expected to be distinguished by valuable and desirable effects related therewith, such as e.g. by low toxicity, superior bioavailability in general (such as e.g. good enteral absorption), superior therapeutic window, absence of significant side effects, and/or further beneficial effects related with their therapeutic and pharmaceutical suitability.

The invention further includes a method for treating (hyper)proliferative diseases and/or disorders responsive to the induction of apoptosis, particularly those diseases, disorders, conditions or illnesses mentioned above, in mammals, including humans, suffering therefrom comprising administering to said mammals in need thereof a pharmacologically and/or pharmaceutically active and therapeutically effective and tolerable amount of one or more of the compounds according to this invention.

The present invention further includes a method useful to modulate apoptosis and/or aberrant cell growth in the therapy of benign or malignant neoplastic diseases, such as e.g. cancer, comprising administering to a subject in need of such therapy a pharmacologically and/or pharmaceutically active active and therapeutically effective and tolerable amount of one or more of the compounds according to this invention.

The invention further includes a method for modulating, particularly inhibiting, Eg5 activity in cells comprising administering a pharmacologically and/or pharmaceutically active active and therapeutically effective and tolerable amount of one or more of the compounds according to this invention to a patient in need of such modulation, particularly inhibition.

The invention further includes a method for modulating Eg5 kinesin activity comprising administering a therapeutically effective and tolerable amount of one or more compounds according to the invention to a mammal in need of said modulation.

The present invention further includes a method to modulate the mitotic spindle, i.e., for example, altering mitotic spindle formation, including decreasing spindle formation, or increasing or decreasing spindle pole separation causing malformation of the mitotic spindle poles, comprising administering a pharmacologically and/or pharmaceutically active active and therapeutically effective and tolerable amount of one or more of the compounds according to this invention to a patient in need of such modulation.

The present invention further includes a method to inhibit mitosis in cells comprising administering a pharmacologically and/or pharmaceutically active active and therapeutically effective and tolerable amount of one or more of the compounds according to this invention to a patient in need of such inhibition.

The present invention further includes a method for treating, preventing or ameliorating diseases and/or disorders associated with Eg5 kinesin activity, such as, for example, (hyper)proliferative diseases and/or disorders responsive to induction of apoptosis, for example, benign neoplasia or malignant neoplasia, e.g. cancer, in a mammal comprising administering a pharmacologically and/or pharmaceutically active active and therapeutically effective and tolerable amount of one or more compounds according to the present invention to said mammal in need thereof.

The invention further includes a method for treating, preventing or ameliorating (hyper)proliferative diseases and/or disorders responsive to induction of apoptosis, such as, for example, benign or malignant neoplasia, e.g. cancer, in a mammal comprising administering a therapeutically effective and tolerable amount of one or more compounds according to the invention to said mammal in need thereof.

The present invention further relates to the use of the compounds according to this invention for the production of pharmaceutical compositions which are employed for the treatment, prophylaxis and/or amelioration of one or more of the illnesses mentioned.

The invention further relates to the use of the compounds according to the invention in the manufacture of pharmaceutical compositions for treating (hyper)proliferative diseases and/or disorders responsive to induction of apoptosis, such as, for example, benign and/or malignant neoplasia, e.g. cancer.

The present invention further relates to the use of the compounds according to this invention for the production of pharmaceutical compositions which can be used in the treatment, prevention or amelioration of (hyper)proliferative diseases of benign or malignant behaviour and/or disorders responsive to the induction of apoptosis in a mammal, such as, for example, benign or malignant neoplasia, e.g. cancer.

The present invention further relates to the use of the compounds according to this invention for the production of pharmaceutical compositions which can be used use in the treatment, prevention or amelioration of disorders responsive to arresting of aberrant cell growth and/or induction of apoptosis.

The present invention further relates to the use of the compounds according to this invention for the production of pharmaceutical compositions for treating, preventing or ameliorating benign or malignant neoplasia, particularly cancer, such as e.g. any of those cancer diseases described above.

The present invention further relates to pharmaceutical compositions comprising one or more of the compounds according to this invention and a pharmaceutically acceptable carrier or diluent.

The present invention further relates to pharmaceutical compositions made by combining one or more of the compounds according to this invention and a pharmaceutically acceptable carrier or diluent.

The present invention further relates to pharmaceutical compositions comprising one or more of the compounds according to this invention and pharmaceutically acceptable auxiliaries and/or excipients.

The present invention also relates to pharmaceutical compositions for treating (hyper)proliferative diseases and/or disorders responsive to induction of apoptosis, which include benign neoplasia and malignant neoplasia, including cancer, comprising a compound according to this invention.

The present invention further relates to combinations comprising one or more of the compounds according to this invention and pharmaceutically acceptable auxiliaries, excipients and/or vehicles, e.g. for treating, preventing or ameliorating benign or malignant neoplasia, particularly cancer, such as e.g. any of those cancer diseases described above.

The present invention further relates to a combination comprising a compound according to this invention and a pharmaceutically acceptable excipient, carrier and/or diluent, e.g. for treating, preventing or ameliorating benign or malignant neoplasia, particularly cancer, such as e.g. any of those cancer diseases described above.

The present invention further relates to a composition consisting essentially of a therapeutically effective and tolerable amount of one or more compounds according to this invention together with the usual pharmaceutically acceptable vehicles, diluents and/or excipients for use in therapy, e.g. for treating, preventing or ameliorating hyperproliferative diseases, such as e.g. cancer, and/or disorders responsive to induction of apoptosis.

The present invention further relates to compounds according to this invention for use in therapy, such as, for example, in the treatment, prevention or amelioration of (hyper)proliferative diseases of benign or malignant behaviour and/or disorders responsive to the induction of apoptosis, such as e.g. those diseases mentioned herein, particularly cancer.

The present invention further relates to compounds according to this invention having anti-proliferative and/or apoptosis inducing activity.

The present invention further relates to compounds according to this invention having Eg5 inhibiting properties.

The present invention further relates to pharmaceutical compositions according to this invention having Eg5 inhibiting properties.

The present invention further relates to pharmaceutical compositions according to this invention having anti-proliferative activity.

The present invention further relates to pharmaceutical compositions according to this invention having apoptosis inducing activity.

The invention further relates to the use of a pharmaceutical composition comprising one or more of the compounds according to this invention as sole active ingredient(s) and a pharmaceutically acceptable carrier or diluent in the manufacture of pharmaceutical products for the treatment and/or prophylaxis of the illnesses mentioned above.

Additionally, the invention relates to an article of manufacture, which comprises packaging material and a pharmaceutical agent contained within said packaging material, wherein the pharmaceutical agent is therapeutically effective inhibiting Eg5 and/or inhibiting cellular (hyper)proliferation and/or inducing apoptosis, ameliorating the symptoms of a Eg5 mediated disease and/or a (hyper)proliferative disease and/or a disorder responsive to the induction of apoptosis, and wherein the packaging material comprises a label or package insert which indicates that the pharmaceutical agent is useful for preventing or treating a Eg5 mediated disease and/or a (hyper)proliferative disease and/or a disorder responsive to the induction of apoptosis, and wherein said pharmaceutical agent comprises one or more compounds according to the invention. The packaging material, label and package insert otherwise parallel or resemble what is generally regarded as standard packaging material, labels and package inserts for pharmaceuticals having related utilities.

The pharmaceutical compositions according to this invention are prepared by processes which are known per se and familiar to the person skilled in the art. As pharmaceutical compositions, the compounds of the invention (=active compounds) are either employed as such, or preferably in combination with suitable pharmaceutical auxiliaries and/or excipients, e.g. in the form of tablets, coated tablets, dragees, pills, cachets, granules, capsules, caplets, suppositories, patches (e.g. as TTS), emulsions (such as e.g. micro-emulsions or lipid emulsions), suspensions (such as e.g. nano suspensions), gels, solubilisates or solutions (e.g. sterile solutions), or encapsuled in liposomes or as beta-cyclodextrine inclusion complexes or the like, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries, vehicles, excipients, diluents, carriers or adjuvants which are suitable for the desired pharmaceutical formulations, preparations or compositions on account of his/her expert knowledge. In addition to solvents, gel formers, ointment bases and other active compound excipients, for example antioxidants, dispersants, emulsifiers, preservatives, solubilizers (such as e.g. polyoxyethylenglyceroltriricinoleat 35, PEG 400, Tween 80, Solutol HS15 or the like), colorants, complexing agents, permeation promoters, stabilizers, fillers, binders, thickeners, disintegrating agents, buffers, pH regulators (e.g. to obtain neutral, alkaline or acidic formulations), polymers, lubricants, coating agents, propellants, tonicity adjusting agents, surfactants, flavorings, sweeteners or dyes, can be used.

In particular, auxiliaries and/or excipients of a type appropriate to the desired formulation and the desired mode of administration are used.

The administration of the compounds, pharmaceutical compositions or combinations according to the invention may be performed in any of the generally accepted modes of administration available in the art. Illustrative examples of suitable modes of administration include intravenous, oral, nasal, parenteral, topical, transdermal and rectal delivery. Oral and intravenous delivery are preferred.

For the treatment of dermatoses, the compounds of the invention can be in particular administered in the form of those pharmaceutical compositions which are suitable for topical application. For the production of the pharmaceutical compositions, the compounds of the invention (=active compounds) are preferably mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, lotions, pastes, gels or solutions.

The pharmaceutical compositions according to the invention can be prepared by processes known per se. The dosage of the compounds of the invention (=active compounds) is carried out in the order of magnitude customary for Eg5 inhibitors, inhibitors for cellular (hyper)proliferation or apoptosis inducers. Topical application forms (such as ointments) for the treatment of dermatoses thus contain the active compounds in a concentration of, for example, 0.1-99%. The customary dose in the case of systemic therapy (p.o.) may be between 0.03 and 60 mg/kg per day, (i. v.) may be between 0.03 and 60 mg/kg/h. In another embodiment, the customary dose in the case of systemic therapy (p.o.) is between 0.3 and 30 mg/kg per day, (i. v.) is between 0.3 and 30 mg/kg/h.

The choice of the optimal dosage regime and duration of medication, particularly the optimal dose and manner of administration of the active compounds necessary in each case can be determined by a person skilled in the art on the basis of his/her expert knowledge.

Depending upon the particular disease, to be treated or prevented, additional therapeutic active agents, which are normally administered to treat or prevent that disease, may optionally be coadministered with the compounds according to this invention. As used herein, additional therapeutic agents that are normally administered to treat or prevent a particular disease are known as appropriate for the disease being treated.

For example, compounds according to this invention may be combined with one or more standard therapeutic agents used for treatment of the diseases as mentioned before.

In one particular embodiment, compounds according to this invention may be combined with one or more art-known anti-cancer agents, such as e.g. with one or more chemotherapeutic and/or target specific anti-cancer agents as described below.

Examples of known chemotherapeutic anti-cancer agents frequently used in combination therapy include, but not are limited to (i) alkylating/carbamylating agents such as Cyclophosphamid (Endoxan®), Ifosfamid (Holoxan®), Thiotepa (Thiotepa Lederle®), Melphalan (Alkeran®), or chloroethylnitrosourea (BCNU); (ii) platinum derivatives like cis-platin (Platinex® BMS), oxaliplatin, satraplatin or carboplatin (Cabroplat® BMS); (iii) antimitotic agents/tubulin inhibitors such as vinca alkaloids (vincristine, vinblastine, vinorelbine), taxanes such as Paclitaxel (Taxol®), Docetaxel (Taxotere®) and analogs as well as new formulations and conjugates thereof, epothilones such as Epothilone B (Patupilone®), Azaepothilone (Ixabepilone®) or ZK-EPO, a fully synthetic epothilone B analog; (iv) topoisomerase inhibitors such as anthracyclines (exemplified by Doxorubicin/Adriblastin®), epipodophyllotoxines (examplified by Etoposide/Etopophos®) and camptothecin and camptothecin analogs (exemplified by Irinotecan/Camptosar® or Topotecan/Hycamtin®); (v) pyrimidine antagonists such as 5-fluorouracil (5-FU), Capecitabine (Xeloda®), Arabinosylcytosine/Cytarabin (Alexan®) or Gemcitabine (Gemzar®); (vi) purin antagonists such as 6-mercaptopurine (Puri-Nethol®), 6-thioguanine or fludarabine (Fludara®) and finally (vii) folic acid antagonists such as methotrexate (Farmitrexat®) or premetrexed (Alimta®).

Examples of target specific anti-cancer drug classes used in experimental or standard cancer therapy include but are not limited to (i) kinase inhibitors such as e.g. Imatinib (Glivec®), ZD-1839/Gefitinib (Iressa®), Bay43-9006 (Sorafenib), SU11248/Sunitinib (Sutent®) or OSI-774/Erlotinib (Tarceva®); (ii) proteasome inhibitors such as PS-341/Bortezumib (Velcade®); (iii) histone deacetylase inhibitors like SAHA, PXD101, MS275, MGCD0103, Depsipeptide/FK228, NVP-LBH589, NVP-LAQ824, Valproic acid (VPA) and butyrates (iv) heat shock protein 90 inhibitors like 17-allylaminogeldanamycin (17-AAG); (v) vascular targeting agents (VTAs) like combretastin A4 phosphate or AVE8062/AC7700 and anti-angiogenic drugs like the VEGF antibodies, such as Bevacizumab (Avastin®), or KDR tyrosine kinase inhibitors such as PTK787/ZK222584 (Vatalanib); (vi) monoclonal antibodies such as Trastuzumab (Herceptin®) or Rituximab (MabThera/Rituxan®) or Alemtuzumab (Campath®) or Tositumab (Bexxar®) or C225/Cetuximab (Erbitux®) or Avastin (see above) as well as mutants and conjugates of monoclonal antibodies, e.g. Gemtuzumab ozogamicin (Mylotarg®) or Ibritumomab tiuxetan (Zevalin®), and antibody fragments; (vii) oligonucleotide based therapeutics like G-3139/Oblimersen (Genasense®); (viii) Toll-like receptor/TLR 9 agonists like Promune®, TLR 7 agonists like Imiquimod (Aldara®) or Isatoribine and analogues thereof, or TLR 7/8 agonists like Resiquimod as well as immunostimulatory RNA as TLR 7/8 agonists; (ix) protease inhibitors (x) hormonal therapeutics such as anti-estrogens (e.g. Tamoxifen or Raloxifen), anti-androgens (e.g. Flutamide or Casodex), LHRH analogs (e.g. Leuprolide, Goserelin or Triptorelin) and aromatase inhibitors.

Other known target specific anti-cancer agents which may be used for combination therapy include bleomycin, retinoids such as all-trans retinoic acid (ATRA), DNA methyltransferase inhibitors such as the 2-deoxycytidine derivative Decitabine (Dacogen®) and 5-azacytidine, alanosine, cytokines such as interleukin-2, interferons such as interferon α2 or interferon-γ, death receptor agonists, such as TRAIL, DR4/5 agonistic antibodies, FasL and TNF-R agonists.

As exemplary anti-cancer agents, which may be useful in the combination therapy according to the present invention, any of the following drugs may be mentioned, without being restricted thereto, 5 FU, actinomycin D, ABARELIX, ABCIXIMAB, ACLARUBICIN, ADAPALENE, ALEMTUZUMAB, ALTRETAMINE, AMINOGLUTETHIMIDE, AMIPRILOSE, AMRUBICIN, ANASTROZOLE, ANCITABINE, ARTEMISININ, AZATHIOPRINE, BASILIXIMAB, BENDAMUSTINE, BEVACIZUMAB, BEXXAR, BICALUTAMIDE, BLEOMYCIN, BORTEZOMIB, BROXURIDINE, BUSULFAN, CAMPATH, CAPECITABINE, CARBOPLATIN, CARBOQUONE, CARMUSTINE, CETRORELIX, CHLORAMBUCIL, CHLORMETHINE, CISPLATIN, CLADRIBINE, CLOMIFENE, CYCLOPHOSPHAMIDE, DACARBAZINE, DACLIZUMAB, DACTINOMYCIN, DAUNORUBICIN, DECITABINE, DESLORELIN, DEXRAZOXANE, DOCETAXEL, DOXIFLURIDINE, DOXORUBICIN, DROLOXIFENE, DROSTANOLONE, EDELFOSINE, EFLORNITHINE, EMITEFUR, EPIRUBICIN, EPITIOSTANOL, EPTAPLATIN, ERBITUX, ERLOTINIB, ESTRAMUSTINE, ETOPOSIDE, EXEMESTANE, FADROZOLE, FINASTERIDE, FLOXURIDINE, FLUCYTOSINE, FLUDARABINE, FLUOROURACIL, FLUTAMIDE, FORMESTANE, FOSCARNET, FOSFESTROL, FOTEMUSTINE, FULVESTRANT, GEFITINIB, GENASENSE, GEMCITABINE, GLIVEC, GOSERELIN, GUSPERIMUS, HERCEPTIN, IDARUBICIN, IDOXURIDINE, IFOSFAMIDE, IMATINIB, IMPROSULFAN, INFLIXIMAB, IRINOTECAN, IXABEPILONE, LANREOTIDE, LETROZOLE, LEUPRORELIN, LOBAPLATIN, LOMUSTINE, LUPROLIDE, MELPHALAN, MERCAPTOPURINE, METHOTREXATE, METUREDEPA, MIBOPLATIN, MIFEPRISTONE, MILTEFOSINE, MIRIMOSTIM, MITOGUAZONE, MITOLACTOL, MITOMYCIN, MITOXANTRONE, MIZORIBINE, MOTEXAFIN, MYLOTARG, NARTOGRASTIM, NEBAZUMAB, NEDAPLATIN, NILUTAMIDE, NIMUSTINE, OCTREOTIDE, ORMELOXIFENE, OXALIPLATIN, PACLITAXEL, PALIVIZUMAB, PATUPILONE, PEGASPARGASE, PEGFILGRASTIM, PEMETREXED, PENTETREOTIDE, PENTOSTATIN, PERFOSFAMIDE, PIPOSULFAN, PIRARUBICIN, PLICAMYCIN, PREDNIMUSTINE, PROCARBAZINE, PROPAGERMANIUM, PROSPIDIUM CHLORIDE, RALOXIFEN, RALTITREXED, RANIMUSTINE, RANPIRNASE, RASBURICASE, RAZOXANE, RITUXIMAB, RIFAMPICIN, RITROSULFAN, ROMURTIDE, RUBOXISTAURIN, SARGRAMOSTIM, SATRAPLATIN, SIROLIMUS, SOBUZOXANE, SORAFENIB, SPIROMUSTINE, STREPTOZOCIN, SUNITINIB, TAMOXIFEN, TASONERMIN, TEGAFUR, TEMOPORFIN, TEMOZOLOMIDE, TENIPOSIDE, TESTOLACTONE, THIOTEPA, THYMALFASIN, TIAMIPRINE, TOPOTECAN, TOREMIFENE, TRAIL, TRASTUZUMAB, TREOSULFAN, TRIAZIQUONE, TRIMETREXATE, TRIPTORELIN, TROFOSFAMIDE, UREDEPA, VALRUBICIN, VATALANIB, VERTEPORFIN, VINBLASTINE, VINCRISTINE, VINDESINE, VINORELBINE, VOROZOLE and ZEVALIN.

The anti-cancer agents mentioned herein above as combination partners of the compounds according to this invention are meant to include pharmaceutically acceptable derivatives thereof, such as e.g. their pharmaceutically acceptable salts.

The person skilled in the art is aware on the base of his/her expert knowledge of the kind, total daily dosage(s) and administration form(s) of the additional therapeutic agent(s) coadministered. Said total daily dosage(s) can vary within a wide range.

In practicing the present invention, the compounds according to this invention may be administered in combination therapy separately, sequentially, simultaneously, concurrently or chronologically staggered (such as e.g. as combined unit dosage forms, as separate unit dosage forms, as adjacent discrete unit dosage forms, as fixed or non-fixed combinations, as kit-of-parts or as admixtures) with one or more standard therapeutics (chemotherapeutic and/or target specific anti-cancer agents), in particular art-known anti-cancer agents, such as any of e.g. those mentioned above.

In this context, the present invention further relates to a combination comprising

a first active ingredient, which is at least one compound according to this invention, and

a second active ingredient, which is at least one art-known anti-cancer agent, such as e.g. one or more of those mentioned herein above,

for separate, sequential, simultaneous, concurrent or chronologically staggered use in therapy, such as e.g. in therapy of any of those diseases mentioned herein.

The term “combination” according to this invention may be present as a fixed combination, a non-fixed combination or a kit-of-parts.

A “fixed combination” is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A “kit-of-parts” is defined as a combination wherein the said first active ingredient and the said second active ingredient are present in more than one unit. One example of a “kit-of-parts” is a combination wherein the said first active ingredient and the said second active ingredient are present separately. The components of the kit-of-parts may be administered separately, sequentially, simultaneously, concurrently or chronologically staggered.

The present invention further relates to a pharmaceutical composition comprising

a first active ingredient, which is at least one compound according to this invention, and

a second active ingredient, which is at least one art-known anti-cancer agent, such as e.g. one or more of those mentioned herein above, and, optionally,

a pharmaceutically acceptable carrier or diluent,

for separate, sequential, simultaneous, concurrent or chronologically staggered use in therapy.

The present invention further relates to a combination product comprising

a.) at least one compound according to this invention formulated with a pharmaceutically acceptable carrier or diluent, and

b.) at least one art-known anti-cancer agent, such as e.g. one or more of those mentioned herein above, formulated with a pharmaceutically acceptable carrier or diluent.

The present invention further relates to a kit-of-parts comprising a preparation of a first active ingredient, which is a compound according to this invention, and a pharmaceutically acceptable carrier or diluent; a preparation of a second active ingredient, which is an art-known anti-cancer agent, such as one of those mentioned above, and a pharmaceutically acceptable carrier or diluent; for simultaneous, concurrent, sequential, separate or chronologically staggered use in therapy. Optionally, said kit comprises instructions for its use in therapy, e.g. to treat (hyper)proliferative diseases and/or disorders responsive to the induction of apoptosis, such as e.g. cancer, more precisely, any of those cancer diseases described above.

The present invention further relates to a combined preparation comprising at least one compound according to this invention and at least one art-known anti-cancer agent for simultaneous, concurrent, sequential or separate administration.

The present invention further relates to combinations, compositions, formulations, preparations or kits according to the present invention having Eg5 inhibitory activity and/or anti-proliferative and/or apoptosis inducing properties.

In addition, the present invention further relates to a method for treating in combination therapy (hyper)proliferative diseases and/or disorders responsive to the induction of apoptosis, such as e.g. cancer, in a patient comprising administering a combination, composition, formulation, preparation or kit as described herein to said patient in need thereof.

In addition, the present invention further relates to a method for treating (hyper)proliferative diseases of benign or malignant behaviour and/or disorders responsive to the induction of apoptosis, such as e.g. cancer, in a patient comprising administering in combination therapy separately, simultaneously, concurrently, sequentially or chronologically staggered a pharmaceutically active and therapeutically effective and tolerable amount of a pharmaceutical composition, which comprises a compound according to this invention and a pharmaceutically acceptable carrier or diluent, and a pharmaceutically active and therapeutically effective and tolerable amount of one or more art-known anti-cancer agents, such as e.g. one or more of those mentioned herein, to said patient in need thereof.

In further addition, the present invention relates to a method for treating, preventing or ameliorating (hyper)proliferative diseases and/or disorders responsive to induction of apoptosis, such as e.g. benign or malignant neoplasia, e.g. cancer, particularly any of those cancer diseases mentioned herein, in a patient comprising administering separately, simultaneously, concurrently, sequentially or chronologically staggered to said patient in need thereof an amount of a first active compound, which is a compound according to the present invention, and an amount of at least one second active compound, said at least one second active compound being a standard therapeutic agent, particularly at least one art-known anti-cancer agent, such as e.g. one or more of those chemotherapeutic and target-specific anti-cancer agents mentioned herein, wherein the amounts of the first active compound and said second active compound result in a therapeutic effect.

In yet further addition, the present invention relates to a method for treating, preventing or ameliorating (hyper)proliferative diseases and/or disorders responsive to induction of apoptosis, such as e.g. benign or malignant neoplasia, e.g. cancer, particularly any of those cancer diseases mentioned herein, in a patient comprising administering a combination according to the present invention.

In addition, the present invention further relates to the use of a composition, combination, formulation, preparation or kit according to this invention in the manufacture of a pharmaceutical product, such as e.g. a commercial package or a medicament, for treating, preventing or ameliorating (hyper)proliferative diseases, such as e.g. cancer, and/or disorders responsive to the induction of apoptosis, particularly those diseases mentioned herein, such as e.g. malignant or benign neoplasia.

The present invention further relates to a commercial package comprising one or more compounds of the present invention together with instructions for simultaneous, concurrent, sequential or separate use with one or more chemotherapeutic and/or target specific anti-cancer agents, such as e.g. any of those mentioned herein.

The present invention further relates to a commercial package consisting essentially of one or more compounds of the present invention as sole active ingredient together with instructions for simultaneous, concurrent, sequential or separate use with one or more chemotherapeutic and/or target specific anti-cancer agents, such as e.g. any of those mentioned herein.

The present invention further relates to a commercial package comprising one or more chemotherapeutic and/or target specific anti-cancer agents, such as e.g. any of those mentioned herein, together with instructions for simultaneous, concurrent, sequential or separate use with one or more compounds according to the present invention.

The compositions, combinations, preparations, formulations, kits or packages mentioned in the context of the combination therapy according to this invention may also include more than one of the compounds according to this invention and/or more than one of the art-known anti-cancer agents mentioned.

The first and second active ingredient of a combination or kit-of-parts according to this invention may be provided as separate formulations (i.e. independently of one another), which are subsequently brought together for simultaneous, concurrent, sequential, separate or chronologically staggered use in combination therapy; or packaged and presented together as separate components of a combination pack for simultaneous, concurrent, sequential, separate or chronologically staggered use in combination therapy.

The type of pharmaceutical formulation of the first and second active ingredient of a combination or kit- of-parts according to this invention can be similar, i.e. both ingredients are formulated in separate tablets or capsules, or can be different, i.e. suited for different administration forms, such as e.g. one active ingredient is formulated as tablet or capsule and the other is formulated for e.g. intravenous administration.

The amounts of the first and second active ingredients of the combinations, compositions or kits according to this invention may together comprise a therapeutically effective amount for the treatment, prophylaxis or amelioration of a (hyper)proliferative diseases and/or a disorder responsive to the induction of apoptosis, particularly one of those diseases mentioned herein, such as e.g. malignant or benign neoplasia, especially cancer, like any of those cancer diseases mentioned herein.

In addition, compounds according to the present invention can be used in the pre- or post-surgical treatment of cancer.

In further addition, compounds of the present invention can be used in combination with radiation therapy.

A combination according to this invention can refer to a composition comprising both the compound(s) according to this invention and the other active anti-cancer agent(s) in a fixed combination (fixed unit dosage form), or a medicament pack comprising the two or more active ingredients as discrete separate dosage forms (non-fixed combination). In case of a medicament pack comprising the two or more active ingredients, the active ingredients are preferably packed into blister cards which are suited for improving compliance.

Each blister card preferably contains the medicaments to be taken on one day of treatment. If the medicaments are to be taken at different times of day, the medicaments can be disposed in different sections on the blister card according to the different ranges of times of day at which the medicaments are to be taken (for example morning and evening or morning, midday and evening). The blister cavities for the medicaments to be taken together at a particular time of day are accommodated in the respective range of times of day. The various times of day are, of course, also put on the blister in a clearly visible way. It is also possible, of course, for example to indicate a period in which the medicaments are to be taken, for example stating the times.

The daily sections may represent one line of the blister card, and the times of day are then identified in chronological sequence in this column.

Medicaments which must be taken together at a particular time of day are placed together at the appropriate time on the blister card, preferably a narrow distance apart, allowing them to be pushed out of the blister easily, and having the effect that removal of the dosage form from the blister is not forgotten.

Biological Investigations

The ATPase activity of Eg5 kinesin motor domains (Cytoskeleton, cat. No. EG01) can be used to monitor the effects of modulating agents. The test compounds are dissolved as 10 mM solutions in dimethylsulfoxide (DMSO). 2 μl of appropriate DMSO dilutions of the test compounds are added to each well of a 96 well flat bottom plate. Each compound dilution is tested as triplicates. The reagents are added and the final reaction of the standard assay contains 15 mM Pipes, pH 6.8, 5.0 mM MgCl₂, 0.5 mM KCl, 1 mM EGTA, 0.1 mg/ml BSA, 1 μM Paclitaxel, 250 nM preformed microtubules (Cytoskeleton, cat. No. MT001), 300 μM ATP, and Eg5 protein (50 ng) in a reaction volume of 100 μl. The controls include buffer wells with ATP and 2% DMSO. Reactions are started by the addition of ATP, incubated at room temperature for 30 min., and terminated by removing 20 μl of the reaction volume and adding it to 80 μl of 1 M perchloric acid, followed by the addition of 80 μl Malachite green reagent. Malachite green reagent is prepared by mixing a solution of 4.2 g ammonium molybdate in 100 ml 4 N HCl with a solution of 0.135 g Malachite green in 300 ml H₂O. The reactions are incubated for a further 20 min. and then read at 615 nm.

The corresponding IC₅₀ values of the compounds for Eg5 inhibition are determined from the concentration-effect curves.

Representative inhibitory values [measured as −log IC₅₀ (mol/l)] determined in the aforementioned assay follow from the following table A, in which the numbers of the compounds correspond to the numbers of the examples.

TABLE A
Inhibition of Eg5 activity

| Compound | −log IC₅₀ [mol/l] |
|---|---|
| 1, 2, 2b, 3, 3b, 4 to | The inhibitory values of these listed |
| 9, 10 to 16, 17 to | compounds are all ≧6.2 |
| 20, 22 to 27, 28 to | |
| 29 | |

The anti-proliferative/cytotoxic activity of the compounds described herein can be tested on subclones of RKO human colon adenocarcinoma cells (Schmidt et al., Oncogene 19, 2423-2429; 2000) using the Alamar Blue cell viability assay (described in O'Brien et al. Eur J Biochem 267, 5421-5426, 2000). The compounds are dissolved as 10 mM solutions in DMSO and subsequently diluted in semi-logarithmic steps. DMSO dilutions are further diluted 1:100 into Dulbecco's modified Eagle's medium (DMEM) containing 10% fetal calf serum to a final concentration twice as much as the final concentration in the test. RKO subclones are seeded into 96 well flat bottom plates at a density of 4000 cells per well in a volume of 50 μl per well. 24 hours after seeding the 50 μl each of the compound dilutions in DMEM medium are added into each well of the 96 well plate. Each compound dilution is tested as triplicates. Wells containing untreated control cells are filled with 50 μl DMEM medium containing 1% DMSO. The cells are then incubated with the substances for 72 hours at 37° C. in a humidified atmosphere containing 5% carbon dioxide. To determine the viability of the cells, 10 μl of an Alamar Blue solution (Biosource) are added and the fluorescence is measured at an extinction of 544 nm and an emission of 590 nm. For the calculation of the cell viability the emission value from untreated cells is set as 100% viability and the emission rates of treated cells are set in relation to the values of untreated cells. Viabilities are expressed as % values. The Graphpad Prism program (GraphPad Software, Inc) is used for the calculation of EC₅₀ values for anti-proliferative/cytotoxic activity out of the obtained dose-response curves.

To determine the cell cycle specific mode of action, subclones of RKO colon adenocarcinoma cells (RKOp27 as described by Schmidt et al. in Oncogene 19, 2423-2429; 2000) are seeded into 96 well flat bottom plates at a density of 16000 cells per well in a volume of 50 μl per well in DMEM growth medium with 10% FCS containing 10 μM Ponasterone A. 24 hours after seeding the 50 μl each of the compound dilutions in DMEM medium are added into each well of the 96-well plate. Each compound dilution is tested as triplicates. Wells containing untreated control cells are filled with 50 μl DMEM medium containing 1% DMSO. The cells are then incubated with the substances for 72 hours at 37° C. in a humidified atmosphere containing 5% carbon dioxide. To determine the viability of the cells, 10 μl of an Alamar Blue solution (Biosource) are added and the fluorescence is measured at an extinction of 544 nm and an emission of 590 nm. For the calculation of the cell viability the emission value from untreated cells is set as 100% viability and the emission rates of treated cells are set in relation to the values of untreated cells. Viabilities are expressed as % values. The Graphpad Prism program is used for the calculation of EC₅₀ values out of the obtained dose-response curves. Viability is compared of proliferating cells grown in the absence of the inducer Ponasterone A, versus viability of cells arrested by the expression of ectopic p27Kip1 induced by Ponasterone A.

Representative values for anti-proliferation/cytotoxicity [measured as −log EC₅₀ (mol/l)] determined in the aforementioned assays follow from the following table B, in which the numbers of the compounds correspond to the numbers of the examples.

TABLE B
Anti-proliferative/cytotoxic activity on RKO colon cancer cells

| | Examples |
|---|---|
| −log EC₅₀ [mol/l] | 2b, 3, 3b, 4, 6, 9, 9a, 10, 11, 15, 16, 16a, 18, 18a, |
| RKO p27 uninduced | 19, 22, 24, 25, 26, 27, 27a, 34, 37, 38, 39, 40, 41, |
| (proliferating) ≧7.0 | 44, 45, 46, 48, 49, 50, 53, 55, 58, 59, 66 |
| −log EC₅₀ [mol/l] | 1, 2, 7, 5, 12, 14, 17, 20, 23, 28, 29, 35, 36, 42, |
| RKO p27 uninduced | 43, 47, 51, 52, 54, 56, 57, 60, 62, 63, 64, 65 |
| (proliferating) <7.0 but | |
| ≧6.0 | |

The value of −log EC₅₀ [mol/l] RKO p27 induced (arrested) was below the minimum determined by the assay specification (≦4.0, ≦5.0, ≦5.5 or ≦6.0).

The induction of apoptosis can be measured by using a Cell death detection ELISA (Roche Biochemicals, Mannheim, Germany). NCI-H460 non-small cell lung cancer cells are seeded into 96 well flat bottom plates at a density of 10000 cells per well in a volume of 50 μl RPMI medium (containing 10% fetal calf serum) per well. The compounds are dissolved as 10 mM solutions in DMSO and subsequently diluted in semi-logarithmic steps. DMSO dilutions are further diluted 1:100 into RPMI medium (containing 10% fetal calf serum) to a final concentration twice as much as the final concentration in the test. 24 hours after seeding the 50 μl each of the compound dilutions in RPMI medium are added into each well of the 96 Well plate. Each compound dilution is tested at least as duplicates. Wells containing untreated control cells are filled with 50 μl RPMI medium containing 1% DMSO. The cells are then incubated with the substances for 24 hours at 37° C. in a humidified atmosphere containing 5% carbon dioxide. As a positive control for the induction of apoptosis, cells are treated with 50 μM Cisplatin (Gry Pharmaceuticals, Kirchzarten, Germany). Medium is then removed and the cells are lysed in 200 μl lysis buffer. After centrifugation as described by the manufacturer, 10 μl of cell lysate is processed as described in the protocol. The degree of apoptosis is calculated as follows: The absorbance at 405 nm obtained with lysates from cells treated with 50 μM cisplatin is set as 100 cpu (cisplatin units), while an absorbance at 405 nm of 0.0 is set as 0.0 cpu. The degree of apoptosis is expressed as cpu in relation to the value of 100 cpu reached with the lysates obtained from cells treated with 50 μM cisplatin.

Experimental perturbation of Eg5 function causes a characteristic malformation of the mitotic spindle, which can be examined by confocal laser scanning microscopy. HeLa cervical cancer cells are grown overnight on glass cover slips (Nunc™ Lab-Tek™ Chamber Slides) in 1800 μl DMEM medium containing 10% fetal calf serum. The test compounds are dissolved as 10 mM solutions in DMSO. Appropriate DMSO dilutions of the test compounds are further diluted 1:20 into DMEM medium containing 10% fetal calf serum to a final concentration ten times as much as the final concentration in the test. 24 hours after seeding, 200 μl of the compound dilutions in DMEM medium are added into each well of the cover slip. As a control, 200 μl DMEM medium containing 5% DMSO are added. 24 hours after incubation with the test compounds, the cells are washed with PBS, and fixed with 3.7% formaldehyde in H₂O for 20 min. at 37° C. Subsequently, cells are washed with PBS and incubated with 0.1% Triton X-100 in a buffer containing 1.471 mM KH₂PO_{4,} 8.504 mM Na₂HPO₄, 137 mM NaCl, 1.325 mM CaCl₂, 2.685 mM KCl, 0.542 mM MgCl₂, pH 7.2 for 15 min. at room temperature. For saturation of non-specific binding, cells are incubated in 2% BSA/10% FCS in PBS (=blocking buffer) for 30 min. at room temperature pior to incubation with anti-alpha tubulin monoclonal antibodies (Sigma, #T5168; 1:1000), followed by Cy3-conjugated rabbit anti-mouse IgG (H+L) antibody (Jackson Immuno Research; 1:1000). All antibody incubations are performed for one hour at 37° C. in blocking buffer, and cells are washed three times in PBS between different incubations. DNA is counterstained with Hoechst 33342 (0.1 μg/ml). Coverslips are mounted in Vectashield (Vector Laboratories, Burlingame, Calif.) and examined with a Leica TCS SP2 confocal laser scanning microscope fitted with appropriate filters (Leica Microsystems, Bensheim, Germany).

Some of the compounds according to this invention may be efficacious against p-glycoprotein mediated multidrug-resistent tumour cell lines (e.g. HCT-15), that can be measured as follows: All cell lines used are cultured at standard conditions in a tissue culture incubator at 37° C., 5% CO₂ and 95% humidity. At day 1, cells are detached with Trypsin/EDTA and pelleted by centrifugation. Cells are resuspended at the appropriate density in culture medium, seeded into 96 well microtiter plates and incubated over night in a tissue culture incubator at 37° C., 5% CO₂ and 95% humidity. Stock solution of all compounds to be tested are dissolved at 10 mM in DMSO and at day 2 added to the microtiter plates in the desired dilutions. The final DMSO concentration in the microtiter plates is kept at 0.5%. Control cells are treated with culture medium including a final concentration of 0.5% DMSO only. The microtiter plates are incubated with the compounds in a tissue culture incubator at 37° C., 5% CO₂ and 95% humidity for further 72 hours. To determine the viability of the cells at day 5, an Alamar Blue solution (Biosource) is added at 1/10 culture volume to the microtiter plates. The cells are incubated in a tissue culture incubator at 37° C., 5% CO₂ and 95% humidity for additional 1-6 hours and the fluorescence is measured at an extinction of 544 nm and an emission of 590 nm. For the calculation of the cell viability the emission value from untreated cells is set as 100% viability and the emission rates of treated cells are set in relation to the values of untreated cells. Viabilities are expressed as % values.

The Graphpad Prism program is used for the calculation of EC₅₀ values out of the obtained dose-response curves.

1. Compounds of formula I 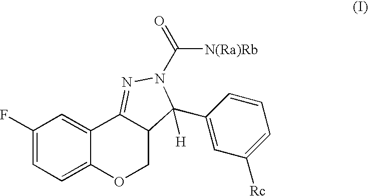
in which
Ra is 1-4C-alkyl, 3-7C-cycloalkyl, 3-7C-cycloalkyl-1-4C-alkyl, HetA, HetA-1-4C-alkyl, completely or partially fluorine-substituted 1-4C-alkyl, or 1-4C-alkyl substituted by Raa,
wherein said 3-7C-cycloalkyl alone or as part of another group may be optionally substituted by one or two substituents independently selected from —N(R2)R3 and R1,
in which
Raa is —N(R2)R3, chlorine, bromine, hydroxyl, or 1-4C-alkoxy,
HetA is tetrahydropyranyl, tetrahydrofuranyl, 1N—(R10)-piperidinyl, or 1N—(R10)-pyrrolidinyl, in which
R10 is hydrogen, 1-4C-alkyl, 3-7C-cycloalkyl, 3-7C-cycloalkyl-1-4C-alkyl, 1-4C-alkylcarbonyl, amidino, or completely or partially fluorine-substituted 1-4C-alkyl,
wherein said HetA alone or as part of another group may be optionally substituted by one or two substituents independently selected from R1,
R1 is fluorine, or completely or partially fluorine-substituted 1-4C-alkyl,
R2 is hydrogen, 1-4C-alkyl, 3-7C-cycloalkyl, 3-7C-cycloalkyl-1-4C-alkyl, 2-4C-alkenyl, 2-4C-alkynyl, 1-4C-alkoxycarbonyl, hydroxy-2-4C-alkyl, 1-4C-alkoxy-2-4C-alkyl, HetA, HetA-1-4C-alkyl, or completely or partially fluorine-substituted 1-4C-alkyl,
R3 is hydrogen, 1-4C-alkyl, 3-7C-cycloalkyl, or 3-7C-cycloalkyl-1-4C-alkyl,
or R2 and R3 together and with inclusion of the nitrogen atom, to which they are bonded, form a ring HetB, in which
HetB is piperidin-1-yl, morpholin-4-yl, thiomorpholin-4-yl, S-oxo-thiomorpholin-4-yl, S,S-dioxo-thiomorpholin-4-yl, pyrrolidin-1-yl, azetidin-1-yl, homopiperidin-1-yl, 4N—(R21)-piperazin-1-yl, 4N—(R21)-homopiperazin-1-yl, pyrrol-1-yl, pyrazol-1-yl, imidazol-1-yl, triazol-1-yl, or tetrazol-1-yl, in which
R21 is hydrogen, 1-4C-alkyl, 3-7C-cycloalkyl, 3-7C-cycloalkyl-1-4C-alkyl, 1-4C-alkylcarbonyl, amidino, or completely or partially fluorine-substituted 1-4C-alkyl,
wherein said HetB may be optionally substituted by one or two substituents independently selected from fluorine and 1-4C-alkyl,
Rb is 1-4C-alkyl, 3-7C-cycloalkyl, or 3-7C-cycloalkyl-1-4C-alkyl,
Rc is hydrogen or hydroxyl,
and the salts, stereoisomers and the salts of the stereoisomers of these compounds.

2. Compounds of formula I according to claim 1, in which
Ra is 1-4C-alkyl, 3-7C-cycloalkyl, 3-7C-cycloalkyl-1-4C-alkyl, HetA, HetA-1-4C-alkyl, completely or partially fluorine-substituted 1-4C-alkyl, or 1-4C-alkyl substituted by Raa,
wherein said 3-7C-cycloalkyl alone or as part of another group may be optionally substituted by one or two substituents independently selected from —N(R2)R3 and R1,
in which
Raa is —N(R2)R3, chlorine, bromine, hydroxyl, or 1-4C-alkoxy,
HetA is tetrahydropyranyl, tetrahydrofuranyl, 1N—(R10)-piperidinyl, or 1N—(R10)-pyrrolidinyl, in which
R10 is hydrogen, 1-4C-alkyl, 3-7C-cycloalkyl, 3-7C-cycloalkyl-1-4C-alkyl, 1-4C-alkylcarbonyl, amidino, or completely or partially fluorine-substituted 1-4C-alkyl,
wherein said HetA alone or as part of another group may be optionally substituted by one or two substituents independently selected from R1,
R1 is fluorine, or completely or partially fluorine-substituted 1-4C-alkyl,
R2 is hydrogen, 1-4C-alkyl, 3-7C-cycloalkyl, 3-7C-cycloalkyl-1-4C-alkyl, hydroxy-2-4C-alkyl, 1-4C-alkoxy-2-4C-alkyl, HetA, HetA-1-4C-alkyl, or completely or partially fluorine-substituted 1-4C-alkyl,
R3 is hydrogen, 1-4C-alkyl, 3-7C-cycloalkyl, or 3-7C-cycloalkyl-1-4C-alkyl,
or R2 and R3 together and with inclusion of the nitrogen atom, to which they are bonded, form a ring HetB, in which
HetB is piperidin-1-yl, morpholin-4-yl, thiomorpholin-4-yl, S-oxo-thiomorpholin-4-yl, S,S-dioxo-thiomorpholin-4-yl, pyrrolidin-1-yl, azetidin-1-yl, homopiperidin-1-yl, 4N—(R21)-piperazin-1-yl, 4N—(R21)-homopiperazin-1-yl, pyrrol-1-yl, pyrazol-1-yl, imidazol-1-yl, triazol-1-yl, or tetrazol-1-yl, in which
R21 is hydrogen, 1-4C-alkyl, 3-7C-cycloalkyl, 3-7C-cycloalkyl-1-4C-alkyl, 1-4C-alkylcarbonyl, amidino, or completely or partially fluorine-substituted 1-4C-alkyl,
wherein said HetB may be optionally substituted by one or two substituents independently selected from fluorine and 1-4C-alkyl,
Rb is 1-4C-alkyl, 3-7C-cycloalkyl, or 3-7C-cycloalkyl-1-4C-alkyl,
Rc is hydrogen or hydroxyl,
and the salts, stereoisomers and the salts of the stereoisomers of these compounds.

3. Compounds of formula I according to claim 2, in which
Ra is methyl, ethyl, propyl, isopropyl, isobutyl, amino-cyclohexyl, HetA, HetA-methyl, 2-(Raa)-ethyl, or 3-(Raa)-propyl,
in which
Raa is —N(R2)R3,
either
HetA is tetrahydropyranyl, 1N—(R10)-piperidinyl, or 1N—(R10)-pyrrolidinyl, in which
R10 is hydrogen, methyl, ethyl, isopropyl, acetyl, 2-fluoroethyl, 2,2-difluoroethyl, or 2,2,2-trifluoroethyl,
or
HetA is 1N—(R10)-fluoropiperidinyl, 1N—(R10)-fluoropyrrolidinyl, 1N—(R10)-(fluoromethyl)pyrrolidinyl, or 1N-(R10)-(fluoromethyl)piperidinyl, such as, for example, (3S,4R)-3-fluoro-1N—(R10)-piperidin-4-yl, (3R,4S)-3-fluoro-1N—(R10)-piperidin-4-yl, (3R,4R)-3-fluoro-1N—(R10)-piperidin-4-yl, (3S,4S)-3-fluoro-1N—(R10)-piperidin-4-yl, (2R,4R)-2-(fluoromethyl)-1N—(R10)-piperidin-4-yl, (2S,4S)-2-(fluoromethyl)-1N—(R10)-piperidin-4-yl, (3R,4R)-4-fluoro-1N—(R10)-pyrrolidin-3-yl, (3S,4S)-4-fluoro-1N—(R10)-pyrrolidin-3-yl, (3S,5R)-5-fluoromethyl-1N—(R10)-pyrrolidin-3-yl or (3S,5S)-5-fluoromethyl-1N—(R10)-pyrrolidin-3-yl, in which
R10 is hydrogen, methyl, ethyl, isopropyl, acetyl, 2-fluoroethyl, 2,2-difluoroethyl, or 2,2,2-trifluoroethyl,
in which
either
R2 is hydrogen, and
R3 is hydrogen,
or
R2 is methyl, and
R3 is hydrogen,
or
R2 is ethyl, and
R3 is hydrogen,
or
R2 is methyl, and
R3 is methyl,
or
R2 is ethyl, and
R3 is methyl,
or
R2 is ethyl, and
R3 is ethyl,
or
R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, 4-methyl-piperazin-1-yl, 4-acetyl-piperazin-1-yl, 4-methyl-piperidin-1-yl, 4-fluoro-piperidin-1-yl, 4,4-difluoro-piperidin-1-yl, (S)-3-fluoro-pyrrolidin-1-yl, (R)-3-fluoro-pyrrolidin-1-yl, 3,3-difluoro-pyrrolidin-1-yl, 3-fluoro-azetidin-1-yl, 3,3-difluoro-azetidin-1-yl, or imidazol-1-yl,
Rb is methyl, ethyl, isopropyl, or cyclopropyl,
Rc is hydrogen or hydroxyl,
and the salts, stereoisomers and the salts of the stereoisomers of these compounds.

4. Compounds of formula I according to claim 1, which are from formula I* 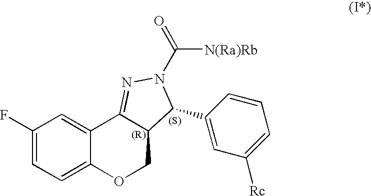
and the salts, stereoisomers and the salts of the stereoisomers of these compounds.

5. Compounds of formula I according to claim 2, which are from formula I** 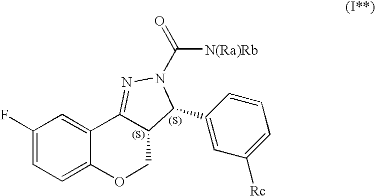
and the salts, stereoisomers and the salts of the stereoisomers of these compounds.

6. Compounds of formula I according to claim 1, in which
Rb is methyl,
and the salts, stereoisomers and the salts of the stereoisomers of these compounds.

7. Compounds of formula I, I* or I** according to claim 1, in which
Ra is methyl, ethyl, propyl, isopropyl, isobutyl, HetA, HetA-methyl, 2-(Raa)-ethyl, or 3-(Raa)-propyl,
in which
Raa is —N(R2)R3,
HetA is 1-methyl-piperidin-4-yl, 1H-piperidin-4-yl, 1-methyl-piperidin-3-yl, 1H-piperidin-3-yl, 1-isopropyl-piperidin-4-yl, or 1-isopropyl-piperidin-3-yl,
in which
either
R2 is hydrogen, and
R3 is hydrogen,
or
R2 is methyl, and
R3 is hydrogen,
or
R2 is ethyl, and
R3 is hydrogen,
or
R2 is methyl, and
R3 is methyl,
or
R2 and R3 together and with inclusion of the nitrogen atom, to which they are bonded, form a ring HetB, in which
HetB is piperidin-1-yl, morpholin-4-yl, pyrrolidin-1-yl, azetidin-1-yl, 4-methyl-piperazin-1-yl, or 4-acetyl-piperazin-1-yl,
Rb is methyl, ethyl, or cyclopropyl,
Rc is hydroxyl or hydrogen,
and the salts, stereoisomers and the salts of the stereoisomers of these compounds.

8. Compounds of formula I according to claim 1, in which
Ra is 1N—(R10)-piperidin-3-yl or 1N—(R10)-piperidin-4-yl, in which
R10 is hydrogen, methyl, ethyl, isopropyl or cyclopropyl,
Rb is methyl, ethyl, isopropyl or cyclopropyl,
Rc is hydrogen or hydroxyl,
and the salts, stereoisomers and the salts of the stereoisomers of these compounds.

9. Compounds of formula I according to claim 1, in which
Ra is 1-4C-alkyl, HetA, HetA-1-4C-alkyl, or 1-4C-alkyl substituted by Raa,
in which
Raa is —N(R2)R3 or chlorine,
HetA is 1N—(R10)-piperidinyl, in which
R10 is hydrogen, 1-4C-alkyl, or 1-4C-alkylcarbonyl,
R2 is hydrogen, 1-4C-alkyl, 3-7C-cycloalkyl, 3-7C-cycloalkyl-1-4C-alkyl, 2-4C-alkenyl, 2-4C-alkynyl, 1-4C-alkoxycarbonyl, hydroxy-2-4C-alkyl, 1-4C-alkoxy-2-4C-alkyl, or completely or partially fluorine-substituted 1-4C-alkyl,
R3 is hydrogen, 1-4C-alkyl,
or R2 and R3 together and with inclusion of the nitrogen atom, to which they are bonded, form a ring HetB, in which
HetB is piperidin-1-yl, azetidin-1-yl, 4N—(R21)-piperazin-1-yl, or pyrrol-1-yl, in which
R21 is 1-4C-alkylcarbonyl,
wherein said HetB may be optionally substituted by 1-4C-alkyl,
Rb is 1-4C-alkyl or 3-7C-cycloalkyl,
Rc is hydrogen or hydroxyl,
and the salts, stereoisomers and the salts of the stereoisomers of these compounds.

10. Compounds of formula I according to claim 1, in which
Ra is HetA or 1-4C-alkyl substituted by Raa,
in which
Raa is —N(R2)R3,
HetA is 1N—(R10)-piperidinyl, in which
R10 is hydrogen or methyl,
R2 1-4C-alkyl or hydroxy-2-4C-alkyl,
R3 is hydrogen, 1-4C-alkyl,
or R2 and R3 together and with inclusion of the nitrogen atom, to which they are bonded, form a ring HetB, in which
HetB is piperidin-1-yl, azetidin-1-yl, or pyrrol-1-yl, in which
Rb is 1-4C-alkyl or 3-7C-cycloalkyl,
Rc is hydrogen or hydroxyl,
and the salts, stereoisomers and the salts of the stereoisomers of these compounds.

11. A compound according to claim 1, which is selected from
(1) (3S,3aR)-8-Fluoro-3-(3-hydroxy-phenyl)-3a,4-dihydro-3H-chromeno[4,3-c]pyrazole-2-carboxylic acid dimethylamide
(2) (3S,3aS)-8-Fluoro-3-(3-hydroxy-phenyl)-3a,4-dihydro-3H-chromeno[4,3-c]pyrazole-2-carboxylic acid dimethylamide
(3) (3S,3aS)-8-Fluoro-3-(3-hydroxy-phenyl)-3a,4-dihydro-3H-chromeno[4,3-c]pyrazole-2-carboxylic acid methyl-(1-methyl-piperidin-4-yl)-amide
(4) (3S,3aS)-8-Fluoro-3-(3-hydroxy-phenyl)-3a,4-dihydro-3H-chromeno[4,3-c]pyrazole-2-carboxylic acid (2-dimethylamino-ethyl)-methyl-amide
(5) (3S,3aS)-8-Fluoro-3-(3-hydroxy-phenyl)-3a,4-dihydro-3H-chromeno[4,3-c]pyrazole-2-carboxylic acid cyclopropyl-(1-methyl-piperidin-4-yl)-amide
(6) (3S,3aS)-8-Fluoro-3-(3-hydroxy-phenyl)-3a,4-dihydro-3H-chromeno[4,3-c]pyrazole-2-carboxylic acid (3-dimethylamino-propyl)-methyl-amide
(7) (3S,3aS)-8-Fluoro-3-phenyl-3a,4-dihydro-3H-chromeno[4,3-c]pyrazole-2-carboxylic acid dimethylamide
(8) (3S,3aR)-8-Fluoro-3-phenyl-3a,4-dihydro-3H-chromeno[4,3-c]pyrazole-2-carboxylic acid dimethylamide
(9) (3S,3aS)-8-Fluoro-3-phenyl-3a,4-dihydro-3H-chromeno[4,3-c]pyrazole-2-carboxylic acid methyl-(1-methyl-piperidin-4-yl)-amide
(10) (3S,3aS)-8-Fluoro-3-phenyl-3a,4-dihydro-3H-chromeno[4,3-c]pyrazole-2-carboxylic acid (2-dimethylamino-ethyl)-ethyl-amide
(11) (3S,3aS)-8-Fluoro-3-phenyl-3a,4-dihydro-3H-chromeno[4,3-c]pyrazole-2-carboxylic acid (3-dimethylamino-propyl)-methyl-amide
(12) (3S,3aS)-8-Fluoro-3-phenyl-3a,4-dihydro-3H-chromeno[4,3-c]pyrazole-2-carboxylic acid isobutyl-methyl-amide
(13) (3S,3aS)-8-Fluoro-3-phenyl-3a,4-dihydro-3H-chromeno[4,3-c]pyrazole-2-carboxylic acid isopropyl-methyl-amide
(14) (3S,3aS)-8-Fluoro-3-phenyl-3a,4-dihydro-3H-chromeno[4,3-c]pyrazole-2-carboxylic acid ethyl-methyl-amide
(15) (3S,3aS)-8-Fluoro-3-phenyl-1,3a,4,9b-tetrahydro-3H-chromeno[4,3-c]pyrazole-2-carboxylic acid cyclopropyl-(1-methyl-piperidin-4-yl)-amide
(16) (3S,3aS)-8-Fluoro-3-phenyl-3a,4-dihydro-3H-chromeno[4,3-c]pyrazole-2-carboxylic acid methyl-piperidin-4-yl-amide
(17) (3S,3aS)-8-Fluoro-3-phenyl-3a,4-dihydro-3H-chromeno[4,3-c]pyrazole-2-carboxylic acid cyclopropyl-piperidin-4-yl-amide
(18) (3S,3aS)-8-Fluoro-3-phenyl-3a,4-dihydro-3H-chromeno[4,3-c]pyrazole-2-carboxylic acid (2-amino-ethyl)-methyl-amide
(19) (3S,3aS)-8-Fluoro-3-phenyl-3a,4-dihydro-3H-chromeno[4,3-c]pyrazole-2-carboxylic acid (2-dimethylamino-ethyl)-methyl-amide
(20) (3S,3aS)-8-Fluoro-3-phenyl-3a,4-dihydro-3H-chromeno[4,3-c]pyrazole-2-carboxylic acid (3-amino-propyl)-methyl-amide
(21) (3S,3aS)-8-Fluoro-3-phenyl-3a,4-dihydro-3H-chromeno[4,3-c]pyrazole-2-carboxylic acid (1-acetyl-piperidin-4-yl)-cyclopropyl-amide
(22) (3S,3aS)-8-Fluoro-3-phenyl-3a,4-dihydro-3H-chromeno[4,3-c]pyrazole-2-carboxylic acid methyl-(3-methylamino-propyl)-amide
(23) (3S,3aS)-8-Fluoro-3-phenyl-3a,4-dihydro-3H-chromeno[4,3-c]pyrazole-2-carboxylic acid methyl-(2-methylamino-ethyl)-amide
(24) (3S,3aS)-8-Fluoro-3-phenyl-3a,4-dihydro-3H-chromeno[4,3-c]pyrazole-2-carboxylic acid methyl-((RS)-1-methyl-piperidin-3-ylmethyl)-amide
(25) (3S,3aS)-8-Fluoro-3-phenyl-3a,4-dihydro-3H-chromeno[4,3-c]pyrazole-2-carboxylic acid (RS)-methyl-piperidin-3-yl-amide
(26) (3S,3aS)-8-Fluoro-3-phenyl-3a,4-dihydro-3H-chromeno[4,3-c]pyrazole-2-carboxylic acid ((RS)-1-isopropyl-piperidin-3-ylmethyl)-methyl-amide
27) (3S,3aS)-8-Fluoro-3-(3-hydroxy-phenyl)-3a,4-dihydro-3H-chromeno[4,3-c]pyrazole-2-carboxylic acid methyl-piperidin-4-yl-amide
(28) (3S,3aS)-8-Fluoro-3-phenyl-3a,4-dihydro-3H-chromeno[4,3-c]pyrazole-2-carboxylic acid methyl-(2-pyrrolidin-1-yl-ethyl)-amide and
(29) (3S,3aS)-8-Fluoro-3-phenyl-3a,4-dihydro-3H-chromeno[4,3-c]pyrazole-2-carboxylic acid [2-(4-acetyl-piperazin-1-yl)-ethyl]-methyl-amide
or a salt, stereoisomer or salt of a stereoisomer thereof.

12. A compound according to claim 1, which is selected from
(1) (3RS,3aSR)-8-Fluoro-3-(3-hydroxy-phenyl)-3a,4-dihydro-3H-chromeno[4,3-c]pyrazole-2-carboxylic acid dimethylamide,
(2) (3RS,3aRS)-8-Fluoro-3-(3-hydroxy-phenyl)-3a,4-dihydro-3H-chromeno[4,3-c]pyrazole-2-carboxylic acid dimethylamide,
(3) (3RS,3aRS)-8-Fluoro-3-(3-hydroxy-phenyl)-3a,4-dihydro-3H-chromeno[4,3-c]pyrazole-2-carboxylic acid methyl-(1-methyl-piperidin-4-yl)-amide,
(4) (3RS,3aRS)-8-Fluoro-3-(3-hydroxy-phenyl)-3a,4-dihydro-3H-chromeno[4,3-c]pyrazole-2-carboxylic acid (2-dimethylamino-ethyl)-methyl-amide,
(5) (3RS,3aRS)-8-Fluoro-3-(3-hydroxy-phenyl)-3a,4-dihydro-3H-chromeno[4,3-c]pyrazole-2-carboxylic acid cyclopropyl-(1-methyl-piperidin-4-yl)-amide,
(6) (3RS,3aRS)-8-Fluoro-3-(3-hydroxy-phenyl)-3a,4-dihydro-3H-chromeno[4,3-c]pyrazole-2-carboxylic acid (3-dimethylamino-propyl)-methyl-amide,
(7) (3RS,3aRS)-8-Fluoro-3-phenyl-3a,4-dihydro-3H-chromeno[4,3-c]pyrazole-2-carboxylic acid dimethylamide,
(8) (3RS,3aSR)-8-Fluoro-3-phenyl-3a,4-dihydro-3H-chromeno[4,3-c]pyrazole-2-carboxylic acid dimethylamide,
(9) (3RS,3aRS)-8-Fluoro-3-phenyl-3a,4-dihydro-3H-chromeno[4,3-c]pyrazole-2-carboxylic acid methyl-(1-methyl-piperidin-4-yl)-amide,
(10) (3RS,3aRS)-8-Fluoro-3-phenyl-3a,4-dihydro-3H-chromeno[4,3-c]pyrazole-2-carboxylic acid (2-dimethylamino-ethyl)-ethyl-amide,
(11) (3RS,3aRS)-8-Fluoro-3-phenyl-3a,4-dihydro-3H-chromeno[4,3-c]pyrazole-2-carboxylic acid (3-dimethylamino-propyl)-methyl-amide,
(12) (3RS,3aRS)-8-Fluoro-3-phenyl-3a,4-dihydro-3H-chromeno[4,3-c]pyrazole-2-carboxylic acid isobutyl-methyl-amide,
(13) (3RS,3aRS)-8-Fluoro-3-phenyl-3a,4-dihydro-3H-chromeno[4,3-c]pyrazole-2-carboxylic acid isopropyl-methyl-amide,
(14) (3RS,3aRS)-8-Fluoro-3-phenyl-3a,4-dihydro-3H-chromeno[4,3-c]pyrazole-2-carboxylic acid ethyl-methyl-amide,
(15) (3RS,3aRS)-8-Fluoro-3-phenyl-1,3a,4,9b-tetrahydro-3H-chromeno[4,3-c]pyrazole-2-carboxylic acid cyclopropyl-(1-methyl-piperidin-4-yl)-amide,
(16) (3RS,3aRS)-8-Fluoro-3-phenyl-3a,4-dihydro-3H-chromeno[4,3-c]pyrazole-2-carboxylic acid methyl-piperidin-4-yl-amide,
(17) (3RS,3aRS)-8-Fluoro-3-phenyl-3a,4-dihydro-3H-chromeno[4,3-c]pyrazole-2-carboxylic acid cyclopropyl-piperidin-4-yl-amide,
(18) (3RS,3aRS)-8-Fluoro-3-phenyl-3a,4-dihydro-3H-chromeno[4,3-c]pyrazole-2-carboxylic acid (2-amino-ethyl)-methyl-amide,
(19) (3RS,3aRS)-8-Fluoro-3-phenyl-3a,4-dihydro-3H-chromeno[4,3-c]pyrazole-2-carboxylic acid (2-dimethylamino-ethyl)-methyl-amide,
(20) (3RS,3aRS)-8-Fluoro-3-phenyl-3a,4-dihydro-3H-chromeno[4,3-c]pyrazole-2-carboxylic acid (3-amino-propyl)-methyl-amide,
(21) (3RS,3aRS)-8-Fluoro-3-phenyl-3a,4-dihydro-3H-chromeno[4,3-c]pyrazole-2-carboxylic acid (1-acetyl-piperidin-4-yl)-cyclopropyl-amide,
(22) (3RS,3aRS)-8-Fluoro-3-phenyl-3a,4-dihydro-3H-chromeno[4,3-c]pyrazole-2-carboxylic acid methyl-(3-methylamino-propyl)-amide,
(23) (3RS,3aRS)-8-Fluoro-3-phenyl-3a,4-dihydro-3H-chromeno[4,3-c]pyrazole-2-carboxylic acid methyl-(2-methylamino-ethyl)-amide,
(24) (3RS,3aRS)-8-Fluoro-3-phenyl-3a,4-dihydro-3H-chromeno[4,3-c]pyrazole-2-carboxylic acid methyl-(1-methyl-piperidin-3-ylmethyl)-amide with (R)- or (S)-methyl-piperidin-3-yl or mixtures thereof,
(25) (3RS,3aRS)-8-Fluoro-3-phenyl-3a,4-dihydro-3H-chromeno[4,3-c]pyrazole-2-carboxylic acid-methyl-piperidin-3-yl-amide with (R)- or (S)-methyl-piperidin-3-yl or mixtures thereof,
(26) (3RS,3aRS)-8-Fluoro-3-phenyl-3a,4-dihydro-3H-chromeno[4,3-c]pyrazole-2-carboxylic acid (-1-isopropyl-piperidin-3-ylmethyl)-methyl-amide with (R)- or (S)-1-isopropyl-piperidin-3-yl or mixtures thereof,
(27) (3RS,3aRS)-8-Fluoro-3-(3-hydroxy-phenyl)-3a,4-dihydro-3H-chromeno[4,3-c]pyrazole-2-carboxylic acid methyl-piperidin-4-yl-amide,
(28) (3RS,3aRS)-8-Fluoro-3-phenyl-3a,4-dihydro-3H-chromeno[4,3-c]pyrazole-2-carboxylic acid methyl-(2-pyrrolidin-1-yl-ethyl)-amide,
(29) ((3RS,3aRS)-8-Fluoro-3-phenyl-3a,4-dihydro-3H-chromeno[4,3-c]pyrazole-2-carboxylic acid [2-(4-acetyl-piperazin-1-yl)-ethyl]-methyl-amide,
or a salt, stereoisomer or salt of a stereoisomer thereof.

13. Process for preparing a compound according to claim 1, comprising at least one of the steps
(i) conversion of a compound of formula IIIa′ or IIIa″ and their enantiomers into compounds of formula I** or I* and their enantiomers, wherein Ra, Rb and Rc have the meanings indicated in formula I 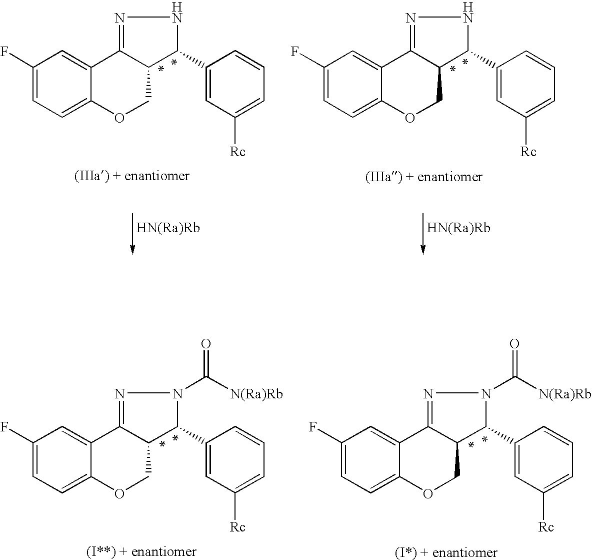
(ii) the conversion according to (i), wherein the compound of formula IIIa′ or IIIa″ and their enantiomers are activated via conversion to the corresponding carbamoylchlorides of formulae IIa′ and IIa″, in which L is chlorine, or via conversion to the corresponding activated carbamates, in which L is 4-nitrophenoxy, and in which Rc has the meanings indicated in formula I, 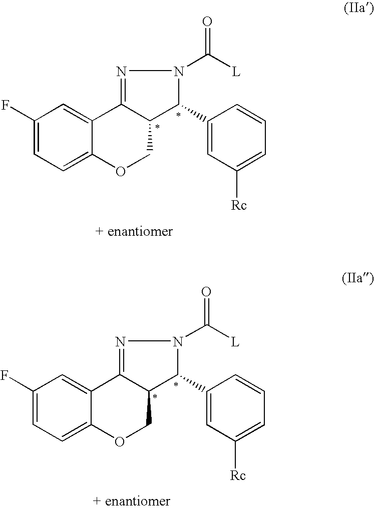
(iii) the conversion according to (i) or (ii), wherein the diastereomers or the enantiomers or both are separated, or
(iv) the conversion according to (i), (ii) or (iii), optionally followed by the removal of at least one temporary protective group selected from (a) a temporary protective group —OPG1 protecting Rc, wherein the removal comprises the conversion from Rc=-OPG1 to Rc=hydroxyl, or (b) a temporary protective group protecting an amino group that is part of the substituent Ra, wherein Ra has the meanings indicated in formula I,
(v) the conversion of a compound according to claim 1 into a corresponding salt of the compound according to claim 1.

14. Compounds according to claim 1 for use in the treatment of diseases.

15. A pharmaceutical composition comprising one or more compounds according to claim 1 together with customary pharmaceutical auxiliaries and/or excipients.

16. A method for treating (hyper)proliferative diseases and/or disorders responsive to induction of apoptosis, such as, for example, benign and/or malignant neoplasia, e.g. cancer, comprising administering to the patient an effective amount of a compound according to claim 1.

17. Pharmaceutical composition for treating (hyper)proliferative diseases and/or disorders responsive to induction of apoptosis, which include benign neoplasia and malignant neoplasia, including cancer, comprising a compound according to claim 1.

18. A method for treating, preventing or ameliorating (hyper)proliferative diseases and/or disorders responsive to induction of apoptosis, such as, for example, benign or malignant neoplasia, e.g. cancer, in a mammal comprising administering a therapeutically effective and tolerable amount of one or more compounds according to claim 1 to said mammal in need thereof.

19. A method for modulating Eg5 kinesin activity comprising administering a therapeutically effective and tolerable amount of one or more compounds according to claim 1 to a mammal in need of said modulation.

20. A combination comprising
a first active ingredient, which is at least one compound according to claim 1, and a second active ingredient, which is at least one anti-cancer agent selected from the group consisting of chemotherapeutic anti-cancer agents and target-specific anti-cancer agents, for separate, sequential, simultaneous, concurrent or chronologically staggered use in therapy, such as e.g. therapy of (hyper)proliferative diseases of benign or malignant behaviour and/or disorders responsive to the induction of apoptosis, such as, for example, benign or malignant neoplasia, e.g. cancer.

21. A method for treating, preventing or ameliorating hyperproliferative diseases and/or disorders responsive to induction of apoptosis, such as, for example, benign or malignant neoplasia, e.g. cancer, in a patient comprising administering separately, simultaneously, concurrently, sequentially or chronologically staggered to said patient in need thereof
an amount of a first active compound, which is a compound according to claim 1, and an amount of at least one second active compound, said second active compound being an anti-cancer agent selected from the group consisting of chemotherapeutic anti-cancer agents and target-specific anti-cancer agents,
wherein the amounts of the first active compound and said second active compound result in a therapeutic effect.

22. The combination according to claim 20, in which said chemotherapeutic anti-cancer agents are selected from (i) alkylating/carbamylating agents including Cyclophosphamid, Ifosfamid, Thiotepa, Melphalan and chloroethylnitrosourea; (ii) platinum derivatives including cis-platin, oxaliplatin, satraplatin and carboplatin; (iii) antimitotic agents/tubulin inhibitors including vinca alkaloids, such as e.g. vincristine, vinblastine or vinorelbine, taxanes, such as e.g. Paclitaxel, Docetaxel and analogs as well as formulations and conjugates thereof, and epothilones, such as e.g. Epothilone B, Azaepothilone or ZK-EPO; (iv) topoisomerase inhibitors including anthracyclines, such as e.g. Doxorubicin, epipodophyllotoxines, such as e.g. Etoposide, and camptothecin and camptothecin analogs, such as e.g. Irinotecan or Topotecan; (v) pyrimidine antagonists including 5-fluorouracil, Capecitabine, Arabinosylcytosine/Cytarabin and Gemcitabine; (vi) purin antagonists including 6-mercaptopurine, 6-thioguanine and fludarabine; and (vii) folic acid antagonists including methotrexate and pemetrexed.

23. The combination according to claim 20 in which said target-specific anti-cancer agents are selected from (i) kinase inhibitors including Imatinib, ZD-1839/Gefitinib, BAY43-9006/Sorafenib, SU11248/Sunitinib and OSI-774/Erlotinib; (ii) proteasome inhibitors including PS-341/Bortezomib; (iii) histone deacetylase inhibitors including SAHA, PXD101, MS275, MGCD0103, Depsipeptide/FK228, NVP-LBH589, NVP-LAQ824, Valproic acid (VPA) and butyrates; (iv) heat shock protein 90 inhibitors including 17-allylaminogeldanamycin (17-AAG); (v) vascular targeting agents (VAT) including combretastatin A4 phosphate and AVE8062/AC7700, and anti-angiogenic drugs including VEGF antibodies, such as e.g. Bevacizumab, and KDR tyrosine kinase inhibitors, such as e.g. PTK787/ZK222584 (Vatalanib); (vi) monoclonal antibodies including Trastuzumab, Rituximab, Alemtuzumab, Tositumab, Cetuximab and Bevacizumab as well as mutants and conjugates of monoclonal antibodies, such as e.g. Gemtuzumab ozogamicin or Ibritumomab tiuxetan, and antibody fragments; (vii) oligonucleotide based therapeutics including G-3139/Oblimersen; (viii) Toll-like receptor/TLR 9 agonists including Promune®, TLR 7 agonists including Imiquimod and Isatoribine and analogues thereof, or TLR 7/8 agonists including Resiquimod as well as immunostimulatory RNA as TLR 7/8 agonists; (ix) protease inhibitors; (x) hormonal therapeutics including anti-estrogens, such as e.g. Tamoxifen or Raloxifen, anti-androgens, such as e.g. Flutamide or Casodex, LHRH analogs, such as e.g. Luprolide, Goserelin or Triptorelin, and aromatase inhibitors; bleomycin; retinoids including all-trans retinoic acid (ATRA); DNA methyltransferase inhibitors including the 2-deoxycytidine derivative Decitabine and 5-azacytidine; alanosine; cytokines including interleukin-2; interferons including interferon α2 and interferon-γ; and death receptor agonists including TRAIL, DR4/5 agonistic antibodies, FasL and TNF-R agonists.

24. The method according to claim 16 in which said cancer is selected from the group consisting of cancer of the breast, bladder, bone, brain, central and peripheral nervous system, colon, endocrine glands, esophagus, endometrium, germ cells, head and neck, kidney, liver, lung, larynx and hypopharynx, mesothelioma, sarcoma, ovary, pancreas, prostate, rectum, renal, small intestine, soft tissue, testis, stomach, skin, ureter, vagina and vulva; inherited cancers, retinomblastoma and Wilms tumor; leukemia, lymphoma, non-Hodgkins disease, chronic and acute myeloid leukemia, acute lymphoblastic leukemia, Hodgkins disease, multiple myeloma and T-cell lymphoma; myelodysplastic syndrome, plasma cell neoplasia, paraneoplastic syndromes, cancers of unknown primary site and AIDS related malignancies.
